(19) United States
(12) Patent Application Publication
(10) Pub. No.: US 2025/0090655 A1
(43) Pub. Date: Mar. 20, 2025

(54) BOOSTING SARS-COV-2 IMMUNITY WITH A LENTIVIRAL-BASED NASAL VACCINE

(71) Applicants: INSTITUT PASTEUR, PARIS CEDEX 15 (FR); THERAVECTYS, PARIS CEDEX 15 (FR)

(72) Inventors: Pierre CHARNEAU, PARIS (FR); Laleh MAJLESSI, PARIS (FR); Maryline BOURGINE, PARIS (FR); Benjamin VESIN, PARIS (FR); Jodie LOPEZ, PARIS (FR); Ingrid FERT, PARIS (FR)

(21) Appl. No.: 18/726,286

(22) PCT Filed: Jan. 17, 2023
(86) PCT No.: PCT/EP2023/051025

§ 371 Date: Jul. 2, 2024

(30) Foreign Application Priority Data
Jan. 17, 2022 (WO) PCT/IB2022/000035

(51) Int. Cl. A61K 39/215; A61K 39/00; A61P 37/04

(52) U.S. Cl. CPC A61K 39/215; A61P 37/04; A61K 2039/5256; A61K 2039/543

(57) ABSTRACT

The invention relates to the field of immunity against coronaviruses. In this respect, the invention provides a lentiviral-based immunogenic agent that is suitable for use in boost or target immunization treatment in a subject, in particular a human subject, who had previously developed an immunity against Severe Acute Respiratory Syndrome coronavirus 2 (SARS-CoV-2) based on: (i) vaccination with the first generation of vaccines against SARS-CoV-2 infection or disease such as a protein, an mRNA, an adenovirus, an inactivated virus or a protein subunit vaccine composition against SARS-CoV-2 infection or disease, in particular a protein- or an mRNA-based vaccine, or (ii) SARS-CoV-2-induced or correlated disease. The invention accordingly concerns a lentiviral-based immunogenic agent that in particular may help overcome the deficiencies of available vaccines against SARS-CoV-2, especially may be efficient in overcoming the waning immune response or insufficient cellular memory response observed after immunization with available first generation of vaccines such as a protein, an mRNA, an adenovirus, an inactivated virus or a protein subunit vaccine, in particular protein or mRNA vaccine, by triggering a mucosal humoral and cellular immune response against coronaviruses, including a long-lasting immune response.

The invention relates to the field of immunity against coronaviruses. In this respect, the invention provides a lentiviral-based immunogenic agent that is suitable for use in boost or target immunization treatment in a subject, in particular a human subject, who had previously developed an immunity against Severe Acute Respiratory Syndrome coronavirus 2 (SARS-CoV-2) based on: (i) vaccination with a first generation of vaccines against SARS-CoV-2 infection or disease such as a protein, an mRNA, an adenovirus, an inactivated virus or a protein subunit vaccine composition against SARS-CoV-2 infection or disease, in particular a protein- or an mRNA-based vaccine, or (ii) SARS-CoV-2-induced or correlated disease. The invention accordingly concerns a lentiviral-based immunogenic agent that in particular may help overcome the deficiencies of available vaccines against SARS-CoV-2, especially may be efficient in overcoming the waning immune response or insufficient cellular memory response observed after immunization with available first generation of vaccines such as a protein, an mRNA, an adenovirus, an inactivated virus or a protein subunit vaccine, in particular protein or mRNA vaccine, by triggering a mucosal humoral and cellular immune response against coronaviruses, including a long-lasting immune response.

Considering: (i) the sustained pandemicity of coronavirus disease 2019 (COVID-19), (ii) weakening protection potential of the first-generation vaccines against SARS-CoV-2, and (iii) ceaseless emergence of new viral Variants of Concerns (VOCs), new effective vaccine platforms can be critical for the future primary or booster vaccines (Global COVID-19 Vaccination—Strategic Vision for 2022, World Health Organization, SAGE meeting October 2021). The inventors recently demonstrated the strong performance of a lentiviral vaccination vector (LV) encoding the full-length sequence of Spike glycoprotein (S) from the ancestral SARS-CoV-2 (LV::S), when used in systemic prime followed by intranasal (i.n.) boost in multiple preclinical models (Ku M W, et al. Cell Host Microbe, 29(2), 236-249 e236, 2021). LV::S ensures complete (cross) protection of the respiratory tract against ancestral SARS-CoV-2 and VOCs (Ku M W, et al. EMBO Mol Med, e14459, 2021). In addition, in new transgenic mice, expressing human Angiotensin Converting Enzyme 2 (hACE2) and displaying unprecedented permissiveness of the brain to SARS-CoV-2 replication, an i.n. boost with LV::S is required for full protection of the central nervous system (Ku M W, et al. EMBO Mol Med, e14459, 2021). LV::S is intended to be used as a primary vaccine or a booster to reinforce and broaden protection against emerging VOCs with immune evasion potential (Juno J A, Wheatley A K. Nat Med, 27(11), 1874-1875, 2021).

The duration of the protection conferred by the first generation COVID-19 vaccines is not yet well established, hardly predictable with serological laboratory tests and variable in diverse individuals and against distinct VOCs. Despite high vaccination rates, the current exacerbation of the world-wide pandemic indicates that repeated booster immunizations will be needed to ensure individual and collective immunity against COVID-19. In this context, the safety and potential adverse effects of multiple additional homologous doses of the first generation COVID-19 vaccines, for instance related to allergic reaction to polyethylene glycol (PEG) contained in mRNA vaccines, have to be taken into account (Castells M C, Phillips E J. N Engl J Med, 384(7), 643-649, 2021). Importantly, unmatched vaccine delivery methods, i.e., heterologous prime-boost format, has been proven to be a more successful strategy than homologous prime-boost approach in numerous preclinical models of various infectious diseases (He Q, et al. Emerg Microbes Infect, 10(1), 629-637, 2021; Lu S. Curr Opin Immunol, 21(3), 346-351, 2009; Nordstrom P, et al. Lancet Reg Health Eur, 100249, 2021). Therefore, new efficient vaccination platforms are of particular interest to develop heterologous boosters against COVID-19. The LV::S vaccine candidate has a serious potential for prophylactic use against COVID-19, mainly based on its strong capacity to induce, not only strong neutralizing humoral responses, but also robust protective T-cell responses which are not impacted by the escape mutations accumulated in the SARS-CoV-2 VOCs (Ku M W, et al. EMBO Mol Med, e14459, 2021).

Besides, it has been observed that with the decreasing prophylactic potential of the immunity initially induced by the first-generation vaccines, especially against new VOCs, administration of additional vaccine doses becomes essential (Global COVID-19 Vaccination—Strategic Vision for 2022, World Health Organization, SAGE meeting October 2021). As an alternative to additional doses of the same vaccines, combining vaccine platforms in a heterologous prime-boost regimen may hold promise for gaining protective efficacy (Barros-Martins J, et al. Nat Med, 27(9), 1525-1529, 2021). Compared to homologous vaccine dose administration, heterologous prime-boost strategies may reinforce better the specific adaptive immune responses and long-term protection, without triggering/reinforcing vector-specific immunity or the risk of aggravation of possible reactogenicity to the vaccines themselves or excipients. Furthermore, the sequence of the Spike antigen has to be adapted according to the dynamics of SARS-CoV-2 VOC emergence in order to induce the greatest neutralization breadth. In addition, whereas protection against symptomatic SARS-CoV-2 infection is mainly related to sero-neutralizing activity, protection against severe COVID-19 involves CD8⁺ T-cell immunity. These cells with their ability to cytolyze virus-infected cells, especially controls the virus replication and result in resolution of SARS-CoV-2 infection (Sette A, Crotty S. Cell, 184(4), 861-880, 2021). Therefore, an appropriate B- and T-cell vaccine platform, including an adapted Spike sequence, is of utmost interest at the current step of the pandemic.

The inventors have reasoned that LV::S could be remarkably suitable to be used as a heterologous i.n. booster vaccine, to reinforce and broaden protection against the SARS-CoV-2 in particular against its known and emerging VOCs (including but not limited to Alpha, Beta, Gamma, Delta and Omicron variants of SARS-CoV-2), while collective immunity in early vaccinated nations is waning only a few months after completion of the initial immunization, and while new waves of infections are on the rise (Juno J A, Wheatley A K. Nat Med, 27(11), 1874-1875, 2021).

LVs for use in the present invention are in particular non-integrating, non-replicative, non-cytopathic and negligibly inflammatory (Hu B, Tai A, Wang P. Immunol Rev, 239(1), 45-61, 2011; Ku M W, Charneau P, Majlessi L. Expert Rev Vaccines, 1-16, 2021). These vectors are pseudotyped with the heterologous glycoprotein from Vesicular Stomatitis Virus (VSV-G) which confers them a broad tropism for diverse cell types, notably including dendritic cells. The latter are mainly non-dividing cells and thus barely permissive to gene transfer. Hence, LVs possess the central property to efficiently transfer genes to the nuclei of non-dividing cells, which therefore renders possible efficient transduction of dendritic cells. The resulting endogenous antigen expression in these cells with unique ability to activate naïve T cells (Guermonprez P, et al. J. Int Rev Cell Mol Biol, 349, 1-54, 2019) correlates with outstanding ability of LV at inducing high-quality effector and memory T cells (Ku M W, et al. Commun Biol, 4(1), 713, 2021). Importantly, VSV-G pseudo-typing also avoids LVs to be targets of preexisting vector-specific immunity in humans which is key in vaccine development (Hu B, Tai A, Wang P. Immunol Rev, 239(1), 45-61, 2011; Ku M W, Charneau P, Majlessi L. Expert Rev Vaccines, 1-16, 2021). The safety of LV has been established in humans in a phase I/IIa Human Immunodeficiency Virus-1 therapeutic vaccine trial (EU Clinical Trials Register, Clinical Trials for 2011-006260-52). Because of their non-cytopathic and non-inflammatory properties (Cousin C, et al. Cell Rep, 26(5), 1242-1257 e1247, 2019; Lopez J, et al. An optimized lentiviral vector induces CD4+ T-cell immunity and predicts a booster vaccine against tuberculosis. in revision), LVs are well suitable for mucosal vaccination. The i.n. administration route presents well-recognized advantages of triggering mucosal IgA responses, as well as resident memory B and T lymphocytes in the respiratory tract (Lund F E, Randall T D. Science, 373(6553), 397-399, 2021). This route has also been shown to be the most effective at reducing SARS-CoV-2 transmission in both hamster and macaque preclinical models (van Doremalen N, et al. Sci Transl Med, 13(607), 2021). Induction of mucosal immunity by i.n. immunization allows SARS-CoV-2 neutralization, directly at the gateway to the host organism, before it gains access to major infectable anatomical sites (Ku M W, et al. Cell Host Microbe, 29(2), 236-249 e236, 2021).

In the present invention, the inventors generated an LV encoding the down-selected S_{CoV-2} of the Beta variant, stabilized by K⁹⁸⁶P and V⁹⁸⁷P substitutions in the S2 domain of S_{Cov-2} (LV::S_{Beta-2P}). In mice, primed and boosted intramuscularly (i.m.) with mRNA-1273 (Moderna) vaccine, and in which the (cross) sero-neutralization potential was progressively turning down, the inventors compared the systemic and mucosal immune responses and the protective potential of an i.n. LV::S_{Beta-2P} heterologous boost vs an i.m. mRNA-1273 (Moderna) (Jackson L A, et al. Preliminary Report. N Engl J Med, 383(20), 1920-1931, 2020; Wang F, et al. Med Sci Monit, 26, e924700, 2020) homologous boost. The inventors observed multiple advantages with respect to the former regimen with an improved antigen design, new vaccine delivery LV platform and the alternative i.n. administration route.

According to a first aspect, the invention hence relates to a pseudotyped lentiviral vector particle encoding a Spike (S) protein of a Severe Acute Respiratory Syndrome coronavirus 2 (SARS-CoV-2) or a derivative thereof for use as a heterologous boost or target immunization agent in a vaccine regimen for administration to the upper respiratory tract of a subject, in particular a human subject, who received a prime immunization with a vaccine composition against SARS-CoV-2 infection or disease selected from the group consisting of a protein, an mRNA, an adenovirus, an inactivated virus and a protein subunit vaccine composition against SARS-CoV-2 infection or disease, in particular a protein or an mRNA vaccine composition against SARS-CoV-2 infection or disease. Non-limited examples of protein subunit vaccine compositions against SARS-CoV-2 infection or disease according to the invention may include vaccines based on adjuvanted recombinant Spike protein or vaccines based on recombinant Spike protein packaged in nanoparticles.

Spike (S) protein of SARS-CoV-2 virus is well identified in the art as an envelop-anchored glycoprotein (Walls et al, 2020, Structure, Function, and Antigenicity of the SARS-CoV-2 Spike Glycoprotein. Cell 181:281-292 e286). More precisely, the SARS-CoV-2 S (S_{CoV-2}) is a (180 kDa)₃ homotrimeric class I viral fusion protein, which engages the carboxypeptidase Angiotensin-Converting Enzyme 2 (ACE2), expressed on host cells. The monomer of S_{CoV-2} protein possesses an ecto-domain, a transmembrane anchor domain, and a short internal tail. S_{CoV-2} is activated by a two-step sequential proteolytic cleavage to initiate fusion with the host cell membrane. Subsequent to S_{CoV-2}-ACE2 interaction, which leads to a conformational reorganization, the extracellular domain of S_{CoV-2} is first cleaved at the highly specific furin 682^{RRAR}685 (SEQ ID NO: 21) site (Guo et al., 2020, The origin, transmission and clinical therapies on coronavirus disease 2019 (COVID-19) outbreak—an update on the status. Mil Med Res 7, 11; Walls et al., 2020), a key factor determining the pathological features of the virus, linked to the ubiquitous furin expression (Wang et al., 2020A Unique Protease Cleavage Site Predicted in the Spike Protein of the Novel Pneumonia Coronavirus (2019-nCoV) Potentially Related to Viral Transmissibility. Virol Sin 2020 June; 35(3):337-339. doi: 10.1007/s12250-020-00212-7. Epub 2020 Mar. 20). The resulted subunits are constituted of: (i) S1, which harbors the ACE2 Receptor Binding Domain (RBD), with the atomic contacts restricted to the ACE2 protease domain and also harbors main B-cell epitopes, targeted of neutralizing antibodies (NAbs) (Walls et al., 2020), and (ii) S2, which bears the membrane-fusion elements. Like for S_{CoV-1}, the shedding of S1 renders accessible on S2 the second proteolytic cleavage site 797^{R}, namely S2′ (Belouzard et al., 2009, Activation of the SARS coronavirus spike protein via sequential proteolytic cleavage at two distinct sites. Proc Natl Acad Sci USA 106:5871-5876). According to the cell or tissue types, one or several host proteases, including furin, trypsin, cathepsins or TransMembrane Protease Serine Protease (TMPRSS)-2 or -4, can be involved in this second cleavage step (Coutard et al., 2020, The spike glycoprotein of the new coronavirus 2019-nCoV contains a furin-like cleavage site absent in CoV of the same clade. Antiviral Res 176:104742). The consequent “fusogenic” conformational changes of S result in a highly stable post-fusion form of S_{CoV-2} that initiates the fusion reaction with the host cell membrane (Sternberg and Naujokat, 2020 Structural features of coronavirus SARS-CoV-2 spike protein: Targets for vaccination. Life Sci 257, 118056) and lead to the exposure of a Fusion Peptide (FP), adjacent to S2′. Insertion of FP to the host cell/vesicle membrane primes the fusion reaction, whereby the viral RNA release into the host cytosol (Lai et al., 2017 The SARS-CoV Fusion Peptide Forms an Extended Bipartite Fusion Platform that Perturbs Membrane Order in a Calcium-Dependent Manner. J Mol Biol 429:3875-3892). The facts that the S_{CoV-2}-ACE2 interaction is the only mechanism, thus far identified for the host cell infection by SARS-CoV-2, and that the RBD contains numerous conformational B-cell epitopes (Walls et al., 2020), designate this viral envelop glycoprotein as the main target for neutralization antibodies (NAbs).

The S protein for expression by the lentiviral particles of the invention may originate from a SARS-CoV-2 strain and accordingly maybe characterized by an amino acid sequence that is the native sequence of the viral protein. In a particular embodiment the invention is performed using the S protein of known SARS-CoV-2 strains such as the S protein of the Ancestral strain (wherein the amino acid sequence is SEQ ID NO: 1), or of variant strains discovered later such as the Alpha, Beta, Gamma, Delta or Omicron strain (all regarded as variant strains with respect to one another).

The invention may alternatively be performed with a derivative of the S protein, i.e., a derivative of a native S protein obtained by mutation in the amino acid sequence of the S protein, as will be disclosed herein. In order to express LV::S recombinant particles, the nucleic acid encoding the S protein may have the sequence of the gene present in the viral strain of origin or may be a codon-optimized acid nucleic suitable for expression in mammalian cells, in particular in human cells. In order to express LV recombinant particles expressing a derivative of the S protein, the nucleic acid encoding the derivative of the S protein may have the sequence deduced from the sequence of the gene of the S protein present in a viral strain and may be a codon-optimized acid nucleic suitable for expression in mammalian cells.

In a particular embodiment, the recombinant lentiviral particles (LV) used in the invention are HIV-1-based lentiviral particles. Accordingly, where the expressions “lentiviral particle” of “LV” are used herein it is in particular directed to the HIV-1 based lentiviral particles especially LV particles pseudotyped with VSV-G protein, in particular LV as illustrated in the examples.

The expressions “boost” or “boost immunization” or “boost administration” or “target immunization” refer according to the invention to an administration of the immunogenic agent that comes after a first administration of a heterologous immunization agent, in particular a heterologous vaccine, or after a second or later administration of such heterologous immunization agent or vaccine. Otherwise stated the immunization agent used according to the invention is administered to a subject who previously received a prime administration, or a prime and further one or multiple administration doses, of a heterologous immunization agent or vaccine against the same SARS-CoV-2 or against a variant strain thereof. The boost or target immunization is achieved through administration to the upper respiratory tract, in particular as an intranasal administration, that accordingly distinguish over administration route of a first generation of vaccines against SARS-CoV-2 infection or disease such as a protein, an mRNA, an adenovirus, an inactivated virus or a protein subunit vaccine composition against SARS-CoV-2 infection or disease, in particular protein or mRNA vaccines that most often make use of systemic, including intramuscular, intradermal or subcutaneous administration route. The boost or target immunization is intended to enhance, improve or lengthen the immune response previously raised and possibly to broaden such response to elicit cross-neutralization against multiple SARS-CoV-2 viruses. Improvement of the response may arise from the capability of the immunization agent used in the invention to elicit a mucosal response and to accordingly protect, not only the systemic sites, but also the upper and lower respiratory tracts and the central nervous system that may not have been successfully targeted or protected with heterologous vaccines against SARS-CoV-2 infection or disease such as a protein, an mRNA, an adenovirus, an inactivated virus or a protein subunit vaccine composition against SARS-CoV-2 infection or disease, in particular protein or mRNA vaccines injected via systemic routes. In a particular embodiment the boost administration is intended to raise cross-neutralizing immune response in the subject against emerging stains of the virus. In a particular embodiment, the boost or target immunization may be administered to a subject who received a heterologous immunization agent as disclosed herein and who had and recovered from infection by SARS-CoV-2 or disease related to such infection such as COVID-19. Additional features relating to the use of the immunization agent and to the treatment course of the subject will be disclosed in the following description.

Administration “to the upper respiratory tract” includes any type of administration that results in delivery to the mucosa lining of the upper respiratory tract and includes in particular nasal administration. Administration to the upper respiratory tract includes without limitation, aerosol inhalation, nasal instillation, nasal insufflation, and all combinations thereof. In some embodiments the administration is by aerosol inhalation. In some embodiments the administration is by nasal instillation. In some embodiments the administration is by nasal insufflation.

According to a particular embodiment, the pseudotyped lentiviral vector particle encoding a SARS-CoV-2 S protein or a derivative thereof is for administration as intranasal mucosal boost or target immunization in a subject who received a prime administration with a vaccine composition against SARS-CoV-2 infection or disease selected from the group consisting of a protein, an mRNA, an adenovirus, an inactivated virus and a protein subunit vaccine composition against SARS-CoV-2 infection or disease, in particular a protein or an mRNA vaccine composition against SARS-CoV-2 infection or disease.

According to a particular embodiment, the pseudotyped lentiviral vector particle encoding a S protein of a SARS-CoV-2 or a derivative thereof for use in accordance with the embodiments disclosed herein is further characterized by the following features:
the S protein is from a SARS-CoV-2 virus which is pathogenic for a human host, in particular (i) a S protein from a SARS-CoV-2 virus selected from the group consisting of SARS-CoV-2 Ancestral strain, D614G strain, Alpha strain, Beta strain, Gamma strain, Delta strain and Omicron strain, preferably from Beta strain or Omicron strain, more preferably from Beta strain, or (ii) a S protein from a variant of said Ancestral, D614G strain, Alpha, Beta, Gamma, Delta or Omicron strains wherein such variants encode a S protein with an amino acid sequence at least 90% identical to SEQ ID NO: 1 or,
the S protein is a derivative of the native S protein of one of the Ancestral, D614G, Alpha, Beta, Gamma, Delta or Omicron strains by mutation of 1 to 12, especially 1 to 6 amino acid residues, in particular by substitution and/or deletion of 1 to 12, especially 1 to 6 amino acid residues, in particular (a) is a stabilized form of the S protein characterized by substitution of two consecutive amino acid residues for Proline residues in the S2 domain of the S protein at positions provided as residues 986 and 987 by reference to SEQ ID NO: 1 in the amino acid sequence of the S protein or (b) is a prefusion form of the S protein by deletion of the furin site located from residue 675 to residue 685 by reference to SEQ ID NO: 1 in the amino acid sequence of the S protein or (c) is a stabilized prefusion form of the S protein by deletion of the furin site and substitution of two consecutive amino acid residues for Proline residues in the S2 domain at positions 986 and 987 by reference to SEQ ID NO: 1.

In a particular embodiment, the S protein of the Ancestral strain of SARS-CoV-2 has an amino acid sequence of SEQ ID NO: 1 and the native sequence of the polynucleotide encoding the S protein of the Ancestral strain of SARS-CoV-2 is defined in SEQ ID NO: 2.

In another particular embodiment, the native sequence of the polynucleotide encoding the S protein of the D614G strain of SARS-CoV-2 comprising a mutation of the aspartic acid residue to the glycine residue at position 614 of the amino acid sequence of SEQ ID NO: 1 (D614G), a mutation of the lysine residue to the proline residue at position 986 of the amino acid sequence of SEQ ID NO: 1 (K986P) and a mutation of the valine residue to the proline residue at position 987 of the amino acid sequence of SEQ ID NO: 1 (V987P), i.e. mutation 2P, is defined in SEQ ID NO: 3 (S_{D614G-2P}). The S protein of the D614G strain of SARS-CoV-2 comprising said mutation 2P (S_{D614G-2P}) has an amino acid sequence of SEQ ID NO: 4.

In another particular embodiment, the native sequence of the polynucleotide encoding the S protein of the Alpha strain of SARS-CoV-2 comprising a mutation of the lysine residue to the proline residue at position 986 of the amino acid sequence of SEQ ID NO: 1 (K986P) and a mutation of the valine residue to the proline residue at position 987 of the amino acid sequence of SEQ ID NO: 1 (V987P), i.e. mutation 2P, is defined in SEQ ID NO: 5 (S_{Alpha-2P}). The S protein of the Alpha strain of SARS-CoV-2 comprising said mutation 2P (S_{Alpha-2P}) has an amino acid sequence of SEQ ID NO: 6.

In another particular embodiment, the native sequence of the polynucleotide encoding the S protein of the Beta strain of SARS-CoV-2 is defined in SEQ ID NO: 7 (S_{Beta}). The S protein of the Beta strain of SARS-CoV-2 (S_{Beta}) has an amino acid sequence of SEQ ID NO: 8.

In another particular embodiment, the native sequence of the polynucleotide encoding the S protein of the Beta strain of SARS-CoV-2 comprising a mutation of the lysine residue to the proline residue at position 986 of the amino acid sequence of SEQ ID NO: 1 (K986P) and a mutation of the valine residue to the proline residue at position 987 of the amino acid sequence of SEQ ID NO: 1 (V987P), i.e. mutation 2P, is defined in SEQ ID NO: 9 (S_{Beta-2P}). The S protein of the Beta strain of SARS-CoV-2 comprising said mutation 2P (S_{Beta-2P}) has an amino acid sequence of SEQ ID NO: 10.

In another particular embodiment, the native sequence of the polynucleotide encoding the S protein of the Gamma strain of SARS-CoV-2 comprising a mutation of the lysine residue to the proline residue at position 986 of the amino acid sequence of SEQ ID NO: 1 (K986P) and a mutation of the valine residue to the proline residue at position 987 of the amino acid sequence of SEQ ID NO: 1 (V987P), i.e. mutation 2P, is defined in SEQ ID NO: 11 (S_{Gamma-2P}). The S protein of the Gamma strain of SARS-CoV-2 comprising said mutation 2P (S_{Gamma-2P}) has an amino acid sequence of SEQ ID NO: 12.

In another particular embodiment, the native sequence of the polynucleotide encoding the S protein of the Delta strain of SARS-CoV-2 comprising a mutation of the lysine residue to the proline residue at position 986 of the amino acid sequence of SEQ ID NO: 1 (K986P) and a mutation of the valine residue to the proline residue at position 987 of the amino acid sequence of SEQ ID NO: 1 (V987P), i.e. mutation 2P, is defined in SEQ ID NO: 13 (S_{Delta-2P}). The S protein of the Delta strain of SARS-CoV-2 comprising said mutation 2P (S_{Delta-2P}) has an amino acid sequence of SEQ ID NO: 14.

In another particular embodiment, the native sequence of the polynucleotide encoding the S protein of the Omicron strain of SARS-CoV-2 is defined in SEQ ID NO: 15 (S_{Omicron}). The S protein of the Omicron strain of SARS-CoV-2 (S_{Omicron}) has an amino acid sequence of SEQ ID NO: 16.

In another particular embodiment, the native sequence of the polynucleotide encoding the S protein of the Omicron strain of SARS-CoV-2 comprising a mutation of the lysine residue to the proline residue at position 986 of the amino acid sequence of SEQ ID NO: 1 (K986P) and a mutation of the valine residue to the proline residue at position 987 of the amino acid sequence of SEQ ID NO: 1 (V987P), i.e. mutation 2P, is defined in SEQ ID NO: 17 (S_{Omicron-2P}). The S protein of the Omicron strain of SARS-CoV-2 comprising said mutation 2P (S_{Omicron-2P}) has an amino acid sequence of SEQ ID NO: 18.

In another particular embodiment, the native sequence of the polynucleotide encoding the S protein of the Omicron BA.1 strain of SARS-CoV-2 is defined in SEQ ID NO: 23 (S_{Omicron-BA.1}). The S protein of the Omicron strain of SARS-CoV-2 (S_{Omicron-BA.1}) has an amino acid sequence of SEQ ID NO: 24.

In another particular embodiment, the native sequence of the polynucleotide encoding the S protein of the Omicron BA.4 or BA.5 strain of SARS-CoV-2 is defined in SEQ ID NO: 25 (S_{Omicron-BA.4/5}). The S protein of the Omicron BA.4 or BA.5 strain of SARS-CoV-2 (S_{Omicron-BA.4/5}) has an amino acid sequence of SEQ ID NO: 26.

In another particular embodiment, the native sequence of the polynucleotide encoding the S protein of the Ancestral strain of SARS-CoV-2 comprising a mutation of the lysine residue to the proline residue at position 986 of the amino acid sequence of SEQ ID NO: 1 (K986P) and a mutation of the valine residue to the proline residue at position 987 of the amino acid sequence of SEQ ID NO: 1 (V987P), i.e. mutation 2P, is defined in SEQ ID NO: 19 (S_{2P}). The S protein of the Ancestral strain of SARS-CoV-2 comprising said mutation 2P (S_{2P}) has an amino acid sequence of SEQ ID NO: 20.

In a particular embodiment, the pseudotyped lentiviral vector particle encodes the S protein of the Beta strain of SARS-CoV-2 comprising the mutation 2P (S_{Beta-2P}) that is encoded by the vector pFlap-ieCMV-S-B351-2P-WPREm that has been deposited at the COLLECTION NATIONALE DE CULTURES DE MICROORGANISMES (CNCM) located at Institut Pasteur, 25-28 rue du Docteur Roux, 75724 Paris Cedex 15 FRANCE, on Jul. 6, 2021 under N° CNCM I-5710.

Also provided is the vector pFlap-ieCMV-S-B351-2P-WPREm (CNCM I-5710). The nucleotide sequence of pFlap-ieCMV-S-B351-2P-WPREm is defined in SEQ ID NO: 22.

Also provided is a host cell comprising the vector pFlap-ieCMV-S-B351-2P-WPREm (CNCM I-5710 or SEQ ID NO: 22).

Also provided is a pseudotyped lentiviral vector particle encoding the S protein of the Beta strain of SARS-CoV-2 comprising the mutation 2P (S_{Beta-2P}), wherein the pseudotyped lentiviral vector particle is made by a method comprising co-transfection of a host cell with the vector pFlap-ieCMV-S-B351-2P-WPREm (CNCM I-5710 or SEQ ID NO: 22).

In a particular embodiment, the pseudotyped lentiviral vector particle encoding a S protein of a SARS-CoV-2 or a derivative thereof for use in accordance with the embodiments disclosed herein, is further characterized by the following features: the amino acid sequence of the S protein is SEQ ID NO: 1 or is a derivative thereof having an amino acid sequence at least 90% identical to SEQ ID NO: 1, and the derivative of the S protein of SARS-CoV-2 comprises at least five amino acid mutations including (i) a mutation of the lysine residue to the asparagine residue at position 417 of the amino acid sequence of SEQ ID NO: 1 (K417N), (ii) a mutation of the glutamic acid residue to the lysine residue at position 484 of the amino acid sequence of SEQ ID NO: 1 (E484K) or a mutation of the glutamic acid residue to the alanine residue at position 484 of the amino acid sequence of SEQ ID NO: 1 (E484A), (iii) a mutation of the asparagine residue to the tyrosine residue at position 501 of the amino acid sequence of SEQ ID NO: 1 (N501Y), (iv) a mutation of the lysine residue to the proline residue at position 986 of the amino acid sequence of SEQ ID NO: 1 (K986P) and (v) a mutation of the valine residue to the proline residue at position 987 of the amino acid sequence of SEQ ID NO: 1 (V987P).

In a particular embodiment, the pseudotyped lentiviral vector particle encoding a SS protein of a SARS-CoV-2 or a derivative thereof for use according to the invention, is such that the S protein of SARS-CoV-2 further comprises amino acid mutations selected from the group consisting of (vi) a mutation of the glycine residue to the serine residue at position 446 of the amino acid sequence of SEQ ID NO: 1 (G446S), (vii) a mutation of the threonine residue to the lysine residue at position 478 of the amino acid sequence of SEQ ID NO: 1 (T478K), (viii) a mutation of the glutamine residue to the arginine residue at position 493 of the amino acid sequence of SEQ ID NO: 1 (Q493R) and (ix) a mutation of the glutamine residue to the arginine residue at position 498 of the amino acid sequence of SEQ ID NO: 1 (Q498R).

In a particular embodiment, the pseudotyped lentiviral vector particle encoding a S protein of a SARS-CoV-2 or a derivative thereof for use according to the invention is such that the encoded mutated S protein of SARS-CoV-2 has the amino acid sequence of SEQ ID NO: 10 or SEQ ID NO: 18, preferably of SEQ ID NO: 10.

In another particular embodiment, the pseudotyped lentiviral vector particle encoding a S protein of a SARS-CoV-2 or a derivative thereof for use according to the invention is such that the encoded mutated S protein of SARS-CoV-2 has the amino acid sequence of SEQ ID NO: 24 or SEQ ID NO: 26.

In a particular embodiment, the pseudotyped lentiviral vector particle encoding a S protein of a SARS-CoV-2 or a derivative thereof for use according to the invention is pseudotyped with a vesicular stomatitis virus glycoprotein G (VSV-G) protein.

In particular the VSV-G protein is advantageously provided by a VS virus of the Indiana strain or the New-Jersey strain.

In a particular embodiment, the pseudotyped lentiviral vector particle encoding a S protein of a SARS-CoV-2 or a derivative thereof for use according to the invention is such that the pseudotyped lentiviral vector particle is non-integrative, non-cytopathic and non-replicative.

In accordance with the herein disclosed use of the immunogenic agent according to the invention, in some embodiments, the immunogenic agent or composition comprising the agent is for use in a method of prevention of infection of a human subject by SARS-CoV-2. In some embodiments, the immunogenic agent or composition is for use in a method of protection against SARS-CoV-2 replication in a human subject at risk of being exposed to SARS-CoV-2 or infected by SARS-CoV-2. In some embodiments, the immunogenic composition is for use in a method of preventing development of symptoms or development of a disease associated with infection by SARS-CoV-2, such as COVID-19 in a human subject at risk of being exposed to SARS-CoV-2 or infected by SARS-CoV-2. In some embodiments, the immunogenic composition is for use in a method of preventing the onset of neurological outcome associated with infection by SARS-CoV-2 in a human subject at risk of being exposed to SARS-CoV-2 or infected by SARS-CoV-2. In some embodiments, the immunogenic composition is for use in a method of protecting the Central Nervous System (CNS) of a human subject at risk of being exposed to SARS-CoV-2 or infected by SARS-CoV-2. In some embodiments the vaccine provides protection against the infection by SARS-CoV-2, especially sterilizing protection.

In any of these applications for use in a method disclosed, the immunogenic agent or composition is to be administered to the subject as a prophylactic agent in a boost or target administration step in an effective amount for administration to the upper respiratory tract in order to elicit an immune response against SARS-CoV-2.

In some embodiment the immunogenic composition is for use in a method of protection of a human subject against SARS-CoV-2 infection or against development of the symptoms or the COVID-19 disease associated with SARS-CoV-2 infection, wherein the subject is at risk of developing lung and/or CNS pathology. In particular the human subject is in need of immune protection of CNS from SARS-CoV-2 replication because he/she is affected with comorbid conditions, in particular comorbid conditions affecting the CNS.

In a particular embodiment, the pseudotyped lentiviral vector particle encoding a S protein of a SARS-CoV-2 or a derivative thereof for use according to any one of the embodiments disclosed herein is administered in a subject selected from the group consisting of (a) a subject that has previously received a vaccine composition against SARS-CoV-2 infection or disease selected from the group consisting of a protein, an mRNA, an adenovirus, an inactivated virus and a protein subunit vaccine composition against SARS-CoV-2 infection or disease, in particular a protein- or an mRNA-based vaccine against SARS-CoV-2 infection or disease as a systemic prime and/or boost administration(s) such as intramuscular, intradermal or sub-cutaneous administration(s), in particular an intramuscular prime and/or boost administration(s), (b) a subject that has received a systemic prime administration such as intramuscular, intradermal or sub-cutaneous administration(s), in particular an intramuscular prime administration, of a vaccine composition against SARS-CoV-2 infection or disease selected from the group consisting of a protein, an mRNA, an adenovirus, an inactivated virus and a protein subunit vaccine composition against SARS-CoV-2 infection or disease, in particular a protein- or an mRNA-based vaccine against SARS-CoV-2 infection or disease and has then recovered from coronavirus disease such as coronavirus disease 2019 (COVID-19), (c) a subject that has first recovered from coronavirus disease such as COVID-19 and has then received a systemic prime administration such as intramuscular, intradermal or sub-cutaneous administration(s), in particular an intramuscular administration, of a protein- or an mRNA-based vaccine against SARS-CoV-2 infection or disease, and (d) a subject that has received more than two, in particular more than three, systemic administrations such as intramuscular, intradermal or sub-cutaneous administration(s), in particular an intramuscular administration, of a protein- or an mRNA-based vaccine against SARS-CoV-2 infection or disease.

In a particular embodiment, the pseudotyped lentiviral vector particle encoding a S protein of a SARS-CoV-2 or a derivative thereof is for use in a prime/boost or a target immunization regimen for elicitation of a long-lasting protective mucosal humoral immune response and/or a long-lasting mucosal cellular immune response against SARS-CoV-2 infection or disease, wherein said response protects the respiratory system and/or the CNS of the subject.

In a particular embodiment, the pseudotyped lentiviral vector particle encoding a S protein of a SARS-CoV-2 or a derivative thereof is for use in an immunization regimen wherein the pseudotyped lentiviral vector particle elicits a CD8⁺ T-cell response against SARS-CoV-2.

In a particular embodiment, the pseudotyped lentiviral vector particle encoding a S protein of a SARS-CoV-2 or a derivative thereof is for use in an immunization regimen wherein the pseudotyped lentiviral vector particle elicits lung-resident memory CD8⁺ T cells (Trm) and/or effector CD8⁺ T cells (Tc1) specific to Spike and able to produce Interferon-gamma (IFN-γ)/Tumor Necrosis Factor (TNF)/Interleukin-2 (IL-2) cytokines. In a particular embodiment, the pseudotyped lentiviral vector particle encoding a S protein of a ARS-CoV-2 or a derivative thereof for use according to the invention is used in an immunization regimen wherein the subject has a waning immunity from week 12 after the first injection of the initial vaccination with a vaccine composition against SARS-CoV-2 infection or disease selected from the group consisting of a protein, an mRNA, an adenovirus, an inactivated virus and a protein subunit vaccine composition against SARS-CoV-2 infection or disease, in particular a protein- or an mRNA-based vaccine against SARS-CoV-2 infection or disease or post SARS-CoV-2 disease recovery, in particular post-COVID-19 recovery.

In pre-clinical results in mice, the inventors showed that 4 to 5 months after the last administration of an mRNA-based vaccine expressing the Spike antigen, there was no longer protective immunity against SARS-CoV-2 (Vesin et al., 2022, Mol Ther 30, 2984-2997). Furthermore, it is now well established that the level of neutralizing anti-Spike antibodies is significantly reduced in the serum of vaccinated individuals 3 to 10 months after the last administration of an mRNA-based vaccine expressing the Spike antigen (Decru et al., 2022, Front Immunol. 13, 909910).

It is also widely established that after pre-exposure of the immune system to an antigen, including SARS-CoV-2 Spike, in the context of vaccination—regardless of vaccination strategy—or in the context of infection, a memory immunity is generally induced in the B and T cell compartments (Valyi-Nagy et al., 2022, Int J Mol Sci, 23.10.3390). As far as can be currently assessed, in the case of anti-Spike immunity, induced by vaccination or infection, such memory immunity is expected to last on average at least one year (Gallais et al., 2021, EBioMedicine, 71, 103561). This memory immunity will be boostable, at least until one year after the last administration of an mRNA vaccine.

In a particular embodiment, the pseudotyped lentiviral vector particle encoding a S protein of a SARS-CoV-2 or a derivative thereof for use according to the invention is administered in a subject according to the invention as an intranasal mucosal boost or target immunization at least 3 months, in particular from 3 to 24 months, preferably from 3 to 12 months, after the last contact with SARS-CoV-2 or administration of a vaccine composition against SARS-CoV-2 infection or disease selected from the group consisting of a protein, an mRNA, an adenovirus, an inactivated virus and a protein subunit vaccine composition against SARS-CoV-2 infection or disease, in particular a protein or an mRNA vaccine composition against SARS-CoV-2 infection or disease.

In a particular embodiment, the pseudotyped lentiviral vector particle encoding a S protein of a SARS-CoV-2 or a derivative thereof for use according to the invention is formulated as a liquid composition or a dry powder for an administration as intranasal aerosols, intranasal drops or intranasal insufflations. In a particular embodiment, the pseudotyped lentiviral vector particle encoding a S protein of a SARS-CoV-2 or a derivative thereof for use according to the invention, is used in an immunization regimen wherein the administration regimen comprises administration of one or more dosage form(s) of the pseudotyped lentiviral vector particle wherein the dose of each dosage form is from 10⁷ to 10⁹ Transduction Unit (TU).

According to another aspect the invention also concerns an immunogenic composition comprising a pseudotyped lentiviral vector particle encoding a S protein of a SARS-CoV-2 or a derivative thereof and a pharmaceutically acceptable carrier, wherein the pseudotyped derivative of the S protein of SARS-CoV-2 comprises at least nine amino acid mutations including (i) a mutation of the lysine residue to the asparagine residue at position 417 of the amino acid sequence of SEQ ID NO: 1 (K417N), (ii) a mutation of the glutamic acid residue to the alanine residue at position 484 of the amino acid sequence of SEQ ID NO: 1 (E484A), (iii) a mutation of the asparagine residue to the tyrosine residue at position 501 of the amino acid sequence of SEQ ID NO: 1 (N501Y), (iv) a mutation of the lysine residue to the proline residue at position 986 of the amino acid sequence of SEQ ID NO: 1 (K986P), (v) a mutation of the valine residue to the proline residue at position 987 of the amino acid sequence of SEQ ID NO: 1 (V987P), (vi) a mutation of the glycine residue to the serine residue at position 446 of the amino acid sequence of SEQ ID NO: 1 (G446S), (vii) a mutation of the threonine residue to the lysine residue at position 478 of the amino acid sequence of SEQ ID NO: 1 (T478K), (viii) a mutation of the glutamine residue to the arginine residue at position 493 of the amino acid sequence of SEQ ID NO: 1 (Q493R) and (ix) a mutation of the glutamine residue to the arginine residue at position 498 of the amino acid sequence of SEQ ID NO: 1 (Q498R).

This immunogenic composition may be such that the pseudotyped lentiviral vector particle encodes mutated S protein of SARS-CoV-2 the amino acid sequence of which is SEQ ID NO: 18.

According to another embodiment, this immunogenic composition may be such that the pseudotyped lentiviral vector particle encodes mutated S protein of SARS-CoV-2 the amino acid sequence of which is SEQ ID NO: 24 or SEQ ID NO: 26.

According to a further embodiment, the immunogenic composition is formulated for intranasal administration as disclosed in the embodiments herein.

The invention also concerns kits suitable for use in practicing a use or a method disclosed herein. In some embodiments the kit comprises a dosage form for administration to the upper respiratory tract of a subject of the pseudotyped lentiviral vector particle encoding a SARS-CoV-2 S protein or a derivative thereof according to this disclosure, and an applicator. In some embodiments the applicator is an applicator for aerosol inhalation. In some embodiments the applicator is an applicator for nasal instillation. In some embodiments the applicator is an applicator for nasal insufflation. Suitable examples of each are known in the art and may be used.

Preparation of recombinant LV particles is known in the art, including to obtain non-integrative, non-replicative recombinant LV particles. In particular reference is made to the disclosure in Ku M W, et al. Cell Host Microbe, 29(2), 236-249 e236, 2021; Ku M W et al, EMBO Mol Med, e14459, 2021, Ku M W, Charneau P, Majlessi L. Expert Rev Vaccines, 1-16, 2021). Polynucleotide constructs may be adapted with the sequence encoding the selected Spike protein or derivative thereof.

In some embodiments, the lentiviral vector particle comprises HIV-1 Gag and Pol proteins. In some embodiments, the lentiviral vector particle comprises subtype D, especially HIV-1_{NDK}, Gag and Pol proteins.

According to some embodiments, the lentivector particles are obtained in a host cell transformed with a DNA plasmid.

Such a DNA plasmid can comprise:
bacterial origin of replication (ex: pUC ori);
antibiotic resistance gene (ex: AmpiR or KanR) for selection; and more particularly:
a lentiviral vector comprising at least one nucleic acid encoding a SARS-CoV-2 S protein or a derivative thereof, transcriptionally linked to a promoter, for example a CMV promoter.

Such a method allows producing a recombinant vector particle for use according to the invention, comprising the following steps of:
i) transfecting a suitable host cell with a lentiviral vector;
ii) transfecting said host cell with a packaging plasmid vector, containing viral DNA sequences encoding at least structural and polymerase (+ integrase) activities of a retrovirus (preferably lentivirus); Such packaging plasmids are described in the art (Dull et al., 1998, J Virol, 72(11):8463-71; Zufferey et al., 1998, J Virol 72(12):9873-80);
iii) culturing said transfected host cell in order to obtain expression and packaging of said lentiviral vector into lentiviral vector particles; and
iv) harvesting the lentiviral vector particles resulting from the expression and packaging of step iii) in said cultured host cells.

An appropriate host cell is preferably a human cultured cell line as, for example, a HEK cell line, such as a HEK293T line.

Alternatively, the method for producing the vector particle is carried out in a host cell, which genome has been stably transformed with one or more of the following components: a lentiviral vector DNA sequence, the packaging genes, and the envelope gene. Such a DNA sequence may be regarded as being similar to a proviral vector according to the invention, comprising an additional promoter to allow the transcription of the vector sequence and improve the particle production rate.

In a preferred embodiment, the host cell is further modified to be able to produce viral particle in a culture medium in a continuous manner, without the entire cells swelling or dying. One may refer to Strang et al., 2005, J Virol 79(3):1165-71; Relander et al., 2005, Mol Ther 11(3):452-9; Stewart et al., 2009, Gene Ther, 16(6):805-14; and Stuart et al., 2011, Hum gene Ther, with respect to such techniques for producing viral particles.

The lentiviral particle vectors can comprise the following elements, as previously defined:
cPPT/CTS polynucleotide sequence; and
a nucleic acid encoding a CAR under control of a β2m or Major Histocompatibility Complex classe I (MHC-1) promoter, and optionally one of the additional elements described above.

Other features and advantages of the invention will be apparent from the examples which follow and will also be illustrated in the figures.

### BRIEF DESCRIPTION OF THE FIGURES

FIG. 1: Down-selection of a S_{CoV-2} variant with the highest potential to induce cross sero-neutralizing antibodies. (A) Timeline of prime-boost vaccination with LV::S_{Alpha}, LV::S_{Beta} or LV::S_{Gamma} and (cross) sero-neutralization assays in C57BL/6 mice (n=4-5/group). (B) Half maximal Effective Concentration (EC50) of neutralizing activity of sera from vaccinated mice was evaluated before and after the boost, against pseudo-viruses carrying S_{CoV-2} from D614G, Alpha, Beta or Gamma variants. (C) EC50 of sera from C57BL/6 mice, vaccinated following the regimen detailed in (A) with LV encoding for S_{D614G}, either WT or carrying the K⁹⁸⁶P-V⁹⁸⁷P substitutions in the S2 domain. EC50 was evaluated before and after the boost, as indicated in (B).

FIG. 2: Anti-S_{CoV-2} humoral responses in mRNA-1273-vaccinated mice which were further intranasally boosted with LV::S_{Beta-2P}. (A) Timeline of mRNA-1273 intramuscular (i.m.)-i.m. prime-boost vaccination in C57BL/6 mice which were later immunized i.n. by escalating doses of LV::S_{Beta-2P} (n=4-5/group) and the (cross) sero-neutralization follow-up. (B) Serum EC50 determined at the indicated time points against pseudo-viruses carrying S_{CoV-2} from D614G, Alpha, Beta, Gamma, Delta or Delta+ variants. (C) Anti-S_{CoV-2} IgG or IgA in the sera two weeks after late LV::S_{Beta-2P} i.n. or mRNA-1273 (Moderna) i.m. boost. Statistical significance was determined by Mann-Whitney test (*=p<0.05, **=p<0.01, ***=p<0.001).

FIG. 3: Lung resident memory B-cell subsets in mRNA-1273-vaccinated mice which were further intranasally boosted with LV::S_{Beta-2P}. The mice are those detailed in FIG. 2. Mucosal immune cells were studied two weeks after LV::S_{Beta-2P} i.n. boost. (A) Cytometric gating strategy to detect lung B resident memory cells, and (B) percentages of Brm among surface IgM/IgD⁻ B cells in various groups.

FIG. 4: Systemic CD8⁺ T-cell responses to S_{CoV-2} in mRNA-1273-vaccinated mice which were further intranasally boosted with LV::S_{Beta-2P}. The mice are those detailed in FIG. 2. T-splenocyte responses were evaluated two weeks after LV::S_{Beta-2P} i.n. boost by IFN-γ ELISPOT after stimulation with S:256-275, S:536-550 or S:576-590 synthetic 15-mer peptides encompassing S_{CoV-2} MHC-1-restricted epitopes, pertinent in C57BL/6 (H-2^{b}) mice. Statistical significance was evaluated by Mann-Whitney test (*=p<0.05).

FIG. 5: Mucosal CD8⁺ T-cell responses to S_{CoV-2} in mRNA-1273-vaccinated mice which were further intranasally boosted with LV::S_{Beta-2P}. The mice are those detailed in FIG. 2. (A) Representative IFN-γ response by lung CD8⁺ T cells detected by IntraCellular Cytokine Staining (ICS) after in vitro stimulation with a pool of S:256-275, S:536-550 and S:576-590 peptides. Cells are gated on alive CD45⁺ CD8⁺ T cells.

FIG. 6. Lung resident memory T-cell subset in mRNA-1273-vaccinated mice which were further intranasally boosted with LV::S_{Beta-2P}. The mice are those detailed in FIGS. 2 and 3. Mucosal immune cells were studied two weeks after LV::S_{Beta-2P} i.n. boost. (A) Cytometric gating strategy to detect lung CD8+T resident memory cells (Trm, CD44⁺CD62L⁻CD69⁺CD103⁺), and (B) percentages of Trm among CD8⁺CD44⁺ T-cells in various groups.

FIG. 7. Full protective potential of a late LV::S_{Beta-2P} i.n. boost in mRNA-1273-primed and-boosted mice. (A) Timeline of mRNA-1273 i.m.-i.m. prime-boost vaccination in C57BL/6 mice which were later immunized i.n. with the sub-optimal dose of 1×10⁸ TU/mouse of LV::S_{Beta-2P} (n=4-5/group), pre-treated i.n. with Ad5::hACE-2 4 days before i.n. challenge with 0.3×10⁵ TCID₅₀ (Tissue Culture Infectious Dose of 50%) of SARS-CoV-2 Delta variant. (B) Comparative quantitation of hACE-2 mRNA in the lungs of different groups of mice pre-treated i.n. with Ad5::hACE-2. (C) Viral RNA contents, evaluated by conventional E_{CoV-2}-specific (top) or sub-genomic Esg-specific (bottom) qRT-PCR at 3 dpi. The bottom line indicates the detection limits. Statistical significance was evaluated by Mann-Whitney test (*=p<0.05, **=p<0.01).

FIG. 8. Anti-S_{CoV-2} humoral responses in mRNA-1273-vaccinated mice which were further intranasally boosted with LV::S_{Beta-2P}. (A) Follow-up of anti-S_{CoV-2} (left) and anti-RBD (right) IgG in the sera of mice initially primed and boosted i.m. with mRNA-1273. (B) Anti-S_{CoV-2} IgG (top), anti-RBD IgG (middle), and anti-S_{CoV-2} IgA (bottom) in the total lung extracts of mice initially primed and boosted i.m. with mRNA-1273 and then boosted later with a third i.m. dose of mRNA-1273 or an i.n. boost of LV::S_{Beta-2P}.

FIG. 9. Absence of mucosal CD8⁺ Tc2 responses to S_{CoV-2} in mRNA-1273-vaccinated mice which were further intranasally boosted with LV::S_{Beta-2P}. The mice are those detailed in FIG. 2. Absence of IL-4, IL-5, IL-10 and IL-13 production by lung CD8⁺ T cells after in vitro stimulation with a pool of S:256-275, S:536-550 and S:576-590 peptides, studied by ICS in parallel to the assay performed to detect IFN-γ/TNF/IL-2 (see FIG. 5). Cells are gated on alive CD45⁺ CD8⁺ T cells.

FIG. 10. Maps of plasmids used for production of LV encoding (A) S_{D614G-2P}, (B) S_{Alpha-2P}, (C) S_{Gamma-2P}, (D) S_{Delta-2P}, (E) S_{Beta-2P} and (F) S_{Omicron-2P} antigens.

FIG. 11. Amino acid sequence of Spike from (A) Omicron BA.1 (top) or (B) Omicron BA 4/5 (bottom) sub-variants. Sequences indicated in bold and underlined are murine MHC-1-restricted T-cell epitopes in H-2^{b} mice (Ku M W, et al. EMBO Mol Med, e14459, 2021). Sequences highlighted in gray are MHC-I or -II-restricted human T-cell epitopes identified in HHD-DR1 MHC-humanized mice.

FIG. 12. Humoral immunity in hamsters immunized i.m. with various LV::S. (A) Schematic representation of LV encoding S_{CoV-2} proteins from either ancestral WA1 or Beta SARS-CoV-2 strain. Codon-optimized sequences encoding S_{CoV-2} were cloned into the pFLAP lentiviral vector plasmid, under the control of human P_{CMvie} promoter; RRE, rev response element; cPPT, central polypurine tract. The LV::S_{WA1} includes the entire sequence of S_{CoV-2}. RBD, S1/S2, S2′ cleavage sites, 675^{QTQTNSPRRAR}685 sequence encompassing RRAR furin cleavage site of SEQ ID NO: 27, and K⁹⁸⁶P, V⁹⁸⁷P, K⁴¹⁷N, E⁴⁸⁴K and N⁵⁰¹Y substitutions are indicated. (B) Western blot analysis to detect expression of S_{WA1}, S_{WA1-2P}, S_{WA1-ΔF-2P}, and S_{Beta2-P} in LV-transduced 293T cells. Total cell lysates were analyzed under non-reduced conditions using an anti-S2 rabbit polyclonal antibody. LV::GFP was included as negative control. Full length Spike (S) and S2 subunit are indicated. (C) Syrian golden hamsters (n=4/group) were immunized i.m. with 1×10⁸ TU of LV::S_{WA1}, LV::S_{WA1-2P}, LV::S_{WA1ΔF-2P} or LV::S_{Beta-2P}. Five weeks later, serum anti-S_{WA1} responses expressed as mean endpoint dilution titers, were determined by ELISA. Errors bars represent the standard error of the mean (SEM). The statistical significance of differences was determined by Kruskal-Wallis test followed by Dunn's multiple comparisons test and were found not significant. Dotted lines indicate the limit of detection (LOD).

FIG. 13. Humoral immunity in hamsters following LV::S administration. (A) Timeline of the LV::S prime-boost vaccination regimen and WA1 SARS-CoV-2 challenge in hamsters (n=6/group). (B) Serum anti-S_{WA1} or -RBD_{WA1} IgG responses expressed as mean endpoint dilution titers, determined by ELISA. (C) Neutralizing activity (EC50) of sera, taken prior the WA1 SARS-CoV-2 challenge, or of lung homogenates, taken at 4 dpi as determined by use of pseudoviruses harboring S_{CoV-2} from D614G SARS-CoV-2 variant. Data are presented as mean±SEM. Asterisks indicate the significance of differences between the groups. p-values were determined by using Kruskal-Wallis tests followed by Dunn's multiple comparisons tests; *p<0.05, **p<0.01. Only significant differences are shown. Dotted lines indicate the LOD.

FIG. 14. Single i.n. LV::S injection fully protects hamsters against WA1 SARS-CoV-2. Hamsters are those described in the legend to the FIG. 13. (A) Lung viral loads quantitated by total E (left) or Esg qRT-PCR (right) at 4 dpi. Bars represent geometric means. (B) Percentages of weight loss in LV::S- or LV ctrl-vaccinated hamsters at 4 dpi. (C) Expression of inflammatory cytokines in lung tissues after challenge. The heatmap recapitulates relative log₂ fold changes in the expression of inflammation-related mediators in LV::S vaccinated or LV ctrl-administered individuals, as analyzed at 4 dpi by use of RNA extracted from total lung homogenates and normalized versus samples from untreated controls. Six individual hamsters per group are shown in the heatmap. Statistical differences between LV:: S and LV ctrl groups were determined by Kruskal-Wallis test followed by Dunn's multiple comparisons test and are indicated by asterisks; *p<0.05; **p<0.01; ***p<0.001. Comparisons were made between vaccinated groups and LV ctrl.

FIG. 15. Single i.n. LV:: S injection largely reduced lung histopathogy. (A) Lung histological H&E analysis, as studied at 4 dpi. Heatmap recapitulating the histological scores, for: 1) inflammation score and 2) interstitial syndrome. (B) Representative alveolo-interstitial syndrome and (C) severe inflammation in an LV ctrl-injected and infected hamster. Here the structure of the organ is largely obliterated, while remnants of alveolar spaces and bronchiolar lumens can be seen. (D-F) bronchiolar lesions in LV ctrl-immunized animals. Shown are epithelial cells and cell debris in the bronchiolar lumen (black arrows) (D), papillary projections of the bronchiolar epithelium into the lumen (star) (E) and degenerative lesions with effacement of the epithelium (green arrow) (F). (G) Mild alveolar infiltration in a vaccinated hamster. Some of the alveoli (arrow) are partially or completely filled with cells and an eosinophilic exudate. (H) Representative N_{CoV-2}-specific IHC image performed on lungs of SARS-CoV-2-infected hamsters. Lower panels show enlarged views from upper panels. Scale bars are 1 mm for upper panels and 25 μm for lower panels.

FIG. 16. Decreased SARSCoV-2 omicron infectious virus in lungs and nasal turbinates by i.n administration of a single or booster dose of LV::S_{Beta-2P}. (A) Timeline of single or prime-boost vaccination and Omicron SARS-CoV-2 challenge. Hamsters (n=4-5/group) were primed i.n. or i.m. with 1×10⁸ TU of LV::S_{Beta-2P}. Three weeks later, some of them were boosted i.n. with the same amount of LV::S_{Beta-2P} or LV ctrl. Serum samples were collected at wks 3 and 7 for serological analyses. (B) Serum or anti-S_{Omicron} (upper panels) or -RBD_{Omicron} (lower panels) IgG responses, expressed as mean endpoint dilution titers, determined by ELISA. Data are presented as mean±SEM. Percentages of weight loss in LV::S_{Beta-2P}-, or LV ctrl-vaccinated hamsters following challenge (C) and at 4 dpi (D). (E) Lung and (F) NT viral loads were quantitated by Esg qRT-PCR at 4 dpi. Bars represent geometric means. Statistical differences were determined by Kruskal-Wallis tests followed by Dunn's multiple comparisons test and are indicated by asterisks. *p<0.05, **p<0.01, ***p<0.001. Dotted lines indicate the LOD.

FIG. 17. Immunodetection of the NCoV-2 antigen performed on lungs of Omicron SARS-CoV-2-infected hamsters. Hamsters are those described in FIG. 16. (A) One example of each vaccination regimen is shown at low magnification. Solid arrows denote foci of inflammatory infiltrates, and dotted arrows areas where the immunodetection signal is discernable even at this low magnification. (B) The close-up views depict the concentration of viral antigen (brown) within the inflammatory foci (bottom), while areas harboring no or little inflammation (top) display only scarce staining.

FIG. 18. Robust humoral responses in hamsters vaccinated by LV::S_{WA1-2P} or LV::S_{Beta-2P} prime (i.m.)-LV::S_{Beta-2P} boost (i.n.). (A) Timeline of prime-boost vaccination. Hamsters (n=4/group) were primed i.m. with 1×10⁸ TU of LV::S_{WA1-2P} or LV::S_{Beta-2P}. Five weeks later, hamsters were boosted i.n. with the same amount of LV::S_{Beta-2P}. (B) Serum anti-SWA1 or -RBD_{WA1} (left panels) or anti-S_{Omicron} or -RBD_{Omicron} (right panels) IgG responses, expressed as mean endpoint dilution titers ±SEM, determined by ELISA. Statistical differences are indicated by asterisks. *p<0.05.

FIG. 19. Anti-S_{CoV-2} antibody imprinting in hamsters vaccinated by LV::S prime (i.m.)-LV::S_{Beta-2P} boost (i.n.). Hamsters are those described in the legend to the FIG. 18. (A-C) EC50 determined by use of pseudo-viruses carrying S_{CoV-2} from D614G, Alpha, Beta, Gamma, Delta or Omicron variants. EC50 (A) 5 wks post prime in sera or 2 wks post boost in sera (B) and in lung homogenates (C). Data are expressed as the geometric mean EC50. Statistical significances were analyzed using two-way ANOVA followed by Sidak's multiple comparisons test; **p<0.01; ****p<0.0001. Dotted lines indicate the lower limit of detection (LOD).

FIG. 20. Full protective capacity of LV::S_{Beta-2P} used in a prime (i.m.) boost (i.n.) regimen against Omicron variant. (A) Timeline of prime-boost vaccination and i.n. challenge with 0.3×10⁵ TCID₅₀ of SARS-CoV-2 Omicron variant in B6.K18-hACE2^{IP-THV} transgenic mice (n=5/group). (B) Lung viral RNA contents, evaluated by sub-genomic Esg-specific qRT-PCR at 5 dpi. Bottom lines indicate the detection limits. Statistical significance was evaluated by Mann-Whitney test (*=p<0.05, **=p<0.01).

The LV-based strategy, which is highly productive, not only in inducing humoral responses but also and particularly in establishing high quality and memory T-cell responses (Ku M W, et al. Commun Biol, 4(1), 713, 2021), is a favorable platform for a heterologous boost, even if it is also largely efficacious by its own as a primary COVID-19 vaccine candidate (Ku M W, et al. Cell Host Microbe, 29(2), 236-249 e236, 2021; Ku M W, et al. EMBO Mol Med, e14459, 2021). Furthermore, and importantly, LV is non-cytopathic, non-replicative and scarcely inflammatory and thus can be used to perform non-invasive i.n. boost, to efficaciously induce sterilizing mucosal immunity which protects the respiratory system, as well as the CNS (Ku M W, et al. Cell Host Microbe, 29(2), 236-249 e236, 2021; Ku M W, et al. EMBO Mol Med, e14459, 2021). The i.n. route of vaccination has been shown by several teams to be the most efficacious route at reducing viral contents in nasal swabs and nasal olfactory neuroepithelium (Bricker T L, Cell Rep, 36(3), 109400, 2021; Hassan A O, et al. Cell Rep Med, 2(4), 100230, 2021). The inventors thus hypothesized that i.n. vaccination could contribute efficaciously in blocking/reducing the respiratory chain of SARS-CoV-2 transmission.

Another major advantage of LV-based immunization is the induction of strong T-cell immune responses with high cross-reactivity of T-cell epitopes from Spike of diverse VOCs. Therefore, when the neutralizing antibody fails or wanes, the T-cell arm remains largely protective, as the inventors recently described in antibody-deficient, B-cell compromised μMT KO mice (Ku M W, et al. EMBO Mol Med, e14459, 2021). This property is relative to a high-quality and long-lasting T-cell immunity induced against multiple preserved T-cell epitopes, despite the mutation accumulated in the Spike of the emerging VOCs (Ku M W, et al. EMBO Mol Med, e14459, 2021).

In the present invention, the inventors first down-selected S_{Beta} antigen which induced the greatest neutralization breadth against the VOCs and designed a non-integrating LV encoding a stabilized version of this antigen. In mRNA-1273-primed and -boosted mice with waning (cross) sero-neutralization capacity, the inventors used escalating doses of LV::S_{Beta-2P} in i.n. boost. The inventors demonstrated a dose-dependent increase in anti-S_{CoV-2} IgG and IgA titers, and a broadened sero-neutralization potential both in the sera and lung homogenates against VOCs. No anti-S_{CoV-2} IgA was detected in the lungs of mice injected i.m. with the third dose of mRNA-1273. Increasing proportions of lung surface IgM/D⁻ B cells with CD38⁺ CD73⁺ CD62L⁺ CD69⁺ CD80⁺ phenotype, which have been associated with resident memory characteristic, were detected in a dose-dependent manner, in these mice. No such increases in this B-cell subset were detected in their counterparts which received an additional mRNA-1273 dose via i.m.

Spike-specific, effector lung CD8⁺ Tc1 cells were largely detected in the initially mRNA-1273-primed and boosted mice which received a late i.n. LV::S_{Beta-2P} boost. These lung CD8⁺ T cells did not display Tc2 phenotype. Increasing proportions of lung CD8⁺ CD44⁺ CD69⁺ CD103⁺ Trm were also detected, in a dose-dependent manner, only in LV::S_{Beta-2P} i.n. boosted mice, but not in their counterparts boosted i.m. with mRNA-1273. The systemic CD8⁺ T-cell responses against various immunogenic regions of S_{CoV-2} were also increased with 1×10⁸ or 1×10⁹ TU of LV::S_{Beta-2P} i.n. boost in initially mRNA-1273-primed and boosted mice. The highest i.n. dose of LV::S_{Beta-2P} was comparable to the additional i.m. dose of mRNA-1273. The fact that the i.n. administration of LV::S_{Beta-2P} has a boost effect on the systemic T-cell immunity represents another advantage of this vaccination regimen.

Evaluation of the protective potential of the lungs of mice primed and boosted with mRNA-1273, without additional boosting, showed that 20 wks after the first injection of mRNA-1273, there was no detectable protective capacity left against the Delta variant of SARS-CoV-2. In these mice, an i.n. booster injection of suboptimal dose, i.e., 1×10⁸ TU of LV::S_{Beta-2P} completely inhibited SARS-CoV-2 replication in the lungs. A third late i.m. booster injection of mRNA-1273 reduced SARS-CoV-2 RNA content in the lungs in a similar manner, but did not completely inhibit viral replication in all mice.

The lack of protective potential against the Delta variant 4 months after prime-boost to mRNA-1273 could be explained by the hypothesis that adaptive immune memory is likely to be located in secondary lymphoid organs, i.e., distant anatomical sites far from the respiratory tract. In this context, the extraordinarily rapid replication of new VOCs, such as Delta and Omicron variants, at the site of the infection would not allow sufficient time for reactivation of immune memory and early enough to prevent local mucosal infection, replication and viral transmission.

In the present invention, the inventors provided numerous evidences that LV:: S_{Beta-2P} i.n. boost can be used to induce robust systemic and mucosal adaptive immunity, to broaden the specificity of the protective response. The LV::S_{Beta-2P} i.n. boost strengthen the intensity, broaden the VOC cross-recognition, and targets B- and T-cell immune responses to the principal entry point of SARS-CoV-2 to the mucosal respiratory of the host organism and avoid the infection of main anatomical sites. A phase/IIa clinical trial is currently in preparation for the use of i.n. boost by LV::S_{Beta-2P} in previously vaccinated persons or in COVID-convalescents.

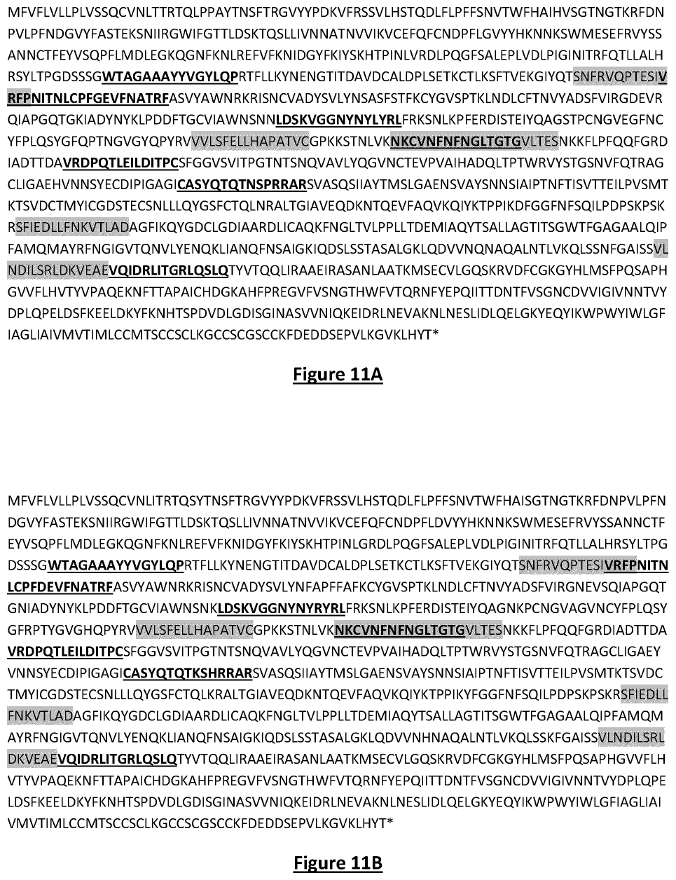

The inventors established that B-cell independent and antigen-specific T-cell immunity played a major role in LV-mediated protection against SARS-CoV-2 infection (Ku M W, et al. EMBO Mol Med, e14459, 2021). This was consistent with the strong T-cell responses induced by LV::S_{Beta-2P} at the systemic level and in the lungs, detectable in vaccinated mice (Vesin, B., et al. Mol Ther 30, 2984-2997, 2022). Importantly, all or the vast majority of murine and human T-cell epitopes, identified on the ancestral S_{CoV-2} sequence and included in SBeta-2P, were preserved in the mutated Spike from the emerging variants, including Omicron BA.1 and BA.4/5 sub-variants (FIG. 11).

These observations indicated the strong capability of LV expressing the Spike protein of SARS-CoV-2 especially LV::S_{Beta-2P} at inducing full protection against not only the ancestral but also against the recently emerging SARS-CoV-2 variants by eliciting robust T-cell responses.

In contrast to the B-cell epitopes which were targets of neutralizing antibodies, the so far identified T-cell epitopes had not been impacted or were barely impacted by mutations accumulated in the S_{CoV-2} of the emerging variants (FIG. 11).

This observation indicates why the LV platform, which induces strong T-cell immunity, has an invariable and complete protective capacity against the newly emerging variants.

### Materials and Method

### Mice Immunization and SARS-CoV-2 Infection

Female C57BL/6JRj mice were purchased from Janvier (Le Genest Saint Isle, France), housed in individually-ventilated cages under specific pathogen-free conditions at the Institut Pasteur animal facilities and used at the age of 7 wks. Mice were immunized i.m. with 1 μg/mouse of mRNA-1273 (Moderna) vaccine. For i.n. injections with LV, mice were anesthetized by i.p. injection of Ketamine (Imalgene, 80255 mg/kg) and Xylazine (Rompun, 5 mg/kg). For protection experiments against SARS-CoV-2, mice were transferred into filtered cages in isolator. Four days before SARS-CoV-2 inoculation, mice were pretreated with 3×10⁸ IGU of Ad5::hACE2 as previously described (Ku M W, et al. Cell Host Microbe, 29(2), 236-249 e236, 2021). Mice were then transferred into a level 3 biosafety cabinet and inoculated i.n. with 0.3×10⁵ TCID50 of the Delta SARS-CoV-2 clinical isolate (Lescure F X, et al. Lancet Infect Dis, 20(6), 697-706, 2020) contained in 20 μl. Mice were then housed in filtered cages in an isolator in BioSafety Level 3 animal facilities. The organs recovered from the infected animals were manipulated according to the approved standard procedures of these facilities.

### Ethical Approval of Animal Experimentation.

Experimentation on animals was performed in accordance with the European and French guidelines (Directive 86/609/CEE and Decree 87-848 of 19 Oct. 1987) subsequent to approval by the Institut Pasteur Safety, Animal Care and Use Committee, protocol agreement delivered by local ethical committee (CETEA #DAP20007, CETEA #DAP200058, and Ministry of High Education and Research APAFIS #24627-2020031117362508 v1, APAFIS #28755-2020122110238379 v1).

### Construction and Production of Vaccinal LVs

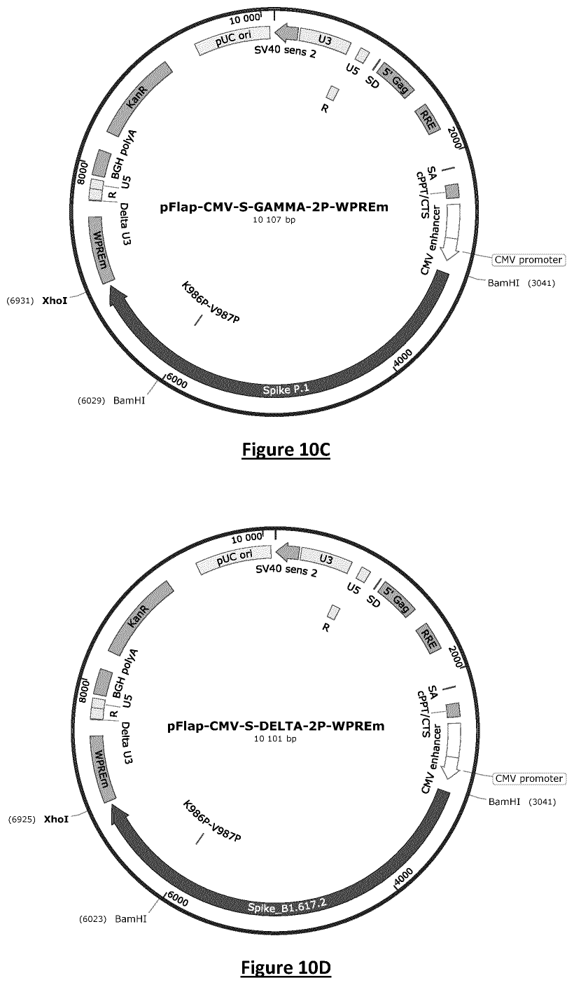

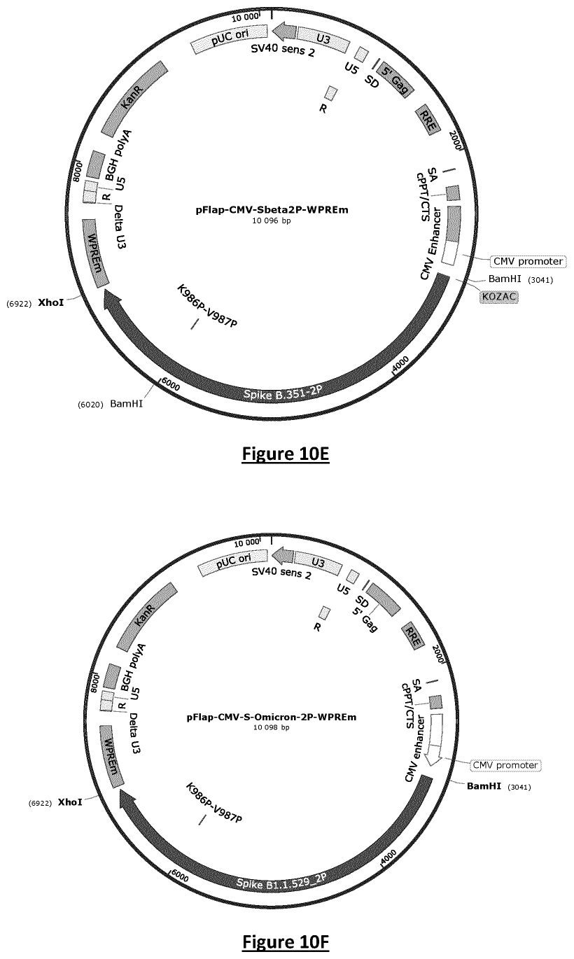

First, a codon-optimized sequence of Spike from the Ancestral, D614G, Alpha, Beta or Gamma VOCs were synthetized and inserted into the pMK-RQ_S-2019-nCoV_S501YV2 plasmid. The S sequence was then extracted by BamHI/XhoI digestion to be ligated into the pFlap lentiviral plasmid between the BamHI and XhoI restriction sites, located between the native human ieCMV promoter and the mutated atg starting codon of Woodchuck Posttranscriptional Regulatory Element (WPRE) sequence (See the plasmid maps, FIG. 10). To introduce the K⁹⁸⁶P-V⁹⁸⁷P “2P” double mutation in S_{D614G} or S_{Beta}, a directed mutagenesis was performed by use of Takara In-Fusion kit on the corresponding pFlap plasmids. Various pFlap-ieCMV-S-WPREm or pFlap-ieCMV-S_{2P}-WPREm plasmids were amplified and used to produce non-integrative vaccinal LV, as described elsewhere, (Ku M W, et al. Cell Host Microbe, 29(2), 236-249 e236, 2021; Ku M W, Charneau P, Majlessi L. Expert Rev Vaccines, 1-16, 2021).

### Analysis of Humoral and Systemic T-Cell Immunity

Anti-S_{CoV-2} IgG and IgA antibody titers were determined by ELISA by use of recombinant stabilized S_{CoV-2} or RBD fragment for coating. Neutralization potential of clarified and decomplemented sera or lung homogenates was quantitated by use of lentiviral particles pseudo-typed with S_{CoV-2} from diverse variants, as previously described (Ku M W, et al. Cell Host Microbe, 29(2), 236-249 e236, 2021; Sterlin D, et al. Sci Transl Med, 13(577), 2021).

T-splenocyte responses were quantitated by IFN-γ ELISPOT after in vitro stimulation with S:256-275, S:536-550 or S:576-590 synthetic 15-mer peptides which contain S_{CoV-2} MHC-I-restricted epitopes in H-2^{d} mice (Ku M W, et al. Cell Host Microbe, 29(2), 236-249 e236, 2021). Spots were quantified in a CTL Immunospot S6 ultimate-V Analyser by use of CTL Immunocapture 7.0.8.1 program.

### Phenotypic and Functional Cytometric Analysis of Lung Immune Cells

Enrichment and staining of lung immune cells were performed as detailed elsewhere (Ku M W, et al. Cell Host Microbe, 29(2), 236-249 e236, 2021; Ku M W, et al. EMBO Mol Med, e14459, 2021) after treatment with 400 U/ml type IV collagenase and DNase I (Roche) for a 30-minute incubation at 37° C. and homogenization by use of GentleMacs (Miltenyi Biotech). Cell suspensions were then filtered through 100 μm-pore filters, centrifuged at 1200 rpm and enriched on Ficoll gradient after 20 min centrifugation at 3000 rpm at RT, without brakes. The recovered cells were co-cultured with syngeneic bone-marrow derived dendritic cells loaded with a pool of A, B, C peptides, each at 1 μg/ml or negative control peptide at ×290 μg/ml. The following mixture was used to detect lung Tc1 cells: PerCP-Cy5.5-anti-CD3 (45-0031-82, eBioScience), eF450-anti-CD4 (48-0042-82, eBioScience) and APC-anti-CD8 (17-0081-82, eBioScience) for surface staining and BV650-anti-IFN-g (563854, BD), FITC-anti-TNF (554418, BD) and PE-anti-IL-2 (561061, BD) for intracellular staining. The following mixture was used to detect lung Tc2 cells: PerCP-Cy5.5-anti-CD3 (45-0031-82, eBioScience), eF450-anti-CD4 (48-0042-82, eBioScience), BV711-anti-CD8 (563046, BD Biosciences), for surface staining and BV605-anti-IL-4 (504125, BioLegend Europe BV), APC-anti-IL-5 (504306, BioLegend Europe BV), FITC-anti-IL-10 (505006, BioLegend Europe BV), PE-anti-IL-13 (12-7133-81, eBioScience) for intracellular staining. The intracellular staining was performed by use of the Fix Perm kit (BD), following the manufacturer's protocol. Dead cells were excluded by use of Near IR Live/Dead (Invitrogen). Staining was performed in the presence of FcγII/III receptor blocking anti-CD16/CD32 (BD).

To identify lung resident memory CD8⁺ T-cell subsets, a mixture of PerCP-Vio700-anti-CD3 (130-119-656, Miltenyi Biotec), PECy7-CD4 (552775, BD Biosciences), BV510-anti-CD8 (100752, BioLegend), PE-anti-CD62L (553151, BD Biosciences), APC-anti-CD69 (560689, BD Biosciences), APC-Cy7-anti-CD44 (560568, BD Biosciences), FITC-anti-CD103 (11-1031-82, eBiosciences) and yellow Live/Dead (Invitrogen) was used. Lung B cells were studied by surface staining with a mixture of PerCP Vio700-anti-IgM (130-106-012, Miltenyi), and PerCP Vio700-anti-IgD (130-103-797, Miltenyi), APC-H7-anti-CD19 (560143, BD Biosciences), PE-anti-CD38 (102708, BioLegend Europe BV), PE-Cy7-anti-CD62L (ab25569, AbCam), BV711-anti-CD69 (740664, BD Biosciences), BV421-anti-CD73 (127217, BioLegend Europe BV), FITC-anti-CD80 (104705, BioLegend Europe BV) and yellow Live/Dead (Invitrogen).

Cells were incubated with appropriate mixtures for 25 minutes at 4° C., washed in PBS containing 3% FCS and fixed with Paraformaldehyde 4% after an overnight incubation at 4° C. Samples were acquired in an Attune NxT cytometer (Invitrogen) and data analyzed by FlowJo software (Treestar, OR, USA).

### Determination of Viral RNA Content in the Organs

Organs from mice were removed and immediately frozen at −80° C. on dry ice. RNA from circulating SARS-CoV-2 was prepared from lungs as described elsewhere (Ku M W, et al. Cell Host Microbe, 29(2), 236-249 e236, 2021). Lung homogenates were prepared by thawing and homogenizing in lysing matrix M (MP Biomedical) with 500 μl of PBS using a MP Biomedical Fastprep 24 Tissue Homogenizer. RNA was extracted from the supernatants of organ homogenates centrifuged during 10 min at 2000 g, using the Qiagen Rneasy kit, except that the neutralization step with AVL buffer/carrier RNA was omitted. The RNA samples were then used to determine viral RNA content by E-specific qRT-PCR. To determine viral RNA content by Esg-specific qRT-PCR, total RNA was prepared using lysing matrix D (MP Biomedical) containing 1 mL of TRIzol reagent (ThermoFisher) and homogenization at 30 s at 6.0 m/s twice using MP Biomedical Fastprep 24 Tissue Homogenizer. The quality of RNA samples was assessed by use of a Bioanalyzer 2100 (Agilent Technologies). Viral RNA contents were quantitated using a NanoDrop Spectrophotometer (Thermo Scientific NanoDrop). The RNA Integrity Number (RIN) was 7.5-10.0. SARS-CoV-2 E or E sub-genomic mRNA were quantitated following reverse transcription and real-time quantitative TaqMan® PCR, using SuperScript™ Ill Platinum One-Step qRT-PCR System (Invitrogen) and specific primers and probe (Eurofins), as recently described (Ku M W, et al. EMBO Mol Med, e14459, 2021).

### EXAMPLES

### Antigen Design and Down-Selection of a Lead Candidate

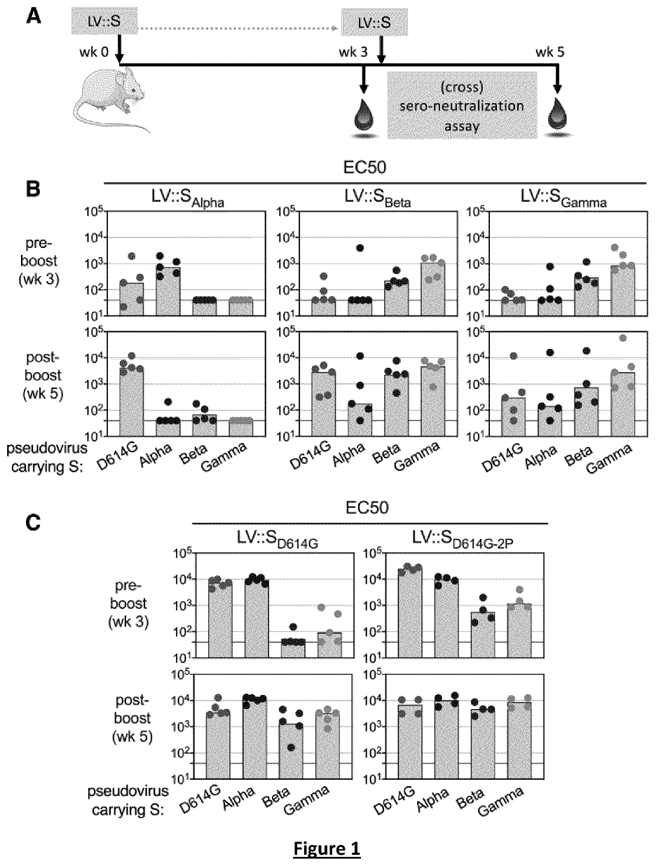

To select the most suitable S_{CoV-2} variant, which can consider the dynamics of virus spread of the known variants and able to induce the greatest neutralization breadth, the inventors generated LVs encoding the full length S_{CoV-2} from the Alpha, Beta or Gamma SARS-CoV-2 VOCs. C57BL/6 mice (n=5/group) were primed i.m. (wk 0) and boosted i.m. (wk 3) with 1×10⁸ TU/mouse of each individual LV and the (cross) neutralization potential of their sera was assessed before boost (wk 3) and after boost (wk 5) against pseudoviruses carrying various S_{CoV-2} (FIG. 1A). Immunization with LV::S_{Alpha} generated appropriate neutralization capacity against S_{D614G} and S_{Alpha} but not against S_{Beta} and S_{Gamma} (FIG. 1B). Between LV::S_{Beta} and LV::S_{Gamma}, the former generated the highest cross sero-neutralization potential against S_{D614G}, S_{Alpha} and S_{Gamma} variants. In accordance with previous observations using other vaccination strategies in the context of immunization with LV, the K⁹⁸⁶p-V⁹⁸⁷P substitutions in the S2 domain of SCoV-2 improved the (cross) sero-neutralization potential (FIG. 1C), which the most probably results from an extended half-life of S_{CoV-2-2P} (Walls A C, et al. Cell, 181(2), 281-292 e286, 2020).

Taken together these data allowed to down select S_{Beta-2P} as the best cross-reactive antigen candidate to be used in the context of LV (LV::S_{Beta-2P}) to strengthen the waning immunity previously induced by the first generation COVID-19 vaccines, like mRNA-1273.

Follow-Up of Humoral Immunity in mRNA-1273-Primed and -Boosted Mice and Effect of LV::S_{Beta-2P} i.n. Boost

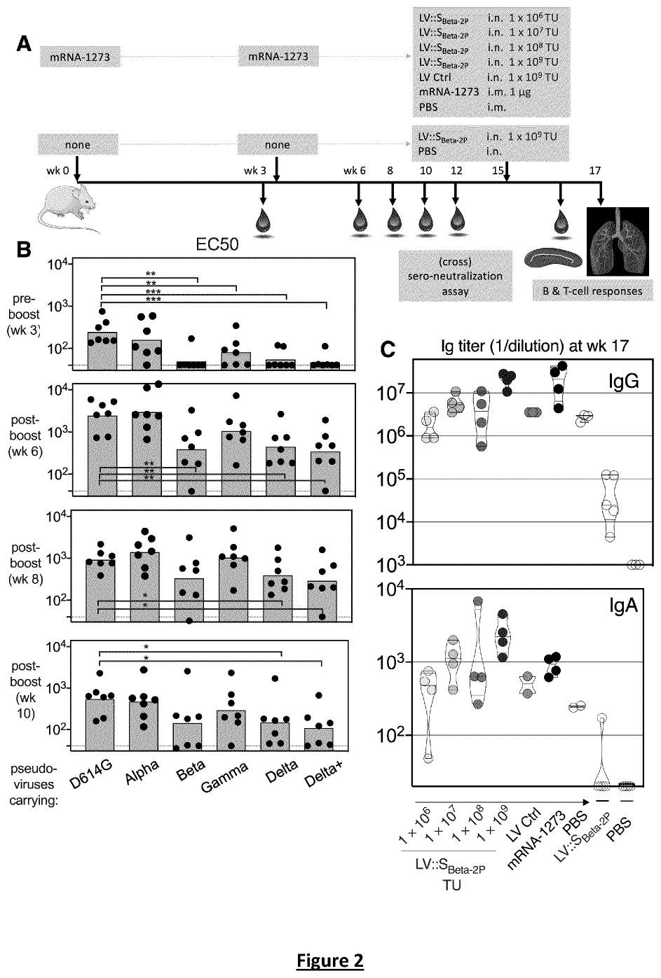

The inventors analyzed the potential of LV::S_{Beta-2P} i.n. boost vaccination to strengthen and broaden the immune responses in mice which have been initially primed and boosted with mRNA-1273 and in which the (cross) sero-neutralization potential is decreasing. C57BL/6 mice were primed i.m. at wk 0 and boosted i.m. at wk 3 with 1 μg/mouse of mRNA-1273, defined as the optimal dose of this vaccine in mice (Nature, 2020, Vol. 586, 567-571) (FIG. 2A). Longitudinal serological follow-up demonstrated that at 3 wks post prime, cross-neutralization activities against both S_{D614G} and S_{Alpha} were readily detectable (FIG. 2B). Cross sero-neutralization was also detectable, although to a lesser degree, against S_{Gamma}, but not against S_{Beta}, S_{Delta} or S_{Delta+}. At wk 6, i.e., 3 wks post boost, cross sero-neutralization activities against all S_{CoV-2} variants were detectable, although at significantly lesser extents against S_{Beta}, S_{Delta} and S_{Delta+}. From wk 6 to wk 10, cross sero-neutralization against S_{Beta}, S_{Delta}, or S_{Delta+} gradually and significantly decreased.

At wk 15, groups of mRNA-1273-primed and -boosted mice received i.n. escalating doses of 1×10⁶, 1×10⁷, 1×10⁸, or 1×10⁹ TU/mouse of LV::S_{Beta-2P} (FIG. 2A). Control mRNA-1273-primed and -boosted mice received i.n. 1×10⁹ TU of an empty LV (LV Ctrl). In parallel, at this time point, mRNA-1273-primed and -boosted mice were injected i.m. with 1 μg of mRNA-1273 or PBS. Unprimed, age-matched mice received i.n. 1×10⁹ TU of LV::S_{Beta-2P} or PBS.

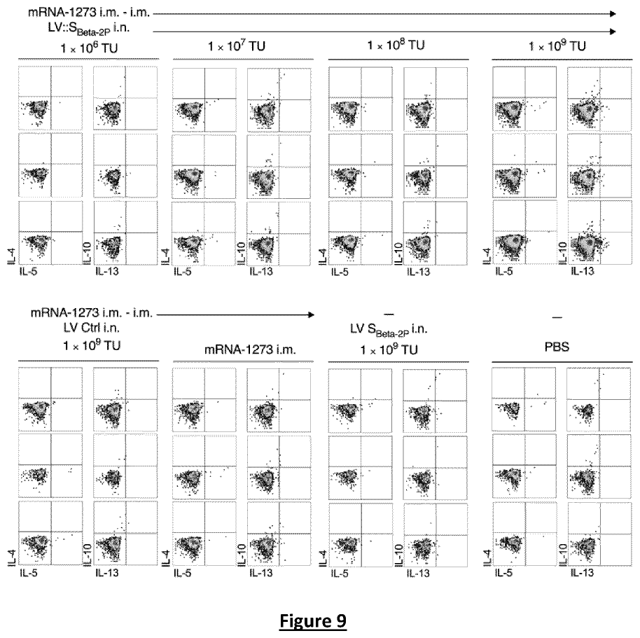

In mRNA-1273-primed mice, serum anti-S_{CoV-2} and anti-RBD IgG were detected at wk 3, increased after mRNA-1273 boost as studied at wk 6 and 10, and then decreased at wk 17 in the absence of additional boost (FIG. 8A). In the mRNA-1273-primed and -boosted mice, boosted at wk 15, a significant increase was observed in the titer of anti-S_{CoV-2} IgG in the mice injected with 1×10⁸ or 1×10⁹ TU of LV::S_{Beta-2P} or a third dose of mRNA-1273 (FIG. 2C). The titers of anti-S_{CoV-2} IgA were higher in the mice injected with 1×10⁹ TU of LV::S_{Beta-2P} than those injected with a third dose of mRNA-1273 (FIG. 2C). At the mucosal level, at this time point, titers of anti-S_{CoV-2} and anti-RBD IgG in the total lung extracts increased in a dose-dependent manner in LV::S_{Beta-2P}-injected mice, with the highest dose of LV::S_{Beta-2P} was comparable to the third i.m. dose of mRNA-1273 (FIG. 8B). Importantly, significant titers of lung anti-S_{CoV-2} IgA were only detected in LV::S_{Beta-2P}-boosted mice but were barely detectable in their counterparts boosted late via i.m. with mRNA-1273 (FIG. 8B).

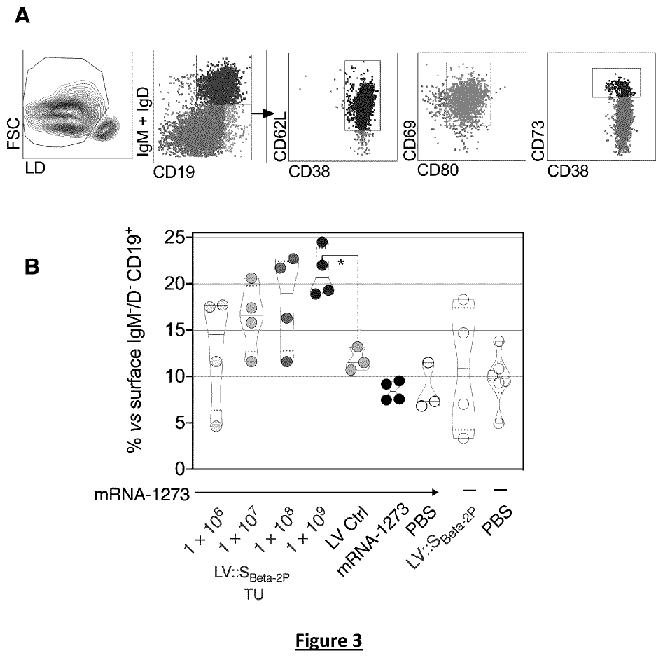

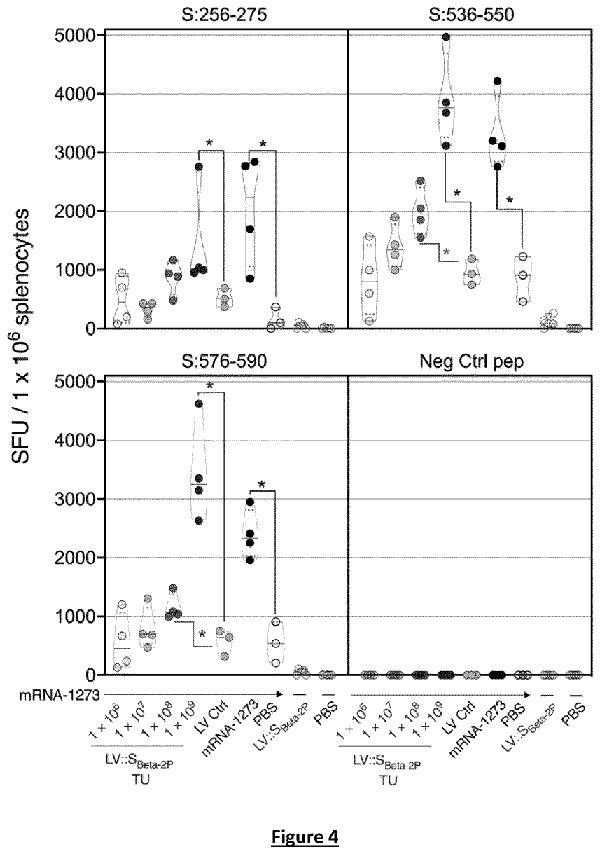

At the mucosal cellular level, inside the lung IgM⁻/IgD⁻ CD19⁺ (Ig switched) B-cell population, increased percentages of CD38⁺ CD62L⁺ CD69⁺ CD73⁺ CD80⁺ cells, which may constitute resident memory B cells, were detected in a dose-dependent manner in mice boosted i.n. with LV::S_{Beta-2P}, but not in their mRNA-1273 i.m.-boosted counterparts (FIG. 3A, B).

Features of Systemic and Mucosal T-Cell Immunity after i.n. LV::S_{Beta-2P} Boost in Previously mRNA-1273-Primed and -Boosted Mice

Systemic anti-S_{CoV-2} T-cell immunity was assessed by IFN-γ-specific ELISPOT in the spleen of individual mice immunized following the above-mentioned regimen (FIG. 2A) after in vitro stimulation with individual S:256-275, S:536-550 or S:576-590 peptide, encompassing immunodominant S_{CoV-2} regions for CD8⁺ T cells in H-2^{b} mice (Ku M W, et al. Cell Host Microbe, 29(2), 236-249 e236, 2021). Importantly, the weak anti-S CD8⁺ T-cell immunity, detectable in the spleen of mRNA-1273 primed-boosted mice at wk 17, largely increased following i.n. boosting with 1×10⁸ and 1×10⁹ TU/mouse of LV::S_{Beta-2P} (FIG. 4). The booster effect of the 1×10⁹ TU dose of LV::S_{Beta-2P} i.n. was comparable, or had a tendency to be higher than, the additional dose of mRNA-1273 i.m. on the systemic T-cell immunity.

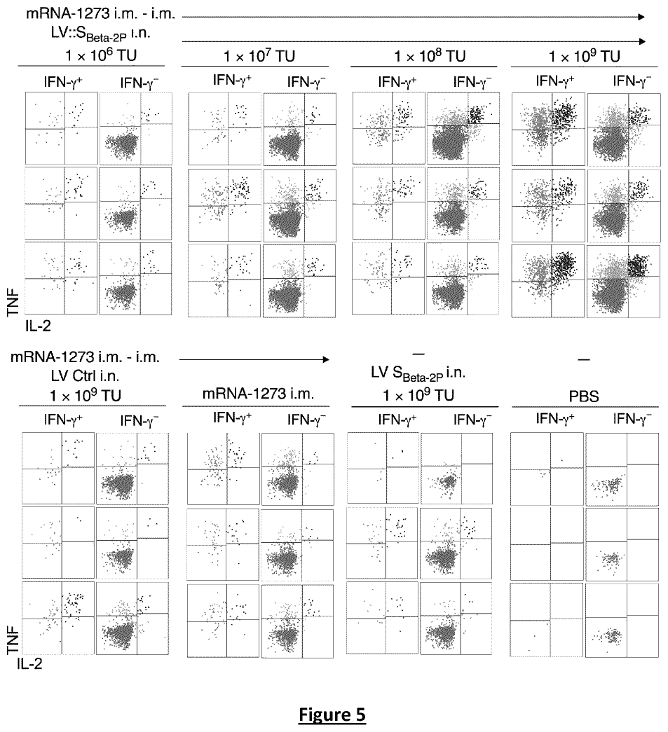

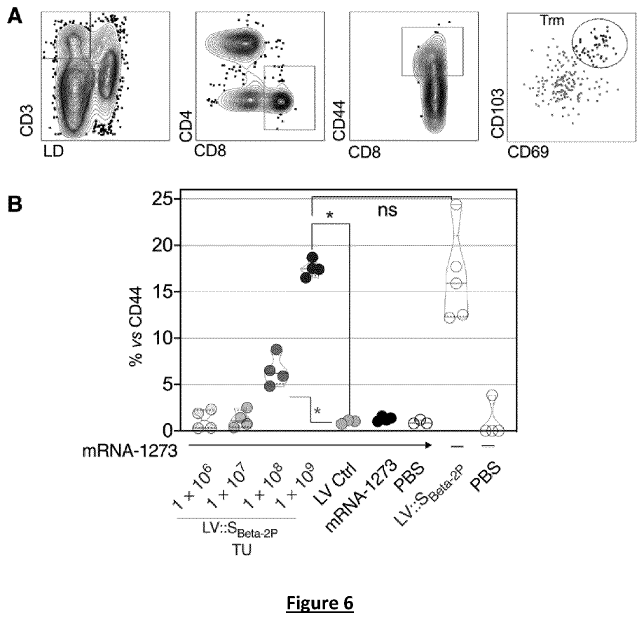

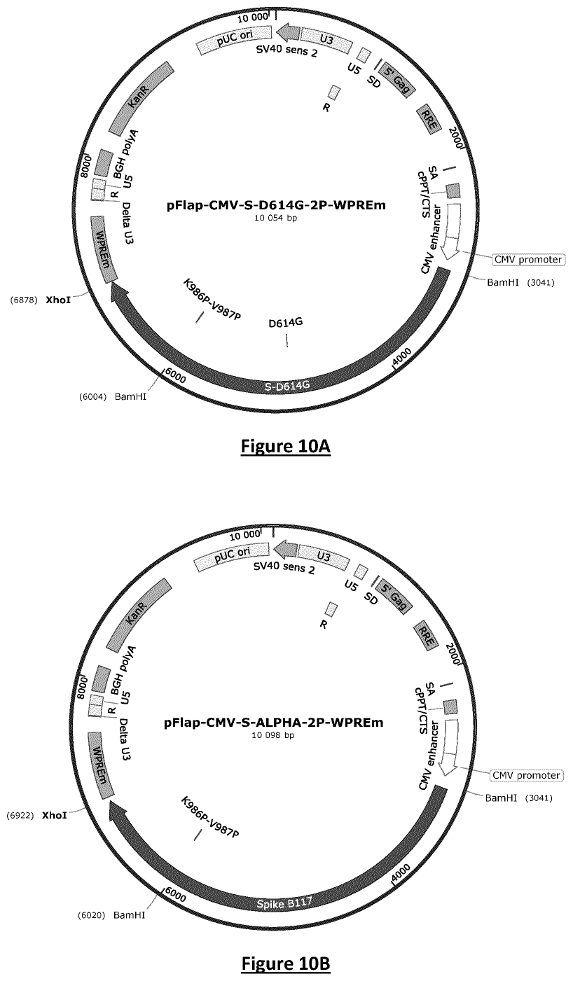

In parallel, in the same animals, the mucosal anti-S_{CoV-2} T-cell immunity was assessed by intracellular Tc1 and Tc2 cytokine staining in T-cell enriched fraction from individual mice, after in vitro stimulation with autologous bone-marrow-dendritic cells loaded with a pool of S:256-275, S:536-550 and S:576-590 peptides (FIG. 5A). In previously mRNA-1273-primed and -boosted mice, only a few S_{CoV-2}-specific IFN-γ/TNF/IL-2 CD8⁺ T-cell responses were detected in the lungs (FIG. 5A). The i.n. administration of LV::S_{Beta-2P} boosted, in a dose dependent manner, these Tc1 responses with sizable percentages of such Tc1 cells induced with the doses of 1×10⁸ or 1×10⁹ TU. The mRNA-1273 i.m. administration had a substantially lower boost effect on mucosal T cells (FIG. 5A, B). Tc2 responses (IL-4, IL-5, IL-10 and IL-13) were not detected in any experimental group (FIG. 9).

Importantly, the proportions of lung resident memory CD8⁺ T cells (Trm) were significantly increased in mice boosted i.n. with escalating doses of LV::S_{Beta-2P}, in net contrast to their counterparts which received i.m. a third dose of mRNA-1273. In fact, the latter did not have significant proportions of this Trm cell population which is very frequently correlated with protective potential in numerous infectious diseases (FIG. 6A, B).

Full Protective Potential of a Late LV::S_{Beta-2P} i.n. Boost in mRNA-1273-Primed and -Boosted Mice

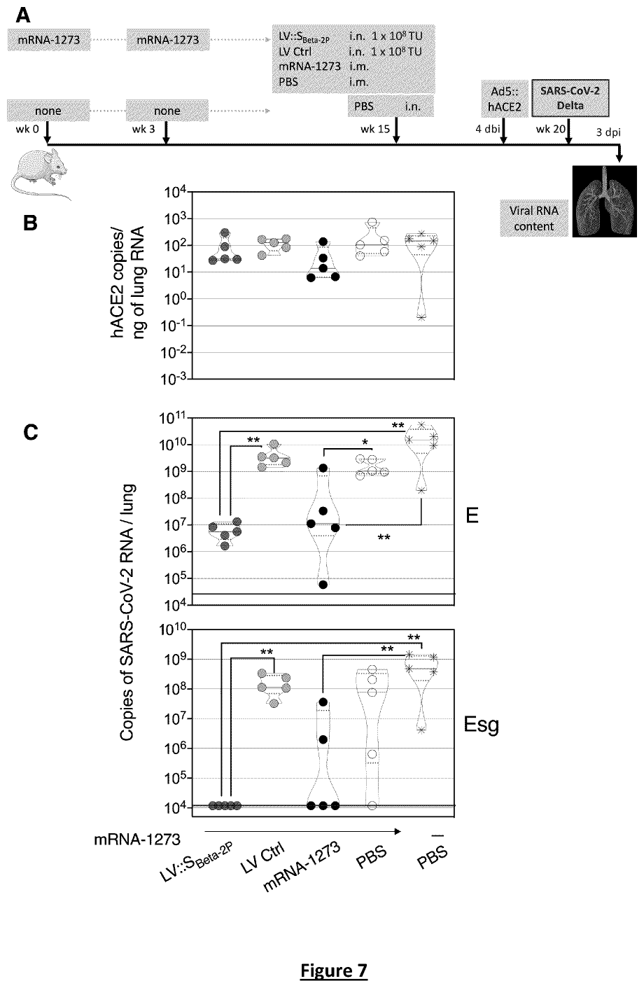

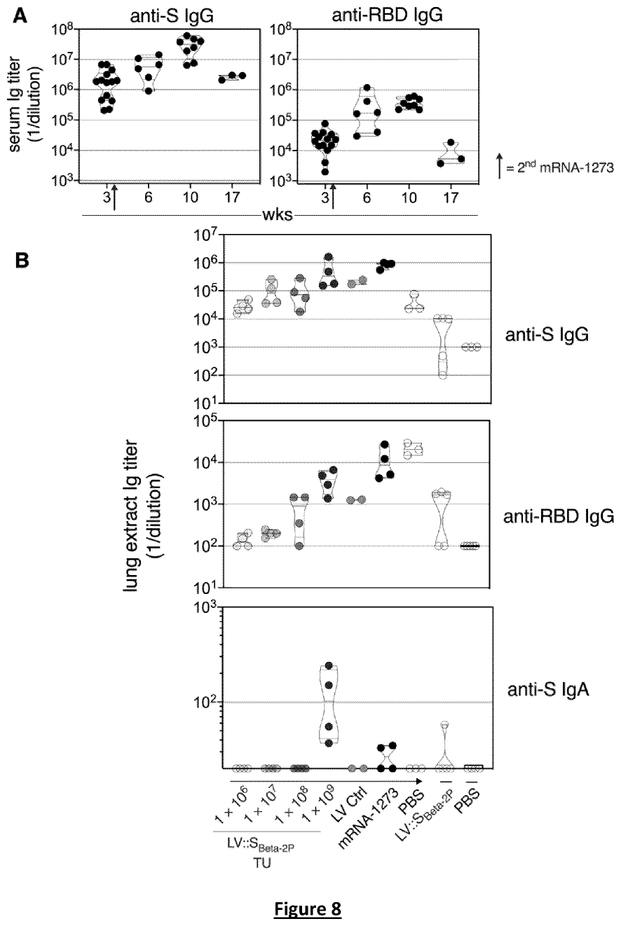

The inventors then evaluated the protective vaccine efficacy of LV::S_{Beta-2P} i.n. boost in mRNA-1273-primed and -boosted mice, following a vaccination regimen comparable to the above-mentioned one. At wk 15, mRNA-1273-primed and -boosted mice received i.n. the suboptimal dose of 1×10⁸ TU of LV::S_{Beta-2P} or control empty LV (FIG. 7A). The choice of such suboptimal dose was based on numerous previous observations from the inventors with this dose which was effective in protection in a homologous LV prime-boost experiment (Ku M W, et al. Cell Host Microbe, 29(2), 236-249 e236, 2021; Ku M W, et al. EMBO Mol Med, e14459, 2021). Control mRNA-1273-immunized mice received mRNA-1273 i.m. or PBS. Un-vaccinated, age- and sex-matched controls were left unimmunized. Four weeks after the late boost, i.e. wk 20, all mice were pre-treated with 3×10⁸ Infectious Genome Units (IGU) of an adenoviral vector serotype 5 encoding hACE2164(Ad5::hACE2) (Ku M W, et al. Cell Host Microbe, 29(2), 236-249 e236, 2021) to render their lungs permissive to SARS-CoV-2 replication (FIG. 7B). Four days later, mice were challenged with SARS-CoV-2 Delta variant, which, at the time of the present invention, i.e., November 2021, was the most expanded SARS-CoV-2 variant worldwide.

At day 3 post infection (dpi), analysis of the total lung RNA first showed that hACE-2 was expressed homogeneously in all mice after Ad5::hACE2 in vivo transduction (FIG. 7B). Lung viral loads were then determined at 3 dpi by assessing total E RNA and sub-genomic (Esg) E_{CoV-2} RNA qRT-PCR, the latter being an indicator of active viral replication (Chandrashekar A, et al. Science, 369(6505), 812-817, 2020; Tostanoski L H, et al. Nat Med, 26(11), 1694-1700, 2020; Wolfel R, Corman V M, Guggemos W et al. Virological assessment of hospitalized patients with COVID-2019. Nature, 581(7809), 465-469, 2020). In mice initially primed and boosted with mRNA-1273, and then injected i.n. with the control LV or i.m. with PBS alone, no significant protection potential was detectable against the challenge with SARS-CoV-2 Delta variant. In net contrast, the LV::S_{Beta-2P} i.n. boost drastically reduced the total E_{CoV-2} RNA content of SARS-CoV-2 and no copies of the replication-related Esg E_{CoV-2} RNA were detected in this group (FIG. 7B). The content of total E_{CoV-2} RNA was also significantly reduced in the group which received a late mRNA-1273 i.m. boost. The content of Esg E_{CoV-2} RNA was undetected in 3 out of 5 in this group.

### Full Lung Prophylaxis Against SARS-CoV-2 by One Shot or Booster Intranasal Lentiviral Vaccination in Syrian Golden Hamsters

The inventors demonstrated that a single intranasal administration of a vaccinal lentiviral vector encoding a stabilized form of the original SARS-CoV-2 Spike glycoprotein induced full lung protection of respiratory tracts and strongly reduced pulmonary inflammation in the susceptible Syrian golden hamster model against the prototype SARS-CoV-2. In addition, the inventors showed that a lentiviral vector encoding stabilized Spike of SARS-CoV-2 Beta variant (LV::S_{Beta-2P}) prevented pathology and reduced infectious viral loads in lungs and nasal turbinates following inoculation with the SARS-CoV-2 Omicron variant. Importantly, an intranasal boost with LV::S_{Beta-2P} improved cross-seroneutralization much better in LV::S_{Beta-2P}-primed hamsters than in their counterparts primed with an LV encoding Spike from the ancestral SARS-CoV-2. These results strongly suggested that an immune imprint with the original Spike sequence had a negative impact on cross-protection against new variants. The results of the inventors tackled the issue of vaccine effectiveness in people who have already been vaccinated and have vanished immunity and indicated the efficiency of LV-based intranasal vaccination, either as a single dose or as booster.

### Construction and Production of LV Expressing S Protein

Construction of LV::S_{WA1} and LV::S_{WA1-ΔF2P} were described previously (Ku M W, et al. Cell Host Microbe, 29(2), 236-249 e236, 2021; Ku M W, et al. EMBO Mol Med, e14459, 2021).

Production and titration of LV

Lentiviral particles were produced by transient calcium phosphate co-transfection of HEK293T cells with the vector plasmids pFlap/S_{Cov-2}, a vesicular stomatitis virus G Indiana envelope plasmid and an encapsidation plasmid pD64V for the production of integration-deficient vectors. Supernatants were harvested at 48 h post-transfection, clarified by 6-min centrifugation at 2500 rpm at 4° C. LV were aliquoted and stored at −80° C. Vector titers were determined by transducing HEK293T cells treated with aphidicolin. The titer, proportional to the efficacy of nuclear gene transfer, was determined as Transduction Unit (TU)/mL by qPCR on total lysates at day 3 post-transduction, by use forward and reverse primers specific to pFLAP plasmid, and forward and reverse primers specific to the host housekeeping gadph gene, as previously described (Iglesias et al., J. Gene Med., 2006, 8, 265-274).

### SDS-PAGE and Western Blotting

Six-well plates were seeded with HEK293T cells (2 10⁶ cells/well), and after overnight growth transduced with LV encoding SARS-CoV-2 S transgenes at a multiplicity of infection of 10. Cell lysates were harvested 48 h post transduction and quantified. After heating for 5 min at 95° C. bolt with Bolt sample buffer, samples were loaded on a precast Bolt 4-12% Bis-Tris gel (Invitrogen). Proteins were transferred to a nitrocellulose membrane using an iBlot2 dry blotting system (Invitrogen), and membrane was blocked with TBST blocker (Tris-buffered saline (TBS) containing 0.2% Tween 20 and 5% milk). Following 1 h blocking, the membrane was incubated overnight with an anti-SARS-CoV-2 S2 rabbit polyclonal antibody (SinoBiological 40590-T62) in TBST blocker. The membrane was then washed three times with TBST for 10 min and subsequently incubated for 1 h with 1:2,500 DyLight 800-conjugated goat anti-rabbit IgG (H+L) secondary antibody (Invitrogen, Cat #SA5-35571) in TBST Blocker. Finally, the membrane was washed three times with TBST for 10 min and developed using an ODYSSEY CLx Infrared Imaging System (Li-COR). E-PAGE SeeBlue Pre-stained Standard (Invitrogen) was used as ladder.

### Hamsters

Male Mesocricetus auratus Syrian golden hamsters (Le Genest Saint Isle, France) were purchased mature and weighed between 80 to 100 gr at the beginning of the experiments. Hamsters were housed in individually ventilated cages under specific pathogen-free conditions during the immunization period. For SARS-CoV-2 infection these hamsters were transferred into individually filtered cages placed inside isolators in the animal facility of Institut Pasteur. Prior to i.m. or i.n. injections, hamsters were sedated with isoflurane inhalation or i.p. injection of Ketamine (Imalgene, 100 mg/kg) and Xylazine (Rompun, 5 mg/kg).

### Ethical Approval of Animal Studies

Experimentation on hamsters was realized in accordance with the European and French guidelines (Directive 86/609/CEE and Decree 87-848 of 19 Oct. 1987) subsequent to approval by the Institut Pasteur Safety, Animal Care and Use Committee, protocol agreement delivered by local ethical committee (CETEA #DAP200007) and Ministry of High Education and Research (APAFIS #24627-2020031117362508 v1).

### Production of SARS-CoV-2 Spike Proteins

Codon-optimized nucleotide fragments encoding a stabilized version of SARS-CoV-2 WA1 or Omicron BA.1 spike (HexaPro) ectodomain (followed by a foldon trimerization motif) and WA1 or Omicron BA.1 RBD proteins containing C-terminal tags (His×8-tag, Strep-tag, and AviTag) were synthesized and cloned into pcDNA3.1/Zeo⁽⁺⁾ expression vector (Thermo Fisher Scientific). Recombinant proteins were produced by transient transfection of exponentially growing Freestyle 293-F suspension cells (Thermo Fisher Scientific, Waltham, MA) using polyethylenimine (PEI) precipitation method as previously described (PMID: 25910833). Proteins were purified from culture supernatants by high-performance chromatography using the Ni Sepharose® Excel Resin according to manufacturer's instructions (GE Healthcare), dialyzed against PBS using Slide-A-Lyzer® dialysis cassettes (Thermo Fisher Scientific), quantified using NanoDrop 2000 instrument (Thermo Fisher Scientific), and controlled for purity by SDS-PAGE using NuPAGE 3-8% Tris-acetate gels (Life Technologies), as previously described (PMID: 25910833).

### Humoral Response

Immunoglobulin G (IgG) Abs were detected by an enzyme-linked immunosorbent assay (ELISA) by use of recombinant stabilized S_{CoV-2} and RBD proteins from SARS-CoV-2 WA1 or Omicron strains. Nunc Polysorp ELISA plates (ThermoFisher, 475094) were coated at 1 μg/mL in 50 mM Na2CO3 pH 9.6 at 4° C. overnight. After incubation, plates were washed with 1×PBS+0.05% Tween-20 (PBST) and blocked with PBST+1% BSA for 2 to 3 h at 37° C. Plates were incubated with serial dilutions of sera in PBS-T+1% BSA for 1.5 hr at 37° C. Following washes, rabbit anti-hamster IgG-horseradish peroxidase conjugate (Jackson Immuno Research, M37470) was used as secondary Ab, and 3,5,3′5′-tetramethylbenzidine (Eurobio Scientific, 5120-0047) was used as the substrate to detect Ab responses. Reactions were stopped with 50 μL of 2 M sulfuric acid. Endpoint titers were calculated as the highest serum dilution that resulted in an absorbance value greater than that mean +3SD of pre-immune sera.

### SARS-CoV-2 Inoculation

Hamsters were anesthetized by i.p. injection of Ketamine and Xylazine mixture, transferred into a biosafety cabinet 3 and inoculated i.n. with 50 μl of viral inoculum containing 0.3×10⁵ TCID₅₀ of the WA1 (Lescure et al., Lancet Infect. Dis., 2020, 20, 697-706) or the Omicron BA.1 variant (Pango lineage BA.1, GISAID: EPI_ISL_6794907 and EPI_ISL_7413964) of SARS-CoV-2 clinical isolate (Planas et al., Nature, 2022, 602, 671-675). Animals were housed in an isolator in BioSafety Level 3 animal facilities of Institut Pasteur. The organs recovered from the infected animals were manipulated according to the approved standard procedures of these facilities.

### Pseudovirus Neutralization Assay

Nab quantification was assessed via an inhibition assay which uses HEK293T cells stably expressing human ACE2 (HEK 293T-ACE2) and non-replicative S_{CoV-2} pseudo-typed LV particles which harbor the reporter luciferase firefly gene, allowing quantitation of the host cell invasion by mimicking fusion step of native SARS-CoV-2 virus, as previously described (Sterlin et al., Sci. Transl. Med., 2021, 13, eabd2223). Serum samples or clarified lung homogenates were heat inactivated at 56° C. for 30 minutes. Serial four-fold dilutions of samples diluted in 25 μl DMEM-glutamax (Gibco, 21063-029) containing 10% heat-inactivated FCS, 100 U/mL penicillin and 100 mg/mL streptomycin and 1 mM sodium pyruvate (Gibco, 11360-070) were mixed with 1 ng of S_{CoV-2} pseudo-typed LV p24 equivalent in 25 μl for 30 min at room temperature, in U-bottom plates. Samples were then transferred into clear-flat-bottom 96-well-black-plates (Corning, CLS3603) containing 2 10⁴ HEK 293T-ACE2 cells. The plates were incubated for 72 hours at 37° C. and then assayed for luciferase expression using the ONE-Glo™ Luciferase Assay System (Promega, E6120) on an EnSpire plate reader (PerkinElmer). EC50 was reported as the reciprocal of the serum dilution conferring 50% of infection of HEK 293T-ACE2 cells by lentiviral vectors bearing the indicated S_{CoV-2} variants.

### Determination of Viral Loads in the Organs

Lungs and nasal turbinates (NT) were removed aseptically and immediately frozen at −80° C. RNA from circulating SARS-CoV-2 was prepared from lungs as recently described (Ku M W, et al. Cell Host Microbe, 29(2), 236-249 e236, 2021). Briefly, lung homogenates were prepared by thawing and homogenizing of the organs using lysing matrix A (MP Biomedicals, 116913050-CF) in 500 μl of ice-cold PBS in an MP Biomedical Fastprep 24 Tissue Homogenizer and were used to determine viral loads by E-specific qRT-PCR. Alternatively, total RNA was prepared from lungs or NT by addition of lysing matrix D (MP Biomedical, 116910050-CF) containing 1 mL of TRIzol reagent (ThermoFisher, 15596026) and homogenization at 30 s at 6.0 m/s twice using MP Biomedical Fastprep 24 Tissue Homogenizer. These RNA preparations were used to determine viral loads by Esg-specific qRT-PCR or inflammatory mediators.

SARS-CoV-2 E gene or E sub-genomic mRNA (Esg RNA), was quantitated following reverse transcription and real-time quantitative TaqMan® PCR, using SuperScript™ III Platinum™ One-Step qRT-PCR Kit (Invitrogen, 11732020) and specific primers and probe (Eurofins) as previously described (Corman et al. Euro Surveill. 2020, 25(3); Wolfel et al., Nature 2020, 581(7809):465-9). The standard curve of Esg mRNA assay was performed using in vitro transcribed RNA derived from PCR fragment of “T7 SARS-CoV-2 Esg mRNA”. The in vitro transcribed RNA was synthesized using T7 RiboMAX Express Large Scale RNA production system (Promega, P1320) and purified by phenol/chloroform extraction and two successive precipitations with isopropanol and ethanol. Concentration of RNA was determined by optical density measurement, diluted to 10⁹ genome equivalents/μL in RNAse-free water containing 100 μg/mL tRNA carrier, and stored at −80° C. Serial dilutions of this in vitro transcribed RNA were prepared in RNAse-free water containing 10 μg/ml tRNA carrier to build a standard curve for each assay. PCR conditions were: (i) reverse transcription at 55° C. for 10 min, (ii) enzyme inactivation at 95° C. for 3 min, and (iii) 45 cycles of denaturation/amplification at 95° C. for 15 s, 58° C. for 30 s. PCR products were analyzed on an ABI 7500 Fast real-time PCR system (Applied Biosystems). RNA copy values were extrapolated from the standard curve and multiplied by the volume to obtain RNA copies per organ. The limit of detection was based on the standard curve and defined as the quantity of RNA that would give a Ct value of 40.

The qRT-PCR quantification of inflammatory mediators in the lungs and brain of hamsters was performed in total RNA extracted by TRIzol reagent, as recently detailed (Ku M W, et al. Cell Host Microbe, 29(2), 236-249 e236, 2021).

### Histopathology

Samples from the lungs of hamsters were fixed in formalin for 7 days and embedded in paraffin. Paraffin sections (5-μm thick) were stained with Hematoxylin and Eosin (H&E). Histopathological lesions were qualitatively described and when possible scored, using: (i) distribution qualifiers (i.e., focal, multifocal, locally extensive or diffuse), and (ii) a five-scale severity grade, i.e., 1: minimal, 2: mild, 3: moderate, 4: marked and 5: severe. In some cases, serial sections were prepared for immunohistochemistry (IHC) analyses. IHC was performed as previously described (Ku M W, et al. EMBO Mol Med, e14459, 2021). Rabbit anti-N_{CoV-2} antibody (Novus Biologicals, NB100-56576) and biotinylated goat anti-rabbit Ig secondary antibody (Dako, E0432) were used in IHC. Slides were scanned using the AxioScan Z1 (Zeiss) system and images were analyzed with the Zen 2.6 software.

### Statistical Analysis

Statistical significance was assigned when P values were <0.05. ELISA titers were log₁₀ transformed prior to statistical analysis. For comparison of two groups, nonparametric Mann-Whitney test was used. To compare more than 2 experimental groups, Kruskal-Wallis ANOVA with Dunn's multiple comparisons tests were applied. Differences in neutralizing activity of VoCs were analyzed by two-way ANOVA with Sidak test for multiple comparisons. Tests were performed using GraphPad Prism software (Version 9, Graphpad Software, La Jolla, CA, United States).

### Results

### Immunogenicity of LV Encoding Various S_{CoV-2} Forms

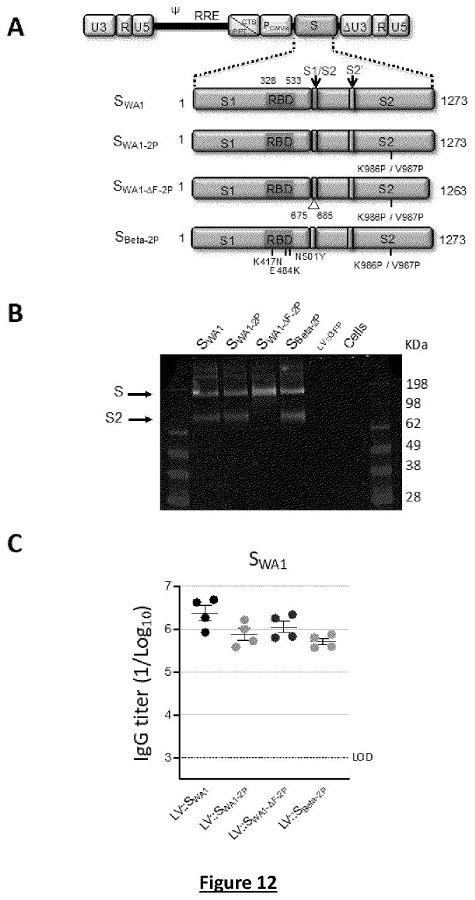

Non-integrative LV encoding stabilized conformers of S_{CoV-2} under transcriptional control of the cytomegalovirus (CMV) immediate early promoter (P_{CMVie}) were constructed (FIG. 12A). The first two S_{CoV-2} conformers were derived from a human codon-optimized full-length membrane anchored ancestral WA1 S_{CoV-2} (Ku M W, et al. Cell Host Microbe, 29(2), 236-249 e236, 2021). LV::S_{WA1-2P} encodes a S_{WA1} which harbors two stabilizing K⁹⁸⁶P and V⁹⁸⁷P substitutions in the hinge loop of the S2 domain. LV::S_{WA1ΔF-2P} encodes a S_{WA1} which, in addition to the two K⁹⁸⁶P and V⁹⁸⁷P substitutions, is deleted of the loop encompassing the S1/S2 furin cleavage site (675-QTQTNSPRRAR-685 of SEQ ID NO: 27) for further stability at the prefusion state (McCallum et al., Nat. Struct. Mol. Biol., 2020, 27, 942-949; Launay et al., EBioMedicine 2022, 75, 103810). S_{Beta-2P} is from the Beta (B.1.351) VoC and contains the two K⁹⁸⁶P and V⁹⁸⁷P substitutions. S_{Beta} differs from S_{WA1}, notably by the N⁵⁰¹Y/K⁴¹⁷N/E⁴⁸⁴K mutations located in the RBD (Tegally et al., Nature 2021, 592, 438-443). Whereas pseudoviruses carrying S_{WA1} were neutralized by sera from individuals vaccinated with the currently approved vaccines, those presenting these RBD mutations moderately-to-strongly resist neutralization (Kuzmina et al., iScience 2021, 24, 103467). This observation provided a rational for adapting the S sequence variant for further vaccination. Expression of S_{CoV-2} immunogens in HEK293T cells transduced with the four LV was confirmed by Western blot on total cell lysates (FIG. 12B). As expected, the S2 furin cleavage product was only detected in the cells transduced by LV encoding S_{WA1}, S_{WA1-2P} or S_{Beta-2P} which harbor an intact furin cleavage site.

To compare the immunogenicity of LV::SWA1, LV::S_{WA1-2P}, LV::S_{WA1ΔF-2P} and S_{Beta-2P}, hamsters (n=4/group) were immunized by a single i.m. injection of 1×10⁸ TU of either LV. Five weeks (wks) later, high serum titers of anti-SWA1 IgG antibodies were induced by all LV studied (FIG. 12C). As no significant difference in immunogenicity between these LV was observed, LV::S_{WA1ΔF-2P}, hereafter referred to as “LV::S”, was selected for evaluation of protection against homologous SARS-CoV-2.

Induction of Robust Humoral Responses Against SARS-CoV-2 by a Single i.n. LV::S Administration

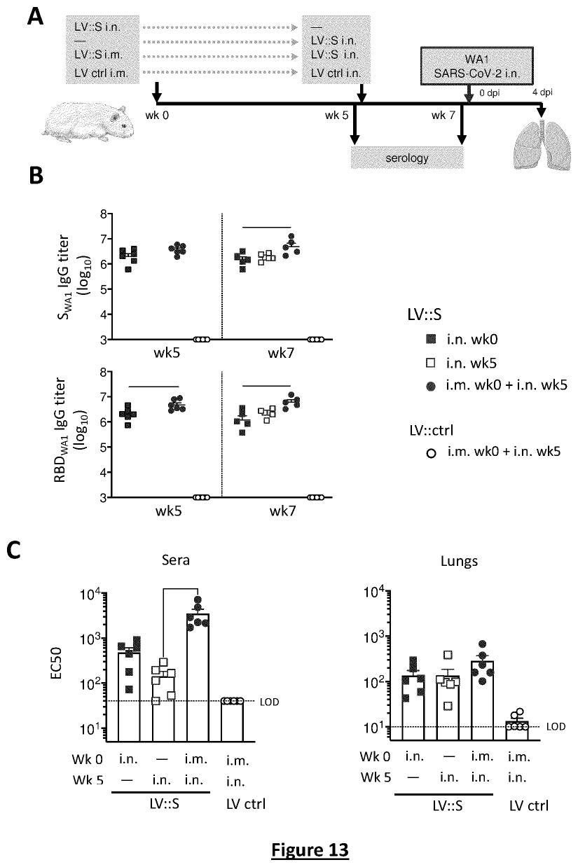

The inventors recently showed that LV::S used in a prime (i.m.)-boost (i.n.) protocol significantly improved protection against SARS-CoV-2 compared to a single i.m. injection in the hamster model (Ku M W, et al. Cell Host Microbe, 29(2), 236-249 e236, 2021). Here, the inventors evaluated the protective potential of a single i.n. administration of LV::S against the ancestral WA1 SARS-CoV-2. Hamsters (n=6/group) were immunized i.n. with a single injection of 1×10⁸ TU of LV::S at wk 0 or at wk 5 (FIG. 13A). As a positive control, a group of hamsters was primed i.m. with 1×10⁸ TU of LV::S at wk 0 and then boosted i.n. at wk 5 with the same amount of LV::S. Control hamsters received, following the same regimen, equivalent amounts of an LV expressing a green fluorescent protein, as an irrelevant antigen (LV ctrl). At wk 7, all animals were challenged i.n. with 0.3×10⁵ TCID₅₀ of WA1 SARS-CoV-2 (FIG. 13A). Before the challenge, pre-immune sera and those from the LV ctrl group were tested negative for anti-S_{WA1} and -RBD_{WA1} antibodies (FIG. 13B). Following a single LV::S i.n. injection, all animals mounted high titers of anti-S_{WA1} and -RBD_{WA1} IgG. These antibody titers are obtained as soon as 2 wks post-immunization as shown by hamsters vaccinated at wk 5. The serum IgG titers remained stable until wk 7. At wk 7, significantly lower anti-SWA1 and anti-RBD_{WA1} IgG titers were detected in the groups injected i.n., compared to the i.m.-i.n. group. The sero-neutralization activity was evaluated by use of pseudoviruses harboring S_{WA1}. In agreement with the anti-RBD IgG titers, sero-neutralizing activities were lower in the hamsters immunized with a single i.n. injection, compared to the i.m.-i.n. group (FIG. 13C). Despite comparable anti-S and anti-RBD IgG titers at 2 or 7 wks after i.n. injection, sera from hamsters vaccinated at the earlier time point exhibited a slightly higher neutralizing capacity, suggesting the requirement of an antibody maturation over time to reach efficient neutralizing potential. However, all vaccinated groups had equivalent neutralizing capacities in their total lung homogenates, four days post SARS-CoV-2 inoculation (4 dpi) (FIG. 13C). The virus neutralizing activity in lungs can be a more relevant correlate of protection than that detectable in sera.

Protection Against Homologous SARS-CoV-2 Challenge Induced by a Single i.n. LV::S Administration

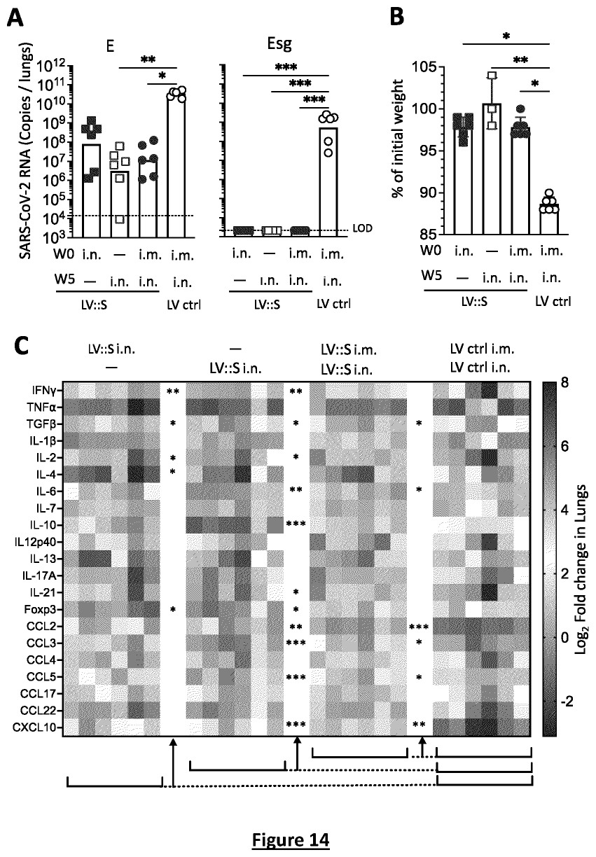

In the lungs of LV::S-vaccinated individuals of either i.m.-i.n. or single i.n. groups, ˜2-to-4 log₁₀ decreases of viral contents were observed compared to the LV ctrl group, as determined by qRT-PCR detecting the SARS-CoV-2 Envelop (E) RNA at 4 dpi (FIG. 14A, left panel). Lung viral content measured by a sub-genomic E RNA (Esg) qRT-PCR is an indicator of active viral replication (Wolfel et al., Nature 2020, 581(7809):465-9). This analysis showed a complete absence of replicating virus in the three vaccinated groups versus a geometric mean±SD of (5.4±6.8)×10⁸ copies of Esg RNA of SARS-CoV-2/lungs in the LV ctrl group (FIG. 14A, right panel). At 4 dpi, in accordance with the protection observed, only 2-3% weight loss was detected in the hamsters vaccinated, either by i.n. alone or by i.m.-i.n. prime-boost regimen, compared to 12% weight loss in the hamsters which received LV ctrl (FIG. 14B). As evaluated by qRT-PCR at 4 dpi in the total lung homogenates of the LV::S-vaccinated and SARS-CoV-2 challenged hamsters, marked decreases were detected in the expression of inflammatory IFN-γ, TGF-α, IL-6 cytokines, anti-inflammatory IL-10 cytokines, and CCL2, CCL3, CCL5 and CXCL10 chemokines, as well as FoxP3, in comparison to their LV ctrl-injected and challenged counterparts (FIG. 14C). Changes in inflammatory markers was particularly noticeable for the group receiving the i.n. administration 2 weeks before the challenge. In agreement with these results, a positive correlation was found between viral loads and inflammation (r=0.46, p<0.05), whereas the weights were inversely correlated with viral loads and inflammation (r=−0.6842, p<0.001 and r=−0.56, p<0.01, Spearman test), respectively.

Reduced Infection-Driven Inflammation in Hamsters Vaccinated with a Single i.n. LV::S Administration

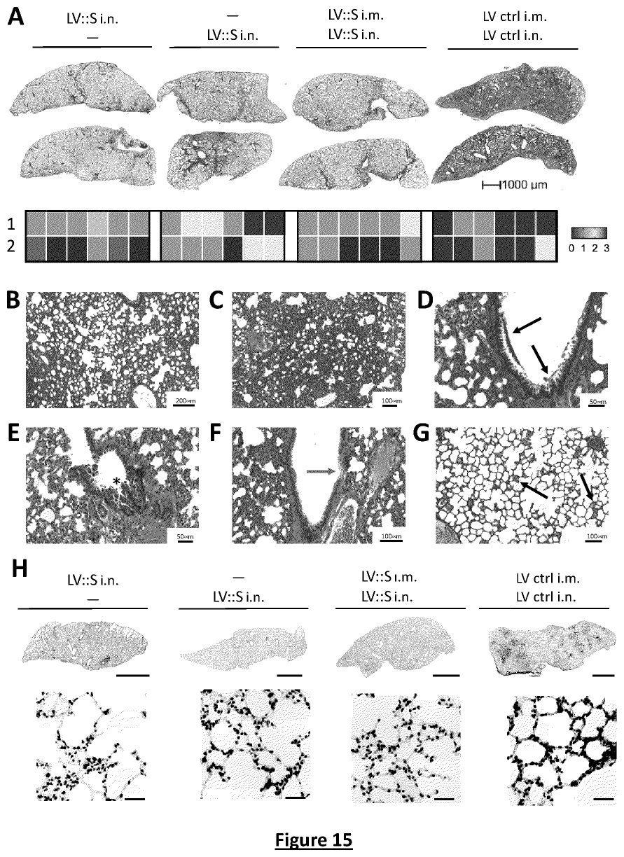

On lung histopathological examination, vaccinated controls demonstrated lung infiltration (FIG. 15A) and severe alveolo-interstitial inflammation (FIG. 15B) leading to dense pre-consolidation areas (FIG. 15C). These lungs also displayed bronchiolar lesions, with images of epithelial sloughing of individual or clustered cells (FIG. 15D) and of hyperplastic epithelial growth producing papillary projections (FIG. 15E) or intraluminal epithelial folds (FIG. 15F). In vaccinated groups the interstitial (FIG. 15A) and alveolar (FIG. 15G) lesions were minimal to moderate. Immunohistochemistry analysis of the lungs of LV ctrl-treated and infected hamsters, with a SARS-CoV-2 nucleocapsid protein (N_{CoV-2})-specific polyclonal antibody, detected numerous clusters of N_{CoV-2}⁺ cells in the bronchial epithelial cells (not shown) and in the interstitium (FIG. 15H, right panels). In contrast, the severity of inflammation was reduced in LV::S-vaccinated animals. When detectable, the inflamed zones still contained N_{CoV-2}⁺ cells, indicating that, although viral replication has been controlled (FIG. 14A), infiltration and virus remnants have not yet been fully resorbed at the early 4 dpi time point.

These data collectively indicated that immunization with a single i.n. administration of LV::S was as efficient as an i.m. prime followed by a i.n. boost regimen and conferred strong protective immunity against an homologous SARS-CoV-2 infection.

LV::S_{Beta-2P} prime (i.m.)-boost (i.n.) vaccination cross-protects against Omicron variant

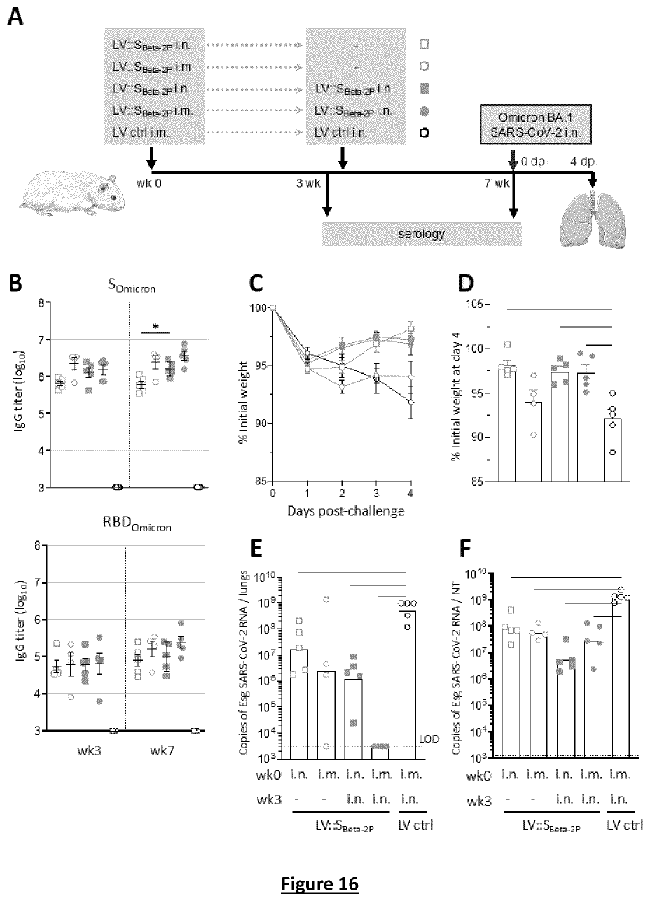

Given the dynamics of the pandemic, an important question is the ability of vaccines to induce cross-protection against new VoCs. Based on a series of LVs encoding for S from various VoCs, the inventors recently selected LV::S_{Beta-2P} as the best candidate to generate the broadest spectrum of cross-neutralization potential (Vesin, B., et al. Mol Ther 30, 2984-2997, 2022). To evaluate the efficacy of LV::S_{Beta-2P} in the hamster model against SARS-CoV-2 Omicron, hamsters (n=4-5/group) were primed i.m. or i.n. at wk0 with 1×10⁸ TU of LV::S_{Beta-2P}. At wk3, one group of each were boosted i.n. with the same dose of LV::S_{Beta-2P} (FIG. 16A). All groups were challenged at wk7 with 0.3×10⁵ TCID₅₀ of SARS-CoV-2 BA.1 Omicron sub-variant (Planas et al., Nature 2022, 602(7898):671:5). S_{Omicron} harbors 32 mutations compared to S_{WA1}. Among these mutations, 15 are located in the RBD. Infection of hamsters with this BA.1 Omicron strain, isolated from a patient, resulted in a significant decrease in weight (FIG. 16B).

Robust cross-reactive serum IgG titers were detected by ELISA against S_{Omicron} and RBD_{Omicron} in all LV::S_{Beta-2P}-vaccinated hamsters (FIG. 16B). No significant differences in antibody titers between the groups were observed 3 wks post-prime. Antibody levels remained stable after the single injections, while a significant increase in anti-S_{Omicron} titers was observed in the animals primed and boosted i.n. By contrast, anti-RBD antibody titers continued to rise in all vaccinated groups over time (FIG. 16B, lower panel).

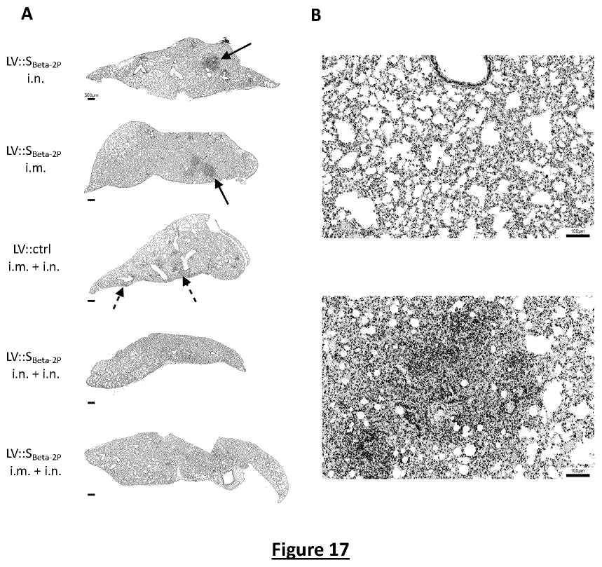

Following challenge, hamsters vaccinated by a single i.m. injection of LV::S_{Beta-2P} or those who received LV ctrl gradually lost weight (FIG. 16C). Hamsters vaccinated i.n. with LV::S_{Beta-2P} exhibited less than 5% of weight loss, without signs of morbidity (FIG. 16D). At 4 dpi, viral contents in the lungs and in nasal turbinates analyzed. High viral contents were detected in both organs of the LV ctrl-injected group (FIGS. 16E, 16F). In contrast, no Esg RNA was detected from the lungs of the i.m.-i.n. group and significant reductions of ˜2 log were observed in the other groups (FIG. 16E). Of note, the i.m. vaccinated hamster which did not control viral replication had the highest weight loss. Although also significantly reduced, active viral replication was still detectable in the NT of all hamsters, indicating that LV-based i.n. vaccination, despite its strong efficacy in the protection of the lungs, does not fully prevent nasal infection (FIG. 17F). However, an i.n. boost, regardless of the route of prime led to a better efficacy over a single vaccine administration in the control and the spread of infection in the respiratory tract tissues.

### Decrease of Virus Content as Determined by Immunohistochemistry in LV:: S_{Beta-2P}-Vaccinated Hamsters.

At 4 dpi, histopathological analysis of the lung sections in the ctrl group showed similar lesions detailed in the FIG. 15H (FIG. 17). Immunohistochemistry images displayed a generally less abundant N_{CoV-2} staining in mice boosted i.n or i.m, relative to the primed-only and LV ctrl-injected animals, although there was a relatively high degree of intra-group variation (FIG. 17). In addition, the inventors did not observe a tight correlation between the extent of the IHC signal and the Esg qRT-PCR quantifications, indicating that part of the immunostained antigen corresponds to non-replicating virus remnants.

Altogether, these results showed that while a single i.n. immunization with LV can be enough to control the infection, an LV-based i.n. administration will be better adapted to boost a previously induced anti-COVID-19 immunity.

Induction of Cross-Reactive Antibodies in LV::S_{WA1-2P}-Primed and LV::S_{Beta-2P}-Boosted Hamsters

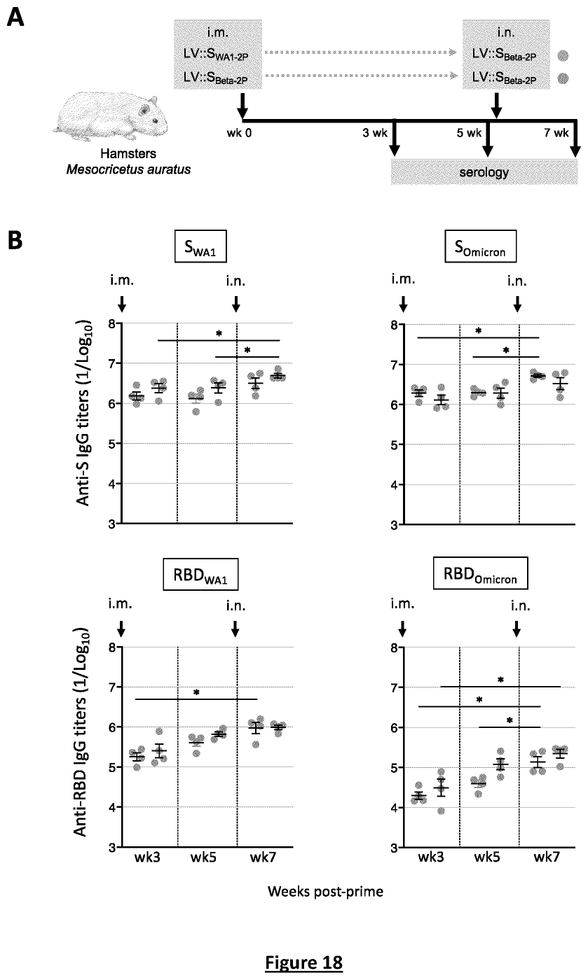

The inventors then evaluated the efficacy of an LV::S_{Beta-2P} i.n. boost in animals previously exposed to S_{WA1}. Hamsters (n=4/group) were primed i.m. at wk0 with 1×10⁸ TU of LV::S_{WA1-2P} or LV::S_{Beta-2P}. At wk5, both groups were boosted i.n. with 1×10⁸ TU of LV::S_{Beta-2P} (FIG. 18A). Robust serum IgG titers were detected against S and RBD proteins at any post-prime time point tested, in all vaccinated hamsters (FIG. 18B). After the prime or after the boost, comparable kinetic profiles and intensities of S_{WA1}- or S_{Omicron} specific antibody responses were observed (FIG. 18B, upper panels). Either the homologous or the heterologous i.n. boost marginally increased the anti-S antibody titers by 1.8- or 2.5-fold, respectively. By contrast, ˜4- to 10-fold lower serum IgG responses against RBD_{Omicron} were measured compared to RBD_{WA1} (FIG. 18B, lower panels). However, anti-RBD_{Omicron} IgG titers were significantly better improved by heterologous boost than by homologous boost with a 3.8- versus 1.7-fold increase, respectively.

Anti-S_{CoV-2} Antibody Imprinting in LV::S-Primed and LV::S_{Beta-2P}-Boosted Hamsters

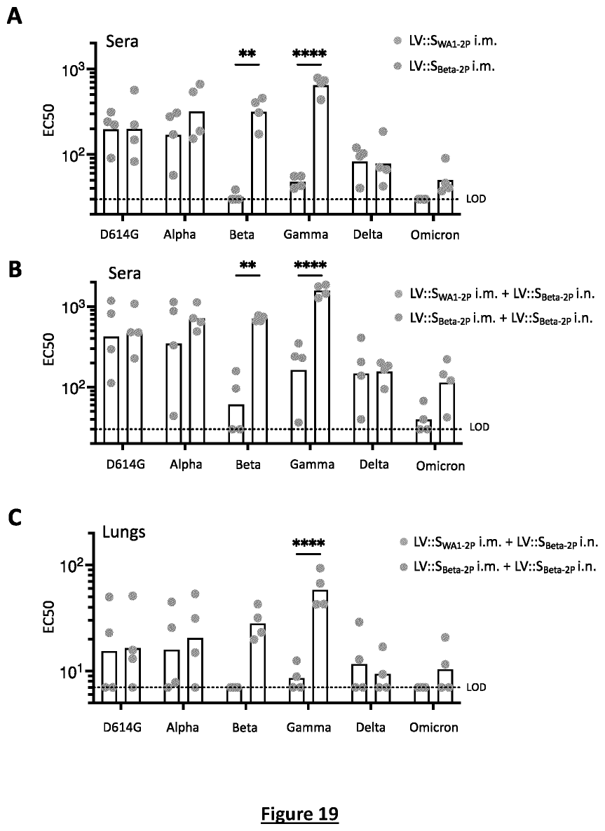

Five weeks post i.m. injection, both LV::S_{WA1-2P} and LV::S_{Beta-2P} induced high sero-neutralizing activities against pseudoviruses harboring S_{D614G} or S_{Alpha} (FIG. 19A). Cross-neutralizing activity against S_{D614G}, S_{Alpha}, and S_{Delta} was similar in the two groups of immunized hamsters. Of note, after a single i.m. injection, only LV::S_{Beta-2P}-immunized hamsters exhibited sero-neutralization activity against all S variants, although weaker against S_{Omicron}.

LV::S_{Beta-2P} i.n. boost increased the cross sero-neutralization potential against all VoCs in both groups (FIG. 19B). Although the levels of neutralizing antibodies were improved in the sera from the LV::S_{WA1-2P}-primed and LV::S_{Beta-2P}-boosted hamsters, they were barely able to cross-neutralize pseudoviruses harboring S_{Beta} and totally unable to cross-neutralize pseudoviruses harboring S_{Omicron} (FIG. 19B). Lung homogenates exhibited a similar profile with no cross-neutralizing activities against S_{Beta} or S_{Omicron} following the heterologous prime-boost (FIG. 19C). In net contrast, sera and lung homogenates from LV::S_{Beta-2P}-primed and LV::S_{Beta-2P}-boosted hamsters were much better able to cross-neutralize pseudoviruses harboring S_{Beta} or S_{Gamma} and—to a lesser extent—those harboring S_{Omicron}. However, this prime-boost regimen was sufficient to confer protection against SARS-CoV-2_{Omicron} challenge, as observed above (FIG. 16). Therefore, an LV::S_{Beta-2P} boost improves cross sero-neutralization much better in LV::S_{Beta-2P}-primed hamsters than in their LV::S_{WA1}-primed counterparts. These results showed a clear imprinting effect in anti-S humoral immunity, and particularly obvious against the Omicron and Beta variants.

### Discussion

LV-based platform has emerged recently as a powerful vaccination approach against COVID-19. The inventors notably demonstrated its strong prophylactic capacity at inducing protection in the lungs against SARS-CoV-2 infection when used as a systemic prime followed by mucosal i.n. boost (Ku M W, et al. Cell Host Microbe, 29(2), 236-249 e236, 2021). In the present study, as a further step toward a clinical trial, the inventors used LV encoding stabilized forms of S_{WA1} or S_{Beta}. This choice was based on data indicating that stabilization of viral envelop glycoproteins in their prefusion forms improves the yield of their production as recombinant proteins in industrial manufacturing of subunit vaccines. Moreover, it also increases the efficacy of nucleic acid-based vaccines, by raising availability of the antigen under its optimal immunogenic shape (Hsieh et al., Science, 2020, 369(6510):1501-5).

In the first part of this report, the inventors demonstrated that a single i.n. administration of an LV encoding S_{WA1} confers, as efficiently as an i.m.-i.n. prime-boost regimen, full protection of lungs in the highly susceptible hamster model, as evaluated by virological, immunological and histopathological parameters. The hamster ACE2 ortholog protein interacted efficaciously with S_{CoV-2}, readily allowing host cell invasion by SARS-CoV-2 with high replication rates. With rapid weight loss and development of severe lung pathology subsequent to SARS-CoV-2 inoculation, outbred hamsters provided a sensitive model to evaluate the efficacy of drug or vaccine candidates (Sia et al., Nature 2020, 583(7818):834-8). Hamsters represented a more challenging model than Rhesus macaques which developed only a mild COVID-19 pathology. The strong protection of the lung conferred by a single i.n. administration against homologous challenge in the hamster model was therefore an asset of primary importance. This protection most likely resulted from the development of a mucosal immunity. Induction of antigen-specific secretory dimeric IgA that could block the interaction of the virus at the mucosal level had been shown to reduce the viral shedding and to correlate with protection (Halfmann et al., Cell Rep. 2022; 39(3):110688; Munoz-Fontela et al., Nature 2020, 586(7830):509-15; Wang et al. Sci Transl Med. 2021, 13(577)). Although infectious virus was still detected in the nasal turbinates of i.n.-immunized hamsters, the significant reduction in infectious viral titers could lead to reduced transmission and dissemination as recently described by Langel S N et al. providing a means of disease control (Langel et al., Sci Transl. Med. 2022, 14(658)). Indeed, the inventors have previously shown in the mouse model that anti-S IgG and secretory IgA antibodies were generated in lungs, together with lung resident memory B and T cells following i.n. LV administration. The presence of IgA induced by LV-based SARS-CoV-2 vaccines correlated with complete pulmonary protection against the virus (Vesin, B., et al. Mol Ther 30, 2984-2997, 2022). Unfortunately, the mucosal immunity could not be assessed in the hamster model because of the lack of immunological tools, including anti-IgA antibodies and antibodies to activation/memory T-cell markers. Meanwhile, there was growing evidence that i.n. immunization provided a better protection, not only against SARS-CoV-2 ancestral strain, but also against newly emerged VoCs (Afkhami et al., Cell 2022, 185(5):896-915; Bricker et al., Cell Rep. 2021, 36(3):109400). The studies exploring this domain so far used chimpanzee adenoviral vectored vaccines that are known to be pro-inflammatory, and thus risky for use in mucosal vaccination (Coughlan et al., Mol. Ther. 2022, Adenovirus-based vaccines—a platform for pandemic preparedness against emerging viral pathogens). In net contrast, LVs are non-cytopathic and very weakly inflammatory (Ku et al. Vaccines 2021:1-16, 1988854) and much more suitable for mucosal vaccination. The fact that a single i.n. LV-based vaccine administration, either 2 or 7 wks before homologous SARS-CoV-2 challenge, elicits protection is valuable in setting clinical trials with LV-based vaccines. This platform can provide remarkable advantages for mass vaccination, with the major advantage of mucosal immunization in the reduction of viral transmission.

The continued emergence of SARS-CoV-2 VoCs prompted the inventors to expand their study by assessing the protective potential of a heterologous antigen booster which could, in terms of anti-S antibody response, mimic some aspects of a previous infection or earlier vaccination with the first-generation vaccines, mainly based on S_{WA1}. Numerous breakthrough SARS-CoV-2 infections have been observed in vaccinated individuals, showing the incomplete cross-efficacy of these vaccines (Abu-Raddad L J, et al. N Engl J Med. 2021; 385(2):187-9; Kuhlmann C, et al. Lancet. 2022; 399(10325):625-6). Recently, it has been reported that mucosal booster vaccination is needed to establish robust sterilizing immunity in the respiratory tract against SARS-CoV-2 (Tang J, et al. Respiratory mucosal immunity against SARS-CoV-2 following mRNA vaccination. Sci Immunol. 2022:eadd4853). In LV-immunized hamsters, the inventors did not detect striking differences between the ability of S_{WA1} and S_{Beta-2P} antigens to induce cross-reactive serum IgG responses against S_{CoV-2}. However, a clear distinction should be made between the protective capacity of vaccines and their ability to induce neutralizing antibodies, since T-cell responses are also major effector players against SARS-CoV-2 infection. In particular, the effectiveness of LV-based protection is not only dependent on the capacity to induce neutralizing antibody responses but also, and to a large extent, on their T-cell immunogenicity. It is noteworthy that an almost complete protection of lungs is achieved in μMT KO mice that are totally devoid of mature B-cell compartment and antibody response (Ku M W, et al. EMBO Mol Med. 2021:e14459). In addition, mucosal resident memory T cells, as well as IFNγ⁺ IL-2⁺ TNF⁺ triple positive CD8⁺ T cell effectors, are readily detectable in the lung of LV::S-primed (i.m.) and boosted (i.n.) mice [26]. Furthermore, findings obtained following natural infection largely suggest that specific T-cell immunity, which is generally less affected by mutations occurring in the S antigen of emerging SARS-CoV-2 variants, are largely effective against viral replication (Altmann D M, et al. Cell Rep Med. 2021; 2(5):100286; Mazzoni A, et al. Front Immunol. 2022; 13:801431). T-cell mediated protection is also certainly operating in hamsters. However, as mentioned above, the lack of immunological tools prevented the characterization of T-cell responses in the present study.

In the LV::S-primed and LV::S_{Beta-2P}-boosted hamsters, despite the enhanced sero-neutralizing potential against D614G, Alpha and Delta variants, largely statistically reduced cross-neutralization activities were observed against Beta and Gamma variants and no cross-neutralization activity was observed against the Omicron variant. Likewise, in the cases of influenza A viruses, a first exposure to a serotype can affect future responses to its variants (Gostic K M, et al. Science. 2016; 354(6313):722-6). This raises concerns about immune imprinting effects of previous infections or vaccinations on antibody responses, which will need to be considered when designing vaccines (Roltgen K, et al. Immune imprinting, breadth of variant recognition, and germinal center response in human SARS-CoV-2 infection and vaccination. Cell. 2022). The present study indicates that pre-exposure of the immune system to an early S variant has a negative impact on the neutralizing antibody response, measured after a late booster with a heterologous S variant. The results of the inventors corroborate recent data showing that healthcare workers infected either by SARS-CoV-2 ancestral or Alpha variant exhibit a reduced neutralizing immunity against Omicron (Reynolds C J, et al. Science. 2022; 377(6603):eabq1841). Moreover, using mRNA vaccines, Kalnin et al. also showed that heterologous boosting provided inferior neutralizing antibody titers compared to homologous boosting (Kalnin K V, et al. Vaccine. 2022; 40(9):1289-98). The hypothesis can be put forward that additional injections of the variant S sequence could be required to counteract this negative effect and to reach sufficient levels of cross-neutralization against VoCs.

Collectively, the results of the inventors demonstrated the ability of the LV as an effective vaccine delivery platform. LV was an effective and promising strategy to elicit a strong protective immunity against SARS-CoV-2 VoCs and possessed the advantage to be non-inflammatory and thus suitable for use in mucosal i.n. vaccination. The inventors have recently demonstrated the safety of LV::S_{Beta-2P} i.n. administration in mice in which the high dose of 1×10⁹ TU of LV had been injected (Vesin, B., et al. Mol Ther 30, 2984-2997, 2022). No adverse effects had been detected by lung histopathological analyses.

### Full Cross-Protective Capacity of LV::S_{Beta-2P} Against SARS-CoV-2 Omicron Variant

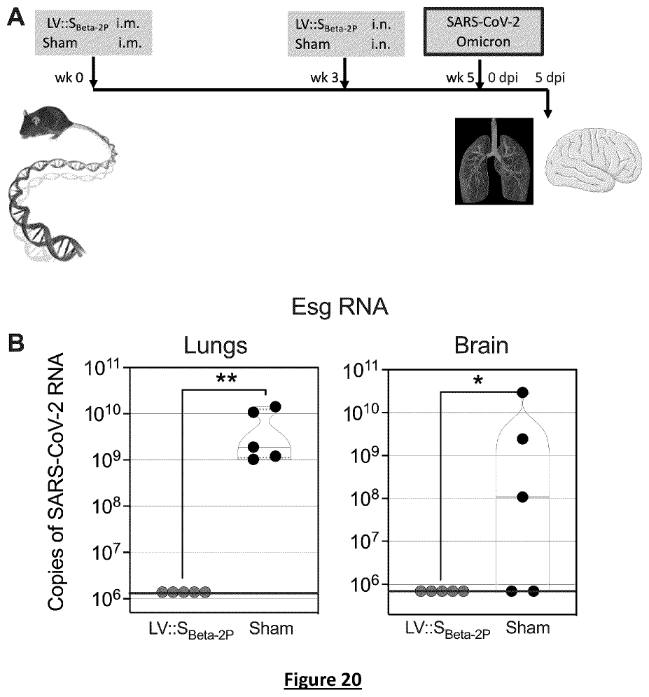

The inventors evaluated the protective efficacy of LV::S_{Beta-2P} in B6.K18-hACE2^{IP-THV} transgenic mice, which were prone to SARS-CoV-2 infection in the lung, and in addition displayed unprecedented brain permissiveness to SARS-CoV-2 replication (Ku M W, et al. EMBO Mol Med, e14459, 2021). B6.K18-hACE2^{IP-THV} mice (n=5/group) were primed intramuscularly (i.m.) with 1×10⁸ TU/mouse of LV::S_{Beta-2P} or an empty LV (sham) at wk 0 and then boosted intranasally (i.n.) at wk 3 with the same dose of the same vectors (FIG. 20A). Mice were then challenged (i.n.) with 0.3×10⁵ TCID₅₀ of SARS-CoV-2 Omicron variant at wk 5.

Lung and brain viral RNA contents were then determined at day 5 post infection by using a sub-genomic E_{CoV-2} RNA (Esg) qRT-PCR, which is an indicator of active viral replication (Chandrashekar et al., 2020, Science, 369, 812-817; Tostanoski et al., 2020, Nat. Med. 26, 1694-1700; Wolfel et al., 2020, Nature, 581, 465-469). LV::S_{Beta-2P} vaccination conferred sterilizing protection against SARS-CoV-2 Omicron in the lungs, i.e., undetectable Esg viral RNA in the vaccinated mice versus (5.83±6.22)×10⁹ copies of viral RNA/lungs in their sham-vaccinated counterparts (FIG. 20B, left). In parallel, Esg qRT-PCR quantification of viral RNA contents in brain detected no copies of viral RNA in the vaccinated mice versus (6.41±1.29)×10⁹ copies in the brain of the sham-vaccinated controls (FIG. 20B right). Of note, even if 2 mice out of 5 did not show cervical infection with Omicron, the three others showed very high amounts of viral replication in the brain.

Therefore the LV:S_{Beta-2P} displayed a full cross-protective capacity against the Omicron variant, which was fully comparable to its efficiency against the ancestral or the Delta variant that we previously demonstrated (Ku M W, et al. EMBO Mol Med, e14459, 2021; Ku M W, et al. Cell Host Microbe, 29(2), 236-249 e236, 2021; Vesin, B., et al. Mol Ther 30, 2984-2997, 2022).

1-18. (canceled)

19. A method for heterologous boosting in a vaccine regimen comprising administrating a pseudotyped lentiviral vector particle encoding a Spike (S) protein of a Severe Acute Respiratory Syndrome coronavirus 2 (SARS-CoV-2) to the upper respiratory tract of a human subject who has received a prime immunization with a SARS-CoV-2 vaccine composition selected from the group consisting of a protein, an mRNA, an adenovirus, an inactivated virus and a protein subunit vaccine composition.

20. The method of claim 19, wherein the administration is performed as intranasal mucosal boost immunization.

21. The method of claim 19, wherein the S protein of SARS-CoV-2 is (i) an S protein from a SARS-CoV-2 virus selected from the group consisting of SARS-CoV-2 Ancestral strain, D614G strain, Alpha strain, Beta strain, Gamma strain, Delta strain and Omicron strain or (ii) an S protein from a variant of said Ancestral, D614G, Alpha, Beta, Gamma, Delta or Omicron strains, wherein such variant encodes a Spike protein with an amino acid sequence at least 90% identity to SEQ ID NO: 1.

22. The method of claim 19, wherein S protein of SARS-CoV-2 is (a) a stabilized form of the S protein characterized by substitution of two consecutive amino acid residues for Proline residues in the S2 domain of the S protein at positions provided as residues 986 and 987 by reference to SEQ ID NO: 1 in the amino acid sequence of the Spike protein or (b) is a prefusion form of the S protein by deletion of the furin site located from residue 675 to residue 685 by reference to SEQ ID NO: 1 in the amino acid sequence of the S protein or (c) is a stabilized prefusion form of the S protein by deletion of the furin site and substitution of two consecutive amino acid residues for Proline residues in the S2 domain at positions 986 and 987 by reference to SEQ ID NO: 1.

23. The method of claim 19, wherein the amino acid sequence of the S protein of SARS-CoV-2 is SEQ ID NO: 1 or an amino acid sequence having at least 90% identity to SEQ ID NO: 1, and wherein the S protein of SARS-CoV-2 comprises at least five amino acid mutations including (i) a mutation of the lysine residue to the asparagine residue at position 417 of the amino acid sequence of SEQ ID NO: 1 (K417N), (ii) a mutation of the glutamic acid residue to the lysine residue at position 484 of the amino acid sequence of SEQ ID NO: 1 (E484K) or a mutation of the glutamic acid residue to the alanine residue at position 484 of the amino acid sequence of SEQ ID NO: 1 (E484A), (iii) a mutation of the asparagine residue to the tyrosine residue at position 501 of the amino acid sequence of SEQ ID NO: 1 (N501Y), (iv) a mutation of the lysine residue to the proline residue at position 986 of the amino acid sequence of SEQ ID NO: 1 (K986P) and (v) a mutation of the valine residue to the proline residue at position 987 of the amino acid sequence of SEQ ID NO: 1 (V987P).

24. The method of claim 19, wherein the subject receiving administration of the composition is a subject that has received a an intramuscular, intradermal or sub-cutaneous prime administration of a protein- or an mRNA-based vaccine composition against SARS-CoV-2 infection or disease.

25. The method of claim 23, wherein the S protein of SARS-CoV-2 further comprises amino acid mutations selected from the group consisting of (vi) a mutation of the glycine residue to the serine residue at position 446 of the amino acid sequence of SEQ ID NO: 1 (G446S), (vii) a mutation of the threonine residue to the lysine residue at position 478 of the amino acid sequence of SEQ ID NO: 1 (T478K), (viii) a mutation of the glutamine residue to the arginine residue at position 493 of the amino acid sequence of SEQ ID NO: 1 (Q493R) and (ix) a mutation of the glutamine residue to the arginine residue at position 498 of the amino acid sequence of SEQ ID NO: 1 (Q498R).

26. The method of claim 19, wherein the S protein of SARS-CoV-2 has the amino acid sequence of SEQ ID NO: 10 or SEQ ID NO: 18.

27. The method of claim 19, wherein the pseudotyped lentiviral vector particle is pseudotyped with a Vesicular Stomatitis Virus glycoprotein G (VSV-G) protein.

28. The method of claim 19, wherein the pseudotyped lentiviral vector particle elicits a CD8⁺ T-cell response against SARS-CoV-2.

29. The method of claim 28, wherein the pseudotyped lentiviral vector particle elicits lung-resident memory CD8⁺ T cells (Trm) and/or effector CD8⁺ T cells (Tc1) specific to Spike and produce Interferon-gamma (IFN-γ)/Tumor Necrosis Factor (TNF)/Interleukin-2 (IL-2).

30. The method of claim 19, wherein the pseudotyped lentiviral vector particle is non-integrative, non-cytopathic and non-replicative.

31. The method of claim 19, wherein the subject has a waning immunity from week 12 after the first injection of the initial vaccination with a vaccine composition against SARS-CoV-2 infection or disease.

32. The method of claim 19, wherein the pseudotyped lentiviral vector particle is formulated as a liquid composition or a dry powder for an administration as intranasal aerosols, intranasal drops or intranasal insufflations.

33. The method of claim 19, wherein the administration regimen comprises administration of one or more dosage form(s) of the pseudotyped lentiviral vector particle, wherein the dose of each dosage form is from 10⁷ to 10⁹ Transduction Unit (TU).

34. The method of claim 19, wherein the prime immunization is with protein SARS-CoV-2 vaccine composition.

35. The method of claim 19, wherein the prime immunization is with an mRNA SARS-CoV-2 vaccine composition.

36. An immunogenic composition comprising a pseudotyped lentiviral vector particle encoding an S protein of a SARS-CoV-2 and a pharmaceutically acceptable carrier, wherein the S protein of SARS-CoV-2 comprises at least nine amino acid mutations including (i) a mutation of the lysine residue to the asparagine residue at position 417 of the amino acid sequence of SEQ ID NO: 1 (K417N), (ii) a mutation of the glutamic acid residue to the alanine residue at position 484 of the amino acid sequence of SEQ ID NO: 1 (E484A), (iii) a mutation of the asparagine residue to the tyrosine residue at position 501 of the amino acid sequence of SEQ ID NO: 1 (N501Y), (iv) a mutation of the lysine residue to the proline residue at position 986 of the amino acid sequence of SEQ ID NO: 1 (K986P), (v) a mutation of the valine residue to the proline residue at position 987 of the amino acid sequence of SEQ ID NO: 1 (V987P), (vi) a mutation of the glycine residue to the serine residue at position 446 of the amino acid sequence of SEQ ID NO: 1 (G446S), (vii) a mutation of the threonine residue to the lysine residue at position 478 of the amino acid sequence of SEQ ID NO: 1 (T478K), (viii) a mutation of the glutamine residue to the arginine residue at position 493 of the amino acid sequence of SEQ ID NO: 1 (Q493R) and (ix) a mutation of the glutamine residue to the arginine residue at position 498 of the amino acid sequence of SEQ ID NO: 1 (Q498R).

37. The immunogenic composition according to claim 36, wherein the S protein of SARS-CoV-2 has the amino acid sequence of SEQ ID NO: 18.

38. The immunogenic composition according to claim 37, which is formulated for intranasal administration.


SEQUENCE LISTING (27 sequences)

SEQ ID NO: 1 - protein, 1273 aa, synthetic construct
  REGION 1..1273: note = S-Ancestral protein of SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - DNA, 3822 bp, synthetic construct
  misc_feature 1..3822: note = Nucleotide sequence of the polynucleotide encoding S-Ancestral protein of SARS-CoV-2
  source 1..3822: mol_type = other DNA
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 3 - DNA, 3825 bp, synthetic construct
  misc_feature 1..3825: note = Nucleotide sequence of the polynucleotide encoding S-D614G-2P protein of SARS-CoV-2
  source 1..3825: mol_type = other DNA
atgttcgtgtttctggtgctgctgccactggtgtccagtcagtgcgtgaacctgaccacacgaacacagctgccaccagcctacaccaatagcttcacccgcggagtgtactaccccgacaaggtgttccgcagcagcgtgctgcatagcacccaggatctgtttctgcccttcttcagcaacgtgacctggttccacgccatccacgtgtccggcaccaatggcaccaagcgcttcgataatcccgtgctgcccttcaacgatggcgtgtactttgccagcaccgagaagtccaatatcatccgcggctggatcttcggcaccacactggatagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgcgtgtacagcagcgccaacaactgcaccttcgagtacgtgtcccagccattcctgatggacctggaaggcaagcagggcaacttcaagaacctgcgcgagttcgtgttcaagaacatcgacggctacttcaaaatctacagcaagcacaccccaatcaacctcgtgcgcgatctgccacagggattcagtgctctggaacccctggtggatctgcccatcggcatcaacattacccgctttcagacactgctggccctgcaccgcagttacttgacaccaggcgatagcagcagtggatggacagctggtgccgccgcttactacgttggatatctgcagccacgcacctttctgctgaagtacaacgagaacggcaccatcaccgacgccgtggattgtgctctcgatcccctgagcgagacaaagtgcaccctgaagtccttcaccgtcgagaagggcatctaccagaccagcaatttccgcgtgcagcccaccgagagcatcgtgcgcttccccaatatcaccaatctgtgccccttcggcgaggtgttcaatgccacacgctttgcctccgtgtacgcctggaatcgcaagcgcattagcaactgcgtggccgactactccgtgctgtacaatagcgccagcttcagcaccttcaagtgctacggcgtgtcacccaccaagctgaacgacctgtgcttcaccaatgtgtacgccgacagcttcgtgatccgcggagatgaagtgcgacagattgccccaggccagaccggcaagatcgccgactacaattacaagctgcccgacgacttcaccggctgcgtgatcgcctggaacagcaacaacctggattccaaagtcggcggcaactacaactacctgtaccgcctgttccgcaagagcaatctgaagcccttcgagcgcgacatcagcaccgaaatctaccaggccggaagcaccccatgcaacggcgtggaaggcttcaactgctacttcccactgcagtcctacggatttcagcccacaaatggcgtgggctaccagccatatcgagtggtggtgctgagcttcgaactgctgcatgctccagctaccgtgtgcggccccaagaagagtaccaacctggtcaagaacaaatgcgtgaacttcaacttcaacggcctgaccggaaccggcgtgctgaccgagagtaacaagaagttcctgccattccagcagtttggccgcgacattgccgatacaaccgatgccgttcgcgatccccagaccttggagatcctggatattaccccatgctccttcggcggcgtgtccgtgattacaccaggcaccaataccagcaaccaggtggccgttctgtaccagggcgtgaattgcacagaggtgcccgtggccattcacgccgatcaattgacaccaacatggcgcgtgtactccaccggcagcaatgtgtttcaaacccgcgctggatgcctgattggagccgagcacgtgaacaatagctacgagtgcgatatccccatcggagccggaatctgcgcctcctatcagacccagaccaatagtccacgacgagcccgaagtgtggccagccagagcatcattgcctataccatgagcctgggcgccgagaatagcgtggcctactccaacaacagcattgctatccccaccaacttcaccatcagcgtgaccaccgagatcctgccagtgtccatgaccaagaccagcgtggactgcaccatgtacatctgcggagatagcaccgagtgcagcaacctgctgctgcagtacggaagtttctgcacccagctgaatcgcgccctgacaggcattgccgtggaacaggataagaacacccaagaggtgttcgcccaagtgaagcaaatctacaagaccccaccaatcaaggatttcggcggcttcaatttcagccagattctgcccgatccaagcaagcccagcaagcgcagcttcatcgaggacctgctgttcaacaaagtgacactggccgacgccggattcatcaagcagtatggcgattgcctgggcgatattgccgcacgcgatctgatttgcgcccagaagtttaacggactgaccgtcctgccaccactgctgacagatgagatgatcgcccagtacacaagtgccctgctggccggaaccattaccagcggatggacatttggagccggtgccgctctgcagattcccttcgctatgcagatggcctaccgcttcaatggcattggcgtgacccagaatgtgctgtacgagaaccagaagctgatcgccaaccagttcaacagcgccatcggcaagattcaggacagcctgagtagtaccgccagcgctctgggaaagctgcaggatgtggtcaaccagaacgctcaggccctgaacaccctggttaagcagctgagcagcaacttcggcgccatcagtagcgtgctgaacgatatcctgagccgcctggatccgccggaagccgaggtgcagatcgatcgcctgattaccggacgcctgcagtccctgcagacctatgtgacacagcagctgatccgagccgccgagattcgagctagtgctaatctggccgccaccaagatgagcgaatgtgtgctgggacagagcaagcgcgtggacttttgcggcaagggataccacctgatgagcttcccacagagtgctccacacggcgtggtgtttctgcatgtgacctacgtgcccgctcaagagaagaatttcaccaccgctccagccatctgccacgacggaaaggcccattttccacgcgagggcgtgttcgttagcaacggcactcattggttcgtcacccagcgcaacttctacgagccccagatcatcaccaccgacaacaccttcgtcagcggcaactgcgacgtcgtgatcggcattgtgaacaacaccgtgtacgatccactgcagcccgagctggacagcttcaaagaggaactggacaagtactttaagaaccacacaagccccgacgtggacctgggagacattagcggaatcaacgccagcgtggtcaacatccagaaagagattgaccgcctgaacgaggtggccaagaatctgaacgagagcctgatcgacctgcaagaactgggcaaatacgagcagtacattaagtggccctggtacatctggctgggcttcattgccggactgattgccatcgtgatggtcaccattatgctgtgctgcatgaccagttgctgcagctgcctgaagggatgctgcagttgcggaagctgctgcaagttcgacgaggatgatagcgagccagtgctgaagggcgtcaagctgcactacacctgataa

SEQ ID NO: 4 - protein, 1273 aa, synthetic construct
  REGION 1..1273: note = S-D614G-2P protein of SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 5 - DNA, 3813 bp, synthetic construct
  misc_feature 1..3813: note = Nucleotide sequence of the polynucleotide encoding S-Alpha-2P protein of SARS-CoV-2
  source 1..3813: mol_type = other DNA
atgttcgtgtttctggtgctgctgccactggtgtccagtcagtgcgtgaacctgaccacacgaacacagctgccaccagcctacaccaatagcttcacccgcggagtgtactaccccgacaaggtgttccgcagcagcgtgctgcatagcacccaggatctgtttctgcccttcttcagcaacgtgacctggttccacgccatctccggcaccaatggcaccaagcgcttcgataatcccgtgctgcccttcaacgatggcgtgtactttgccagcaccgagaagtccaatatcatccgcggctggatcttcggcaccacactggatagcaagacccagagcctgctgatcgtgaacaacgccaccaacgtggtcatcaaagtgtgcgagttccagttctgcaacgaccccttcctgggcgtctaccacaagaacaacaagagctggatggaaagcgagttccgcgtgtacagcagcgccaacaactgcaccttcgagtacgtgtcccagccattcctgatggacctggaaggcaagcagggcaacttcaagaacctgcgcgagttcgtgttcaagaacatcgacggctacttcaaaatctacagcaagcacaccccaatcaacctcgtgcgcgatctgccacagggattcagtgctctggaacccctggtggatctgcccatcggcatcaacattacccgctttcagacactgctggccctgcaccgcagttacttgacaccaggcgatagcagcagtggatggacagctggtgccgccgcttactacgttggatatctgcagccacgcacctttctgctgaagtacaacgagaacggcaccatcaccgacgccgtggattgtgctctcgatcccctgagcgagacaaagtgcaccctgaagtccttcaccgtcgagaagggcatctaccagaccagcaatttccgcgtgcagcccaccgagagcatcgtgcgcttccccaatatcaccaatctgtgccccttcggcgaggtgttcaatgccacacgctttgcctccgtgtacgcctggaatcgcaagcgcattagcaactgcgtggccgactactccgtgctgtacaatagcgccagcttcagcaccttcaagtgctacggcgtgtcacccaccaagctgaacgacctgtgcttcaccaatgtgtacgccgacagcttcgtgatccgcggagatgaagtgcgacagattgccccaggccagaccggcaagatcgccgactacaattacaagctgcccgacgacttcaccggctgcgtgatcgcctggaacagcaacaacctggattccaaagtcggcggcaactacaactacctgtaccgcctgttccgcaagagcaatctgaagcccttcgagcgcgacatcagcaccgaaatctaccaggccggaagcaccccatgcaacggcgtggaaggcttcaactgctacttcccactgcagtcctacggatttcagcccacatatggcgtgggctaccagccatatcgagtggtggtgctgagcttcgaactgctgcatgctccagctaccgtgtgcggccccaagaagagtaccaacctggtcaagaacaaatgcgtgaacttcaacttcaacggcctgaccggaaccggcgtgctgaccgagagtaacaagaagttcctgccattccagcagtttggccgcgacattgacgatacaaccgatgccgttcgcgatccccagaccttggagatcctggatattaccccatgctccttcggcggcgtgtccgtgattacaccaggcaccaataccagcaaccaggtggccgttctgtaccagggcgtgaattgcacagaggtgcccgtggccattcacgccgatcaattgacaccaacatggcgcgtgtactccaccggcagcaatgtgtttcaaacccgcgctggatgcctgattggagccgagcacgtgaacaatagctacgagtgcgatatccccatcggagccggaatctgcgcctcctatcagacccagaccaatagtcaccgacgagcccgaagtgtggccagccagagcatcattgcctataccatgagcctgggcgccgagaatagcgtggcctactccaacaacagcattgctatccccatcaacttcaccatcagcgtgaccaccgagatcctgccagtgtccatgaccaagaccagcgtggactgcaccatgtacatctgcggagatagcaccgagtgcagcaacctgctgctgcagtacggaagtttctgcacccagctgaatcgcgccctgacaggcattgccgtggaacaggataagaacacccaagaggtgttcgcccaagtgaagcaaatctacaagaccccaccaatcaaggatttcggcggcttcaatttcagccagattctgcccgatccaagcaagcccagcaagcgcagcttcatcgaggacctgctgttcaacaaagtgacactggccgacgccggattcatcaagcagtatggcgattgcctgggcgatattgccgcacgcgatctgatttgcgcccagaagtttaacggactgaccgtcctgccaccactgctgacagatgagatgatcgcccagtacacaagtgccctgctggccggaaccattaccagcggatggacatttggagccggtgccgctctgcagattcccttcgctatgcagatggcctaccgcttcaatggcattggcgtgacccagaatgtgctgtacgagaaccagaagctgatcgccaaccagttcaacagcgccatcggcaagattcaggacagcctgagtagtaccgccagcgctctgggaaagctgcaggatgtggtcaaccagaacgctcaggccctgaacaccctggttaagcagctgagcagcaacttcggcgccatcagtagcgtgctgaacgatatcctggcccgcctggatccgccggaagccgaggtgcagatcgatcgcctgattaccggacgcctgcagtccctgcagacctatgtgacacagcagctgatccgagccgccgagattcgagctagtgctaatctggccgccaccaagatgagcgaatgtgtgctgggacagagcaagcgcgtggacttttgcggcaagggataccacctgatgagcttcccacagagtgctccacacggcgtggtgtttctgcatgtgacctacgtgcccgctcaagagaagaatttcaccaccgctccagccatctgccacgacggaaaggcccattttccacgcgagggcgtgttcgttagcaacggcactcattggttcgtcacccagcgcaacttctacgagccccagatcatcaccacccacaacaccttcgtcagcggcaactgcgacgtcgtgatcggcattgtgaacaacaccgtgtacgatccactgcagcccgagctggacagcttcaaagaggaactggacaagtactttaagaaccacacaagccccgacgtggacctgggagacattagcggaatcaacgccagcgtggtcaacatccagaaagagattgaccgcctgaacgaggtggccaagaatctgaacgagagcctgatcgacctgcaagaactgggcaaatacgagcagtacattaagtggccctggtacatctggctgggcttcattgccggactgattgccatcgtgatggtcaccattatgctgtgctgcatgaccagttgctgcagctgcctgaagggatgctgcagttgcggaagctgctgcaagttcgacgaggatgatagcgagccagtgctgaagggcgtcaagctgcactacacctga

SEQ ID NO: 6 - protein, 1270 aa, synthetic construct
  REGION 1..1270: note = S-Alpha-2P protein of SARS-CoV-2
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILARLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 7 - DNA, 3813 bp, synthetic construct
  misc_feature 1..3813: note = Nucleotide sequence of the polynucleotide encoding S-Beta protein of SARS-CoV-2
  source 1..3813: mol_type = other DNA
atgttcgtgtttctggtgctgctgccactggtgtccagtcagtgcgtgaacttcaccacacgaacacagctgccaccagcctacaccaatagcttcacccgcggagtgtactaccccgacaaggtgttccgcagcagcgtgctgcatagcacccaggatctgtttctgcccttcttcagcaacgtgacctggttccacgccatccacgtgtccggcaccaatggcaccaagcgcttcgctaatcccgtgctgcccttcaacgatggcgtgtactttgccagcaccgagaagtccaatatcatccgcggctggatcttcggcaccacactggatagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgcgtgtacagcagcgccaacaactgcaccttcgagtacgtgtcccagccattcctgatggacctggaaggcaagcagggcaacttcaagaacctgcgcgagttcgtgttcaagaacatcgacggctacttcaaaatctacagcaagcacaccccaatcaacctcgtgcgcggcctgccacagggattcagtgctctggaacccctggtggatctgcccatcggcatcaacattacccgctttcagacactgcaccgcagttacttgacaccaggcgatagcagcagtggatggacagctggtgccgccgcttactacgttggatatctgcagccacgcacctttctgctgaagtacaacgagaacggcaccatcaccgacgccgtggattgtgctctcgatcccctgagcgagacaaagtgcaccctgaagtccttcaccgtcgagaagggcatctaccagaccagcaatttccgcgtgcagcccaccgagagcatcgtgcgcttccccaatatcaccaatctgtgccccttcggcgaggtgttcaatgccacacgctttgcctccgtgtacgcctggaatcgcaagcgcattagcaactgcgtggccgactactccgtgctgtacaatagcgccagcttcagcaccttcaagtgctacggcgtgtcacccaccaagctgaacgacctgtgcttcaccaatgtgtacgccgacagcttcgtgatccgcggagatgaagtgcgacagattgccccaggccagaccggcaacatcgccgactacaattacaagctgcccgacgacttcaccggctgcgtgatcgcctggaacagcaacaacctggattccaaagtcggcggcaactacaactacctgtaccgcctgttccgcaagagcaatctgaagcccttcgagcgcgacatcagcaccgaaatctaccaggccggaagcaccccatgcaacggcgtgaaaggcttcaactgctacttcccactgcagtcctacggatttcagcccacatatggcgtgggctaccagccatatcgagtggtggtgctgagcttcgaactgctgcatgctccagctaccgtgtgcggccccaagaagagtaccaacctggtcaagaacaaatgcgtgaacttcaacttcaacggcctgaccggaaccggcgtgctgaccgagagtaacaagaagttcctgccattccagcagtttggccgcgacattgccgatacaaccgatgccgttcgcgatccccagaccttggagatcctggatattaccccatgctccttcggcggcgtgtccgtgattacaccaggcaccaataccagcaaccaggtggccgttctgtaccagggcgtgaattgcacagaggtgcccgtggccattcacgccgatcaattgacaccaacatggcgcgtgtactccaccggcagcaatgtgtttcaaacccgcgctggatgcctgattggagccgagcacgtgaacaatagctacgagtgcgatatccccatcggagccggaatctgcgcctcctatcagacccagaccaatagtccacgacgagcccgaagtgtggccagccagagcatcattgcctataccatgagcctgggcgtggagaatagcgtggcctactccaacaacagcattgctatccccaccaacttcaccatcagcgtgaccaccgagatcctgccagtgtccatgaccaagaccagcgtggactgcaccatgtacatctgcggagatagcaccgagtgcagcaacctgctgctgcagtacggaagtttctgcacccagctgaatcgcgccctgacaggcattgccgtggaacaggataagaacacccaagaggtgttcgcccaagtgaagcaaatctacaagaccccaccaatcaaggatttcggcggcttcaatttcagccagattctgcccgatccaagcaagcccagcaagcgcagcttcatcgaggacctgctgttcaacaaagtgacactggccgacgccggattcatcaagcagtatggcgattgcctgggcgatattgccgcacgcgatctgatttgcgcccagaagtttaacggactgaccgtcctgccaccactgctgacagatgagatgatcgcccagtacacaagtgccctgctggccggaaccattaccagcggatggacatttggagccggtgccgctctgcagattcccttcgctatgcagatggcctaccgcttcaatggcattggcgtgacccagaatgtgctgtacgagaaccagaagctgatcgccaaccagttcaacagcgccatcggcaagattcaggacagcctgagtagtaccgccagcgctctgggaaagctgcaggatgtggtcaaccagaacgctcaggccctgaacaccctggttaagcagctgagcagcaacttcggcgccatcagtagcgtgctgaacgatatcctgagccgcctggataaggtggaagccgaggtgcagatcgatcgcctgattaccggacgcctgcagtccctgcagacctatgtgacacagcagctgatccgagccgccgagattcgagctagtgctaatctggccgccaccaagatgagcgaatgtgtgctgggacagagcaagcgcgtggacttttgcggcaagggataccacctgatgagcttcccacagagtgctccacacggcgtggtgtttctgcatgtgacctacgtgcccgctcaagagaagaatttcaccaccgctccagccatctgccacgacggaaaggcccattttccacgcgagggcgtgttcgttagcaacggcactcattggttcgtcacccagcgcaacttctacgagccccagatcatcaccaccgacaacaccttcgtcagcggcaactgcgacgtcgtgatcggcattgtgaacaacaccgtgtacgatccactgcagcccgagctggacagcttcaaagaggaactggacaagtactttaagaaccacacaagccccgacgtggacctgggagacattagcggaatcaacgccagcgtggtcaacatccagaaagagattgaccgcctgaacgaggtggccaagaatctgaacgagagcctgatcgacctgcaagaactgggcaaatacgagcagtacattaagtggccctggtacatctggctgggcttcattgccggactgattgccatcgtgatggtcaccattatgctgtgctgcatgaccagttgctgcagctgcctgaagggatgctgcagttgcggaagctgctgcaagttcgacgaggatgatagcgagccagtgctgaagggcgtcaagctgcactacacctga

SEQ ID NO: 8 - protein, 1270 aa, synthetic construct
  REGION 1..1270: note = S-Beta protein of SARS-CoV-2
MFVFLVLLPLVSSQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 9 - DNA, 3813 bp, synthetic construct
  misc_feature 1..3813: note = Nucleotide sequence of the polynucleotide encoding S-Beta-2P protein of SARS-CoV-2
  source 1..3813: mol_type = other DNA
atgttcgtgtttctggtgctgctgccactggtgtccagtcagtgcgtgaacttcaccacacgaacacagctgccaccagcctacaccaatagcttcacccgcggagtgtactaccccgacaaggtgttccgcagcagcgtgctgcatagcacccaggatctgtttctgcccttcttcagcaacgtgacctggttccacgccatccacgtgtccggcaccaatggcaccaagcgcttcgctaatcccgtgctgcccttcaacgatggcgtgtactttgccagcaccgagaagtccaatatcatccgcggctggatcttcggcaccacactggatagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgcgtgtacagcagcgccaacaactgcaccttcgagtacgtgtcccagccattcctgatggacctggaaggcaagcagggcaacttcaagaacctgcgcgagttcgtgttcaagaacatcgacggctacttcaaaatctacagcaagcacaccccaatcaacctcgtgcgcggcctgccacagggattcagtgctctggaacccctggtggatctgcccatcggcatcaacattacccgctttcagacactgcaccgcagttacttgacaccaggcgatagcagcagtggatggacagctggtgccgccgcttactacgttggatatctgcagccacgcacctttctgctgaagtacaacgagaacggcaccatcaccgacgccgtggattgtgctctcgatcccctgagcgagacaaagtgcaccctgaagtccttcaccgtcgagaagggcatctaccagaccagcaatttccgcgtgcagcccaccgagagcatcgtgcgcttccccaatatcaccaatctgtgccccttcggcgaggtgttcaatgccacacgctttgcctccgtgtacgcctggaatcgcaagcgcattagcaactgcgtggccgactactccgtgctgtacaatagcgccagcttcagcaccttcaagtgctacggcgtgtcacccaccaagctgaacgacctgtgcttcaccaatgtgtacgccgacagcttcgtgatccgcggagatgaagtgcgacagattgccccaggccagaccggcaacatcgccgactacaattacaagctgcccgacgacttcaccggctgcgtgatcgcctggaacagcaacaacctggattccaaagtcggcggcaactacaactacctgtaccgcctgttccgcaagagcaatctgaagcccttcgagcgcgacatcagcaccgaaatctaccaggccggaagcaccccatgcaacggcgtgaaaggcttcaactgctacttcccactgcagtcctacggatttcagcccacatatggcgtgggctaccagccatatcgagtggtggtgctgagcttcgaactgctgcatgctccagctaccgtgtgcggccccaagaagagtaccaacctggtcaagaacaaatgcgtgaacttcaacttcaacggcctgaccggaaccggcgtgctgaccgagagtaacaagaagttcctgccattccagcagtttggccgcgacattgccgatacaaccgatgccgttcgcgatccccagaccttggagatcctggatattaccccatgctccttcggcggcgtgtccgtgattacaccaggcaccaataccagcaaccaggtggccgttctgtaccagggcgtgaattgcacagaggtgcccgtggccattcacgccgatcaattgacaccaacatggcgcgtgtactccaccggcagcaatgtgtttcaaacccgcgctggatgcctgattggagccgagcacgtgaacaatagctacgagtgcgatatccccatcggagccggaatctgcgcctcctatcagacccagaccaatagtccacgacgagcccgaagtgtggccagccagagcatcattgcctataccatgagcctgggcgtggagaatagcgtggcctactccaacaacagcattgctatccccaccaacttcaccatcagcgtgaccaccgagatcctgccagtgtccatgaccaagaccagcgtggactgcaccatgtacatctgcggagatagcaccgagtgcagcaacctgctgctgcagtacggaagtttctgcacccagctgaatcgcgccctgacaggcattgccgtggaacaggataagaacacccaagaggtgttcgcccaagtgaagcaaatctacaagaccccaccaatcaaggatttcggcggcttcaatttcagccagattctgcccgatccaagcaagcccagcaagcgcagcttcatcgaggacctgctgttcaacaaagtgacactggccgacgccggattcatcaagcagtatggcgattgcctgggcgatattgccgcacgcgatctgatttgcgcccagaagtttaacggactgaccgtcctgccaccactgctgacagatgagatgatcgcccagtacacaagtgccctgctggccggaaccattaccagcggatggacatttggagccggtgccgctctgcagattcccttcgctatgcagatggcctaccgcttcaatggcattggcgtgacccagaatgtgctgtacgagaaccagaagctgatcgccaaccagttcaacagcgccatcggcaagattcaggacagcctgagtagtaccgccagcgctctgggaaagctgcaggatgtggtcaaccagaacgctcaggccctgaacaccctggttaagcagctgagcagcaacttcggcgccatcagtagcgtgctgaacgatatcctgagccgcctggatccgccggaagccgaggtgcagatcgatcgcctgattaccggacgcctgcagtccctgcagacctatgtgacacagcagctgatccgagccgccgagattcgagctagtgctaatctggccgccaccaagatgagcgaatgtgtgctgggacagagcaagcgcgtggacttttgcggcaagggataccacctgatgagcttcccacagagtgctccacacggcgtggtgtttctgcatgtgacctacgtgcccgctcaagagaagaatttcaccaccgctccagccatctgccacgacggaaaggcccattttccacgcgagggcgtgttcgttagcaacggcactcattggttcgtcacccagcgcaacttctacgagccccagatcatcaccaccgacaacaccttcgtcagcggcaactgcgacgtcgtgatcggcattgtgaacaacaccgtgtacgatccactgcagcccgagctggacagcttcaaagaggaactggacaagtactttaagaaccacacaagccccgacgtggacctgggagacattagcggaatcaacgccagcgtggtcaacatccagaaagagattgaccgcctgaacgaggtggccaagaatctgaacgagagcctgatcgacctgcaagaactgggcaaatacgagcagtacattaagtggccctggtacatctggctgggcttcattgccggactgattgccatcgtgatggtcaccattatgctgtgctgcatgaccagttgctgcagctgcctgaagggatgctgcagttgcggaagctgctgcaagttcgacgaggatgatagcgagccagtgctgaagggcgtcaagctgcactacacctga

SEQ ID NO: 10 - protein, 1270 aa, synthetic construct
  REGION 1..1270: note = S-Beta-2P protein of SARS-CoV-2
MFVFLVLLPLVSSQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 11 - DNA, 3822 bp, synthetic construct
  misc_feature 1..3822: note = Nucleotide sequence of the polynucleotide encoding S-Gamma-2P protein of SARS-CoV-2
  source 1..3822: mol_type = other DNA
atgttcgtgtttctggtgctgctgccactggtgtccagtcagtgcgtgaacttcaccaaccgaacacagctgccatccgcctacaccaatagcttcacccgcggagtgtactaccccgacaaggtgttccgcagcagcgtgctgcatagcacccaggatctgtttctgcccttcttcagcaacgtgacctggttccacgccatccacgtgtccggcaccaatggcaccaagcgcttcgataatcccgtgctgcccttcaacgatggcgtgtactttgccagcaccgagaagtccaatatcatccgcggctggatcttcggcaccacactggatagcaagacccagagcctgctgatcgtgaacaacgccaccaacgtggtcatcaaagtgtgcgagttccagttctgcaactaccccttcctgggcgtctactaccacaagaacaacaagagctggatggaaagcgagttccgcgtgtacagcagcgccaacaactgcaccttcgagtacgtgtcccagccattcctgatggacctggaaggcaagcagggcaacttcaagaacctgtccgagttcgtgttcaagaacatcgacggctacttcaaaatctacagcaagcacaccccaatcaacctcgtgcgcgatctgccacagggattcagtgctctggaacccctggtggatctgcccatcggcatcaacattacccgctttcagacactgctggccctgcaccgcagttacttgacaccaggcgatagcagcagtggatggacagctggtgccgccgcttactacgttggatatctgcagccacgcacctttctgctgaagtacaacgagaacggcaccatcaccgacgccgtggattgtgctctcgatcccctgagcgagacaaagtgcaccctgaagtccttcaccgtcgagaagggcatctaccagaccagcaatttccgcgtgcagcccaccgagagcatcgtgcgcttccccaatatcaccaatctgtgccccttcggcgaggtgttcaatgccacacgctttgcctccgtgtacgcctggaatcgcaagcgcattagcaactgcgtggccgactactccgtgctgtacaatagcgccagcttcagcaccttcaagtgctacggcgtgtcacccaccaagctgaacgacctgtgcttcaccaatgtgtacgccgacagcttcgtgatccgcggagatgaagtgcgacagattgccccaggccagaccggcaccatcgccgactacaattacaagctgcccgacgacttcaccggctgcgtgatcgcctggaacagcaacaacctggattccaaagtcggcggcaactacaactacctgtaccgcctgttccgcaagagcaatctgaagcccttcgagcgcgacatcagcaccgaaatctaccaggccggaagcaccccatgcaacggcgtgaaaggcttcaactgctacttcccactgcagtcctacggatttcagcccacatatggcgtgggctaccagccatatcgagtggtggtgctgagcttcgaactgctgcatgctccagctaccgtgtgcggccccaagaagagtaccaacctggtcaagaacaaatgcgtgaacttcaacttcaacggcctgaccggaaccggcgtgctgaccgagagtaacaagaagttcctgccattccagcagtttggccgcgacattgccgatacaaccgatgccgttcgcgatccccagaccttggagatcctggatattaccccatgctccttcggcggcgtgtccgtgattacaccaggcaccaataccagcaaccaggtggccgttctgtaccagggcgtgaattgcacagaggtgcccgtggccattcacgccgatcaattgacaccaacatggcgcgtgtactccaccggcagcaatgtgtttcaaacccgcgctggatgcctgattggagccgagtacgtgaacaatagctacgagtgcgatatccccatcggagccggaatctgcgcctcctatcagacccagaccaatagtccacgacgagcccgaagtgtggccagccagagcatcattgcctataccatgagcctgggcgccgagaatagcgtggcctactccaacaacagcattgctatccccaccaacttcaccatcagcgtgaccaccgagatcctgccagtgtccatgaccaagaccagcgtggactgcaccatgtacatctgcggagatagcaccgagtgcagcaacctgctgctgcagtacggaagtttctgcacccagctgaatcgcgccctgacaggcattgccgtggaacaggataagaacacccaagaggtgttcgcccaagtgaagcaaatctacaagaccccaccaatcaaggatttcggcggcttcaatttcagccagattctgcccgatccaagcaagcccagcaagcgcagcttcatcgaggacctgctgttcaacaaagtgacactggccgacgccggattcatcaagcagtatggcgattgcctgggcgatattgccgcacgcgatctgatttgcgcccagaagtttaacggactgaccgtcctgccaccactgctgacagatgagatgatcgcccagtacacaagtgccctgctggccggaaccattaccagcggatggacatttggagccggtgccgctctgcagattcccttcgctatgcagatggcctaccgcttcaatggcattggcgtgacccagaatgtgctgtacgagaaccagaagctgatcgccaaccagttcaacagcgccatcggcaagattcaggacagcctgagtagtaccgccagcgctctgggaaagctgcaggatgtggtcaaccagaacgctcaggccctgaacaccctggttaagcagctgagcagcaacttcggcgccatcagtagcgtgctgaacgatatcctgagccgcctggatccgccggaagccgaggtgcagatcgatcgcctgattaccggacgcctgcagtccctgcagacctatgtgacacagcagctgatccgagccgccgagattcgagctagtgctaatctggccgccatcaagatgagcgaatgtgtgctgggacagagcaagcgcgtggacttttgcggcaagggataccacctgatgagcttcccacagagtgctccacacggcgtggtgtttctgcatgtgacctacgtgcccgctcaagagaagaatttcaccaccgctccagccatctgccacgacggaaaggcccattttccacgcgagggcgtgttcgttagcaacggcactcattggttcgtcacccagcgcaacttctacgagccccagatcatcaccaccgacaacaccttcgtcagcggcaactgcgacgtcgtgatcggcattgtgaacaacaccgtgtacgatccactgcagcccgagctggacagcttcaaagaggaactggacaagtactttaagaaccacacaagccccgacgtggacctgggagacattagcggaatcaacgccagcgtggtcaacatccagaaagagattgaccgcctgaacgaggtggccaagaatctgaacgagagcctgatcgacctgcaagaactgggcaaatacgagcagtacattaagtggccctggtacatctggctgggcttcattgccggactgattgccatcgtgatggtcaccattatgctgtgctgcatgaccagttgctgcagctgcctgaagggatgctgcagttgcggaagctgctgcaagttcgacgaggatgatagcgagccagtgctgaagggcgtcaagctgcactacacctga

SEQ ID NO: 12 - protein, 1273 aa, synthetic construct
  REGION 1..1273: note = S-Gamma-2P protein of SARS-CoV-2
MFVFLVLLPLVSSQCVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAAIKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 13 - DNA, 3816 bp, synthetic construct
  misc_feature 1..3816: note = Nucleotide sequence of the polynucleotide encoding S-Delta-2P protein of SARS-CoV-2
  source 1..3816: mol_type = other DNA
atgttcgtgtttctggtgctgctgccactggtgtccagtcagtgcgtgaacctgaggacacgaacacagctgccaccagcctacaccaatagcttcacccgcggagtgtactaccccgacaaggtgttccgcagcagcgtgctgcatagcacccaggatctgtttctgcccttcttcagcaacgtgacctggttccacgccatccacgtgtccggcaccaatggcaccaagcgcttcgataatcccgtgctgcccttcaacgatggcgtgtactttgccagcaccgagaagtccaatatcatccgcggctggatcttcggcaccacactggatagcaagacccagagcctgctgatcgtgaacaacgccaccaacgtggtcatcaaagtgtgcgagttccagttctgcaacgaccccttcctggacgtctactaccacaagaacaacaagagctggatggaaagcggcgtgtacagcagcgccaacaactgcaccttcgagtacgtgtcccagccattcctgatggacctggaaggcaagcagggcaacttcaagaacctgcgcgagttcgtgttcaagaacatcgacggctacttcaaaatctacagcaagcacaccccaatcaacctcgtgcgcgatctgccacagggattcagtgctctggaacccctggtggatctgcccatcggcatcaacattacccgctttcagacactgctggccctgcaccgcagttacttgacaccaggcgatagcagcagtggatggacagctggtgccgccgcttactacgttggatatctgcagccacgcacctttctgctgaagtacaacgagaacggcaccatcaccgacgccgtggattgtgctctcgatcccctgagcgagacaaagtgcaccctgaagtccttcaccgtcgagaagggcatctaccagaccagcaatttccgcgtgcagcccaccgagagcatcgtgcgcttccccaatatcaccaatctgtgccccttcggcgaggtgttcaatgccacacgctttgcctccgtgtacgcctggaatcgcaagcgcattagcaactgcgtggccgactactccgtgctgtacaatagcgccagcttcagcaccttcaagtgctacggcgtgtcacccaccaagctgaacgacctgtgcttcaccaatgtgtacgccgacagcttcgtgatccgcggagatgaagtgcgacagattgccccaggccagaccggcaagatcgccgactacaattacaagctgcccgacgacttcaccggctgcgtgatcgcctggaacagcaacaacctggattccaaagtcggcggcaactacaactacagataccgcctgttccgcaagagcaatctgaagcccttcgagcgcgacatcagcaccgaaatctaccaggccggaagcaagccatgcaacggcgtggaaggcttcaactgctacttcccactgcagtcctacggatttcagcccacaaatggcgtgggctaccagccatatcgagtggtggtgctgagcttcgaactgctgcatgctccagctaccgtgtgcggccccaagaagagtaccaacctggtcaagaacaaatgcgtgaacttcaacttcaacggcctgaccggaaccggcgtgctgaccgagagtaacaagaagttcctgccattccagcagtttggccgcgacattgccgatacaaccgatgccgttcgcgatccccagaccttggagatcctggatattaccccatgctccttcggcggcgtgtccgtgattacaccaggcaccaataccagcaaccaggtggccgttctgtaccagggcgtgaattgcacagaggtgcccgtggccattcacgccgatcaattgacaccaacatggcgcgtgtactccaccggcagcaatgtgtttcaaacccgcgctggatgcctgattggagccgagcacgtgaacaatagctacgagtgcgatatccccatcggagccggaatctgcgcctcctatcagacccagaccaatagtagacgacgagcccgaagtgtggccagccagagcatcattgcctataccatgagcctgggcgccgagaatagcgtggcctactccaacaacagcattgctatccccaccaacttcaccatcagcgtgaccaccgagatcctgccagtgtccatgaccaagaccagcgtggactgcaccatgtacatctgcggagatagcaccgagtgcagcaacctgctgctgcagtacggaagtttctgcacccagctgaatcgcgccctgacaggcattgccgtggaacaggataagaacacccaagaggtgttcgcccaagtgaagcaaatctacaagaccccaccaatcaaggatttcggcggcttcaatttcagccagattctgcccgatccaagcaagcccagcaagcgcagcttcatcgaggacctgctgttcaacaaagtgacactggccgacgccggattcatcaagcagtatggcgattgcctgggcgatattgccgcacgcgatctgatttgcgcccagaagtttaacggactgaccgtcctgccaccactgctgacagatgagatgatcgcccagtacacaagtgccctgctggccggaaccattaccagcggatggacatttggagccggtgccgctctgcagattcccttcgctatgcagatggcctaccgcttcaatggcattggcgtgacccagaatgtgctgtacgagaaccagaagctgatcgccaaccagttcaacagcgccatcggcaagattcaggacagcctgagtagtaccgccagcgctctgggaaagctgcagaacgtggtcaaccagaacgctcaggccctgaacaccctggttaagcagctgagcagcaacttcggcgccatcagtagcgtgctgaacgatatcctgagccgcctggatccgccggaagccgaggtgcagatcgatcgcctgattaccggacgcctgcagtccctgcagacctatgtgacacagcagctgatccgagccgccgagattcgagctagtgctaatctggccgccaccaagatgagcgaatgtgtgctgggacagagcaagcgcgtggacttttgcggcaagggataccacctgatgagcttcccacagagtgctccacacggcgtggtgtttctgcatgtgacctacgtgcccgctcaagagaagaatttcaccaccgctccagccatctgccacgacggaaaggcccattttccacgcgagggcgtgttcgttagcaacggcactcattggttcgtcacccagcgcaacttctacgagccccagatcatcaccaccgacaacaccttcgtcagcggcaactgcgacgtcgtgatcggcattgtgaacaacaccgtgtacgatccactgcagcccgagctggacagcttcaaagaggaactggacaagtactttaagaaccacacaagccccgacgtggacctgggagacattagcggaatcaacgccagcgtggtcaacatccagaaagagattgaccgcctgaacgaggtggccaagaatctgaacgagagcctgatcgacctgcaagaactgggcaaatacgagcagtacattaagtggccctggtacatctggctgggcttcattgccggactgattgccatcgtgatggtcaccattatgctgtgctgcatgaccagttgctgcagctgcctgaagggatgctgcagttgcggaagctgctgcaagttcgacgaggatgatagcgagccagtgctgaagggcgtcaagctgcactacacctga

SEQ ID NO: 14 - protein, 1271 aa, synthetic construct
  REGION 1..1271: note = S-Delta-2P protein of SARS-CoV-2
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 15 - DNA, 3813 bp, synthetic construct
  misc_feature 1..3813: note = Nucleotide sequence of the polynucleotide encoding S-Omicron protein of SARS-CoV-2
  source 1..3813: mol_type = other DNA
atgttcgtgtttctggtgctgctgcctctggtgtccagccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgtgatcagcggcaccaatggcaccaagagattcgacaaccccgtgctgcccttcaacgacggggtgtactttgccagcatcgagaagtccaacatcatccgcggctggatcttcggcaccacactggatagcaagacccagagcctgctgatcgtgaacaacgccaccaacgtggtcatcaaagtgtgcgagttccagttctgcaacgacccattcctggaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcatcgtgcgcgagcctgaggatctgcctcagggcttttctgccctggaacctctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctcttgatcccctgagcgagacaaagtgcaccctgaagtccttcaccgtggaaaagggcatctaccagaccagcaacttccgggtgcagcccaccgaatccatcgtgcggttccccaatatcaccaatctgtgccccttcgatgaggtgttcaatgccaccagattcgccagcgtgtacgcctggaaccggaagagaatcagcaactgcgtggccgactactccgtgctgtacaatctggccccattcttcaccttcaagtgctacggcgtgtcccctaccaagctgaacgacctgtgcttcaccaatgtgtacgccgacagcttcgtgatccggggagatgaagtgcggcagattgcccctggacagaccggcaatatcgccgactacaactacaagctgcccgacgacttcaccggctgtgtgatcgcctggaatagcaacaagctggacagcaaggtgtccggcaactacaattacctgtaccggctgttccggaagtccaatctgaagcccttcgagcgggacatcagcaccgagatctatcaggccggcaacaagccctgtaatggcgtggccggcttcaactgctacttcccactgcggagctacagcttcagacccacatacggcgttggccaccagccttacagagtggtggtgctgtccttcgagctgctgcatgctcctgccacagtgtgcggccctaagaaaagcaccaacctcgtgaagaacaaatgcgtgaacttcaacttcaacggcctgaaaggcaccggcgtgctgaccgagagcaacaagaagttcctgccattccagcagttcggccgggacattgccgataccacagatgctgtcagagatccccagacactggaaatcctggacatcaccccttgcagcttcggcggagtgtctgtgatcacccctggcaccaacaccagcaatcaggtggcagtgctgtaccagggcgtgaactgtacagaggtgccagtggccattcacgccgatcagctgacccctacttggcgggtgtactccacaggcagcaatgtgttccagaccagagccggctgtctgattggcgccgagtatgtgaacaacagctacgagtgcgacatccccatcggagccggcatctgtgccagctaccagacacagaccaagagccacagacgggctagaagcgtggccagccagagcatcattgcctacacaatgtctctgggcgccgagaacagcgtggcctacagcaacaactctatcgctatccccaccaacttcaccatcagcgtgaccaccgagattctgcccgtgtccatgaccaagaccagcgtggactgcaccatgtacatctgcggcgattccaccgagtgctccaacctgctgctgcagtacggcagcttctgcacccagctgaagagagccctgacagggattgccgtggaacaggacaagaacacccaagaggtgttcgcccaagtgaagcagatctacaagacccctcctatcaagtacttcggcgggttcaacttctcccagatcctgccagatcctagcaagcccagcaagcggagcttcatcgaggacctgctgttcaacaaagtgacactggccgacgccggctttatcaagcagtatggcgattgcctgggcgacattgcagccagggatctgatttgcgcccagaagttcaagggcctgacagtgctgcctcctctgctgacagatgagatgatcgcccagtacacaagcgccctgctggccggcacaatcacctctggatggacatttggagccggcgctgccctgcagatcccatttgctatgcagatggcctaccggttcaacggcatcggagtgacccagaatgtgctgtacgagaaccagaagctgatcgccaaccagttcaacagcgccatcggcaagatccaggacagcctgagcagcacagcctctgctctgggcaagctgcaggacgtggtcaaccataatgcccaggcactgaacaccctggtcaagcagctgtcctccaagttcggcgccatctctagcgtgctgaatgacatcttctccaggctggacaaggtggaagccgaggtgcagatcgacagactgatcaccggaaggctgcagtccctgcagacctacgttacccagcagctgattagagccgccgagatcagagccagcgccaatctggctgccacaaagatgagcgagtgtgtgctgggccagagcaagagagtggacttttgcggcaagggctaccacctgatgagcttccctcagtctgctcctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggactccttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccttggtacatctggctgggctttatcgccggactgattgccatcgtgatggtcacaatcatgctgtgctgtatgaccagctgctgtagctgcctgaagggctgttgtagctgtggctcctgctgcaagttcgacgaggacgattctgagcccgtgctgaaaggcgtgaagctgcactacacatga

SEQ ID NO: 16 - protein, 1270 aa, synthetic construct
  REGION 1..1270: note = S-Omicron protein of SARS-CoV-2
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 17 - DNA, 3813 bp, synthetic construct
  misc_feature 1..3813: note = Nucleotide sequence of the polynucleotide encoding S-Omicron-2P protein of SARS-CoV-2
  source 1..3813: mol_type = other DNA
atgttcgtgtttctggtgctgctgcctctggtgtccagccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgtgatcagcggcaccaatggcaccaagagattcgacaaccccgtgctgcccttcaacgacggggtgtactttgccagcatcgagaagtccaacatcatccgcggctggatcttcggcaccacactggatagcaagacccagagcctgctgatcgtgaacaacgccaccaacgtggtcatcaaagtgtgcgagttccagttctgcaacgacccattcctggaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcatcgtgcgcgagcctgaggatctgcctcagggcttttctgccctggaacctctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctcttgatcccctgagcgagacaaagtgcaccctgaagtccttcaccgtggaaaagggcatctaccagaccagcaacttccgggtgcagcccaccgaatccatcgtgcggttccccaatatcaccaatctgtgccccttcgatgaggtgttcaatgccaccagattcgccagcgtgtacgcctggaaccggaagagaatcagcaactgcgtggccgactactccgtgctgtacaatctggccccattcttcaccttcaagtgctacggcgtgtcccctaccaagctgaacgacctgtgcttcaccaatgtgtacgccgacagcttcgtgatccggggagatgaagtgcggcagattgcccctggacagaccggcaatatcgccgactacaactacaagctgcccgacgacttcaccggctgtgtgatcgcctggaatagcaacaagctggacagcaaggtgtccggcaactacaattacctgtaccggctgttccggaagtccaatctgaagcccttcgagcgggacatcagcaccgagatctatcaggccggcaacaagccctgtaatggcgtggccggcttcaactgctacttcccactgcggagctacagcttcagacccacatacggcgttggccaccagccttacagagtggtggtgctgtccttcgagctgctgcatgctcctgccacagtgtgcggccctaagaaaagcaccaacctcgtgaagaacaaatgcgtgaacttcaacttcaacggcctgaaaggcaccggcgtgctgaccgagagcaacaagaagttcctgccattccagcagttcggccgggacattgccgataccacagatgctgtcagagatccccagacactggaaatcctggacatcaccccttgcagcttcggcggagtgtctgtgatcacccctggcaccaacaccagcaatcaggtggcagtgctgtaccagggcgtgaactgtacagaggtgccagtggccattcacgccgatcagctgacccctacttggcgggtgtactccacaggcagcaatgtgttccagaccagagccggctgtctgattggcgccgagtatgtgaacaacagctacgagtgcgacatccccatcggagccggcatctgtgccagctaccagacacagaccaagagccacagacgggctagaagcgtggccagccagagcatcattgcctacacaatgtctctgggcgccgagaacagcgtggcctacagcaacaactctatcgctatccccaccaacttcaccatcagcgtgaccaccgagattctgcccgtgtccatgaccaagaccagcgtggactgcaccatgtacatctgcggcgattccaccgagtgctccaacctgctgctgcagtacggcagcttctgcacccagctgaagagagccctgacagggattgccgtggaacaggacaagaacacccaagaggtgttcgcccaagtgaagcagatctacaagacccctcctatcaagtacttcggcgggttcaacttctcccagatcctgccagatcctagcaagcccagcaagcggagcttcatcgaggacctgctgttcaacaaagtgacactggccgacgccggctttatcaagcagtatggcgattgcctgggcgacattgcagccagggatctgatttgcgcccagaagttcaagggcctgacagtgctgcctcctctgctgacagatgagatgatcgcccagtacacaagcgccctgctggccggcacaatcacctctggatggacatttggagccggcgctgccctgcagatcccatttgctatgcagatggcctaccggttcaacggcatcggagtgacccagaatgtgctgtacgagaaccagaagctgatcgccaaccagttcaacagcgccatcggcaagatccaggacagcctgagcagcacagcctctgctctgggcaagctgcaggacgtggtcaaccataatgcccaggcactgaacaccctggtcaagcagctgtcctccaagttcggcgccatctctagcgtgctgaatgacatcttctccaggctggacccgccggaagccgaggtgcagatcgacagactgatcaccggaaggctgcagtccctgcagacctacgttacccagcagctgattagagccgccgagatcagagccagcgccaatctggctgccacaaagatgagcgagtgtgtgctgggccagagcaagagagtggacttttgcggcaagggctaccacctgatgagcttccctcagtctgctcctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggactccttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccttggtacatctggctgggctttatcgccggactgattgccatcgtgatggtcacaatcatgctgtgctgtatgaccagctgctgtagctgcctgaagggctgttgtagctgtggctcctgctgcaagttcgacgaggacgattctgagcccgtgctgaaaggcgtgaagctgcactacacatga

SEQ ID NO: 18 - protein, 1270 aa, synthetic construct
  REGION 1..1270: note = S-Omicron-2P protein of SARS-CoV-2
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 19 - DNA, 3825 bp, synthetic construct
  misc_feature 1..3825: note = Nucleotide sequence of the polynucleotide encoding S-2P protein of SARS-CoV-2
  source 1..3825: mol_type = other DNA
atgttcgtgtttctggtgctgctgccactggtgtccagtcagtgcgtgaacctgaccacacgaacacagctgccaccagcctacaccaatagcttcacccgcggagtgtactaccccgacaaggtgttccgcagcagcgtgctgcatagcacccaggatctgtttctgcccttcttcagcaacgtgacctggttccacgccatccacgtgtccggcaccaatggcaccaagcgcttcgataatcccgtgctgcccttcaacgatggcgtgtactttgccagcaccgagaagtccaatatcatccgcggctggatcttcggcaccacactggatagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgcgtgtacagcagcgccaacaactgcaccttcgagtacgtgtcccagccattcctgatggacctggaaggcaagcagggcaacttcaagaacctgcgcgagttcgtgttcaagaacatcgacggctacttcaaaatctacagcaagcacaccccaatcaacctcgtgcgcgatctgccacagggattcagtgctctggaacccctggtggatctgcccatcggcatcaacattacccgctttcagacactgctggccctgcaccgcagttacttgacaccaggcgatagcagcagtggatggacagctggtgccgccgcttactacgttggatatctgcagccacgcacctttctgctgaagtacaacgagaacggcaccatcaccgacgccgtggattgtgctctcgatcccctgagcgagacaaagtgcaccctgaagtccttcaccgtcgagaagggcatctaccagaccagcaatttccgcgtgcagcccaccgagagcatcgtgcgcttccccaatatcaccaatctgtgccccttcggcgaggtgttcaatgccacacgctttgcctccgtgtacgcctggaatcgcaagcgcattagcaactgcgtggccgactactccgtgctgtacaatagcgccagcttcagcaccttcaagtgctacggcgtgtcacccaccaagctgaacgacctgtgcttcaccaatgtgtacgccgacagcttcgtgatccgcggagatgaagtgcgacagattgccccaggccagaccggcaagatcgccgactacaattacaagctgcccgacgacttcaccggctgcgtgatcgcctggaacagcaacaacctggattccaaagtcggcggcaactacaactacctgtaccgcctgttccgcaagagcaatctgaagcccttcgagcgcgacatcagcaccgaaatctaccaggccggaagcaccccatgcaacggcgtggaaggcttcaactgctacttcccactgcagtcctacggatttcagcccacaaatggcgtgggctaccagccatatcgagtggtggtgctgagcttcgaactgctgcatgctccagctaccgtgtgcggccccaagaagagtaccaacctggtcaagaacaaatgcgtgaacttcaacttcaacggcctgaccggaaccggcgtgctgaccgagagtaacaagaagttcctgccattccagcagtttggccgcgacattgccgatacaaccgatgccgttcgcgatccccagaccttggagatcctggatattaccccatgctccttcggcggcgtgtccgtgattacaccaggcaccaataccagcaaccaggtggccgttctgtaccaggatgtgaattgcacagaggtgcccgtggccattcacgccgatcaattgacaccaacatggcgcgtgtactccaccggcagcaatgtgtttcaaacccgcgctggatgcctgattggagccgagcacgtgaacaatagctacgagtgcgatatccccatcggagccggaatctgcgcctcctatcagacccagaccaatagtccacgacgagcccgaagtgtggccagccagagcatcattgcctataccatgagcctgggcgccgagaatagcgtggcctactccaacaacagcattgctatccccaccaacttcaccatcagcgtgaccaccgagatcctgccagtgtccatgaccaagaccagcgtggactgcaccatgtacatctgcggagatagcaccgagtgcagcaacctgctgctgcagtacggaagtttctgcacccagctgaatcgcgccctgacaggcattgccgtggaacaggataagaacacccaagaggtgttcgcccaagtgaagcaaatctacaagaccccaccaatcaaggatttcggcggcttcaatttcagccagattctgcccgatccaagcaagcccagcaagcgcagcttcatcgaggacctgctgttcaacaaagtgacactggccgacgccggattcatcaagcagtatggcgattgcctgggcgatattgccgcacgcgatctgatttgcgcccagaagtttaacggactgaccgtcctgccaccactgctgacagatgagatgatcgcccagtacacaagtgccctgctggccggaaccattaccagcggatggacatttggagccggtgccgctctgcagattcccttcgctatgcagatggcctaccgcttcaatggcattggcgtgacccagaatgtgctgtacgagaaccagaagctgatcgccaaccagttcaacagcgccatcggcaagattcaggacagcctgagtagtaccgccagcgctctgggaaagctgcaggatgtggtcaaccagaacgctcaggccctgaacaccctggttaagcagctgagcagcaacttcggcgccatcagtagcgtgctgaacgatatcctgagccgcctggatccaccagaagccgaggtgcagatcgatcgcctgattaccggacgcctgcagtccctgcagacctatgtgacacagcagctgatccgagccgccgagattcgagctagtgctaatctggccgccaccaagatgagcgaatgtgtgctgggacagagcaagcgcgtggacttttgcggcaagggataccacctgatgagcttcccacagagtgctccacacggcgtggtgtttctgcatgtgacctacgtgcccgctcaagagaagaatttcaccaccgctccagccatctgccacgacggaaaggcccattttccacgcgagggcgtgttcgttagcaacggcactcattggttcgtcacccagcgcaacttctacgagccccagatcatcaccaccgacaacaccttcgtcagcggcaactgcgacgtcgtgatcggcattgtgaacaacaccgtgtacgatccactgcagcccgagctggacagcttcaaagaggaactggacaagtactttaagaaccacacaagccccgacgtggacctgggagacattagcggaatcaacgccagcgtggtcaacatccagaaagagattgaccgcctgaacgaggtggccaagaatctgaacgagagcctgatcgacctgcaagaactgggcaaatacgagcagtacattaagtggccctggtacatctggctgggcttcattgccggactgattgccatcgtgatggtcaccattatgctgtgctgcatgaccagttgctgcagctgcctgaagggatgctgcagttgcggaagctgctgcaagttcgacgaggatgatagcgagccagtgctgaagggcgtcaagctgcactacacctgataa

SEQ ID NO: 20 - protein, 1273 aa, synthetic construct
  REGION 1..1273: note = S-2P protein of SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 21 - protein, 4 aa, synthetic construct
  REGION 1..4: note = Peptide
RRAR

SEQ ID NO: 22 - DNA, 10096 bp, synthetic construct
  misc_feature 1..10096: note = Nucleotide sequence of pFlap-ieCMV-S-B351-2P-WPREm
  source 1..10096: mol_type = other DNA
ggcgttgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcccgacattgattattgactagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggctgatacgcgtggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgcgatcggatccacaagtttgtacaaaaaagcaggctgccaccatgttcgtgtttctggtgctgctgccactggtgtccagtcagtgcgtgaacttcaccacacgaacacagctgccaccagcctacaccaatagcttcacccgcggagtgtactaccccgacaaggtgttccgcagcagcgtgctgcatagcacccaggatctgtttctgcccttcttcagcaacgtgacctggttccacgccatccacgtgtccggcaccaatggcaccaagcgcttcgctaatcccgtgctgcccttcaacgatggcgtgtactttgccagcaccgagaagtccaatatcatccgcggctggatcttcggcaccacactggatagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgcgtgtacagcagcgccaacaactgcaccttcgagtacgtgtcccagccattcctgatggacctggaaggcaagcagggcaacttcaagaacctgcgcgagttcgtgttcaagaacatcgacggctacttcaaaatctacagcaagcacaccccaatcaacctcgtgcgcggcctgccacagggattcagtgctctggaacccctggtggatctgcccatcggcatcaacattacccgctttcagacactgcaccgcagttacttgacaccaggcgatagcagcagtggatggacagctggtgccgccgcttactacgttggatatctgcagccacgcacctttctgctgaagtacaacgagaacggcaccatcaccgacgccgtggattgtgctctcgatcccctgagcgagacaaagtgcaccctgaagtccttcaccgtcgagaagggcatctaccagaccagcaatttccgcgtgcagcccaccgagagcatcgtgcgcttccccaatatcaccaatctgtgccccttcggcgaggtgttcaatgccacacgctttgcctccgtgtacgcctggaatcgcaagcgcattagcaactgcgtggccgactactccgtgctgtacaatagcgccagcttcagcaccttcaagtgctacggcgtgtcacccaccaagctgaacgacctgtgcttcaccaatgtgtacgccgacagcttcgtgatccgcggagatgaagtgcgacagattgccccaggccagaccggcaacatcgccgactacaattacaagctgcccgacgacttcaccggctgcgtgatcgcctggaacagcaacaacctggattccaaagtcggcggcaactacaactacctgtaccgcctgttccgcaagagcaatctgaagcccttcgagcgcgacatcagcaccgaaatctaccaggccggaagcaccccatgcaacggcgtgaaaggcttcaactgctacttcccactgcagtcctacggatttcagcccacatatggcgtgggctaccagccatatcgagtggtggtgctgagcttcgaactgctgcatgctccagctaccgtgtgcggccccaagaagagtaccaacctggtcaagaacaaatgcgtgaacttcaacttcaacggcctgaccggaaccggcgtgctgaccgagagtaacaagaagttcctgccattccagcagtttggccgcgacattgccgatacaaccgatgccgttcgcgatccccagaccttggagatcctggatattaccccatgctccttcggcggcgtgtccgtgattacaccaggcaccaataccagcaaccaggtggccgttctgtaccagggcgtgaattgcacagaggtgcccgtggccattcacgccgatcaattgacaccaacatggcgcgtgtactccaccggcagcaatgtgtttcaaacccgcgctggatgcctgattggagccgagcacgtgaacaatagctacgagtgcgatatccccatcggagccggaatctgcgcctcctatcagacccagaccaatagtccacgacgagcccgaagtgtggccagccagagcatcattgcctataccatgagcctgggcgtggagaatagcgtggcctactccaacaacagcattgctatccccaccaacttcaccatcagcgtgaccaccgagatcctgccagtgtccatgaccaagaccagcgtggactgcaccatgtacatctgcggagatagcaccgagtgcagcaacctgctgctgcagtacggaagtttctgcacccagctgaatcgcgccctgacaggcattgccgtggaacaggataagaacacccaagaggtgttcgcccaagtgaagcaaatctacaagaccccaccaatcaaggatttcggcggcttcaatttcagccagattctgcccgatccaagcaagcccagcaagcgcagcttcatcgaggacctgctgttcaacaaagtgacactggccgacgccggattcatcaagcagtatggcgattgcctgggcgatattgccgcacgcgatctgatttgcgcccagaagtttaacggactgaccgtcctgccaccactgctgacagatgagatgatcgcccagtacacaagtgccctgctggccggaaccattaccagcggatggacatttggagccggtgccgctctgcagattcccttcgctatgcagatggcctaccgcttcaatggcattggcgtgacccagaatgtgctgtacgagaaccagaagctgatcgccaaccagttcaacagcgccatcggcaagattcaggacagcctgagtagtaccgccagcgctctgggaaagctgcaggatgtggtcaaccagaacgctcaggccctgaacaccctggttaagcagctgagcagcaacttcggcgccatcagtagcgtgctgaacgatatcctgagccgcctggatccgccggaagccgaggtgcagatcgatcgcctgattaccggacgcctgcagtccctgcagacctatgtgacacagcagctgatccgagccgccgagattcgagctagtgctaatctggccgccaccaagatgagcgaatgtgtgctgggacagagcaagcgcgtggacttttgcggcaagggataccacctgatgagcttcccacagagtgctccacacggcgtggtgtttctgcatgtgacctacgtgcccgctcaagagaagaatttcaccaccgctccagccatctgccacgacggaaaggcccattttccacgcgagggcgtgttcgttagcaacggcactcattggttcgtcacccagcgcaacttctacgagccccagatcatcaccaccgacaacaccttcgtcagcggcaactgcgacgtcgtgatcggcattgtgaacaacaccgtgtacgatccactgcagcccgagctggacagcttcaaagaggaactggacaagtactttaagaaccacacaagccccgacgtggacctgggagacattagcggaatcaacgccagcgtggtcaacatccagaaagagattgaccgcctgaacgaggtggccaagaatctgaacgagagcctgatcgacctgcaagaactgggcaaatacgagcagtacattaagtggccctggtacatctggctgggcttcattgccggactgattgccatcgtgatggtcaccattatgctgtgctgcatgaccagttgctgcagctgcctgaagggatgctgcagttgcggaagctgctgcaagttcgacgaggatgatagcgagccagtgctgaagggcgtcaagctgcactacacctgatagtaaacccagctttcttgtacaaagtggtctcgagttcccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcggtacctttaagaccaatgacttacaaggcagctgtagatcttagccactttttaaaagaaaaggggggactggaagggctaattcactcccaacgaagacaaaatcgtcgagagatgctgcatataagcagctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagttctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcttgctgcttcgcgatgtacgggccagatatacgc

SEQ ID NO: 23 - DNA, 3822 bp, synthetic construct
  source 1..3822: mol_type = other DNA
  misc_feature 1..3822: note = Nucleotide sequence of the polynucleotide encoding S-Omicron-BA.1 protein of SARS-CoV-2
atgttcgtgttcctggtgctgctgcctctggtgagcagccagtgcgtgaacctgaccaccaggacccagctgcctcctgcctacaccaacagcttcaccaggggcgtgtactaccctgacaaggtgttcaggagcagcgtgctgcacagcacccaggacctgttcctgcctttcttcagcaacgtgacctggttccacgccatccacgtgagcggcaccaacggcaccaagaggttcgacaaccctgtgctgcctttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccctttcctgggcgtgtactaccacaagaacaacaagagctggatggagagcgagttcagggtgtacagcagcgccaacaactgcaccttcgagtacgtgagccagcctttcctgatggacctggagggcaagcagggcaacttcaagaacctgagggagttcgtgttcaagaacatcgacggctacttcaagatctacagcaagcacacccctatcaacctggtgagggacctgcctcagggcttcagcgccctggagcctctggtggacctgcctatcggcatcaacatcaccaggttccagaccctgctggccctgcacaggagctacctgacccctggcgacagcagcagcggctggaccgccggcgccgccgcctactacgtgggctacctgcagcctaggaccttcctgctgaagtacaacgagaacggcaccatcaccgacgccgtggactgcgccctggaccctctgagcgagaccaagtgcaccctgaagagcttcaccgtggagaagggcatctaccagaccagcaacttcagggtgcagcctaccgagagcatcgtgaggttccctaacatcaccaacctgtgccctttcggcgaggtgttcaacgccaccaggttcgccagcgtgtacgcctggaacaggaagaggatcagcaactgcgtggccgactacagcgtgctgtacaacagcgccagcttcagcaccttcaagtgctacggcgtgagccctaccaagctgaacgacctgtgcttcaccaacgtgtacgccgacagcttcgtgatcaggggcgacgaggtgaggcagatcgcccctggccagaccggcaagatcgccgactacaactacaagctgcctgacgacttcaccggctgcgtgatcgcctggaacagcaacaacctggacagcaaggtgggcggcaactacaactacctgtacaggctgttcaggaagagcaacctgaagcctttcgagagggacatcagcaccgagatctaccaggccggcagcaccccttgcaacggcgtggagggcttcaactgctacttccctctgcagagctacggcttccagcctaccaacggcgtgggctaccagccttacagggtggtggtgctgagcttcgagctgctgcacgcccctgccaccgtgtgcggccctaagaagagcaccaacctggtgaagaacaagtgcgtgaacttcaacttcaacggcctgaccggcaccggcgtgctgaccgagagcaacaagaagttcctgcctttccagcagttcggcagggacatcgccgacaccaccgacgccgtgagggaccctcagaccctggagatcctggacatcaccccttgcagcttcggcggcgtgagcgtgatcacccctggcaccaacaccagcaaccaggtggccgtgctgtaccagggcgtgaactgcaccgaggtgcctgtggccatccacgccgaccagctgacccctacctggagggtgtacagcaccggcagcaacgtgttccagaccagggccggctgcctgatcggcgccgagcacgtgaacaacagctacgagtgcgacatccctatcggcgccggcatctgcgccagctaccagacccagaccaacagccctaggagggccaggagcgtggccagccagagcatcatcgcctacaccatgagcctgggcgccgagaacagcgtggcctacagcaacaacagcatcgccatccctaccaacttcaccatcagcgtgaccaccgagatcctgcctgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagacccctcctatcaaggacttcggcggcttcaacttcagccagatcctgcctgaccctagcaagcctagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgcctcctctgctgaccgacgagatgatcgcccagtacaccagcgccctgctggccggcaccatcaccagcggctggaccttcggcgccggcgccgccctgcagatccctttcgccatgcagatggcctacaggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgacatcctgagcaggctggacaaggtggaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttccctcagagcgcccctcacggcgtggtgttcctgcacgtgacctacgtgcctgcccaggagaagaacttcaccaccgcccctgccatctgccacgacggcaaggcccacttccctagggagggcgtgttcgtgagcaacggcacccactggttcgtgacccagaggaacttctacgagcctcagatcatcaccaccgacaacaccttcgtgagcggcaactgcgacgtggtgatcggcatcgtgaacaacaccgtgtacgaccctctgcagcctgagctggacagcttcaaggaggagctggacaagtacttcaagaaccacaccagccctgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccttggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcctgtgctgaagggcgtgaagctgcactacacctga

SEQ ID NO: 24 - protein, 1273 aa, synthetic construct
  REGION 1..1273: note = S-Omicron-BA.1 protein of SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 25 - DNA, 3807 bp, synthetic construct
  source 1..3807: mol_type = other DNA
  misc_feature 1..3807: note = Nucleotide sequence of the polynucleotide encoding S-Omicron-BA.4/5 protein of SARS-CoV-2
atgttcgtgtttctggtgctgctgccactggtgtccagtcagtgcgtgaacctgatcacacgaacacagagctacaccaatagcttcacccgcggagtgtactaccccgacaaggtgttccgcagcagcgtgctgcatagcacccaggatctgtttctgcccttcttcagcaacgtgacctggttccacgccatctccggcaccaatggcaccaagcgcttcgataatcccgtgctgcccttcaacgatggcgtgtactttgccagcaccgagaagtccaatatcatccgcggctggatcttcggcaccacactggatagcaagacccagagcctgctgatcgtgaacaacgccaccaacgtggtcatcaaagtgtgcgagttccagttctgcaacgaccccttcctggacgtctactaccacaagaacaacaagagctggatggaaagcgagttccgcgtgtacagcagcgccaacaactgcaccttcgagtacgtgtcccagccattcctgatggacctggaaggcaagcagggcaacttcaagaacctgcgcgagttcgtgttcaagaacatcgacggctacttcaaaatctacagcaagcacaccccaatcaacctcggccgcgatctgccacagggattcagtgctctggaacccctggtggatctgcccatcggcatcaacattacccgctttcagacactgctggccctgcaccgcagttacttgacaccaggcgatagcagcagtggatggacagctggtgccgccgcttactacgttggatatctgcagccacgcacctttctgctgaagtacaacgagaacggcaccatcaccgacgccgtggattgtgctctcgatcccctgagcgagacaaagtgcaccctgaagtccttcaccgtcgagaagggcatctaccagaccagcaatttccgcgtgcagcccaccgagagcatcgtgcgcttccccaatatcaccaatctgtgccccttcgacgaggtgttcaatgccacacgctttgcctccgtgtacgcctggaatcgcaagcgcattagcaactgcgtggccgactactccgtgctgtacaatttcgcccccttcttcgccttcaagtgctacggcgtgtcacccaccaagctgaacgacctgtgcttcaccaatgtgtacgccgacagcttcgtgatccgcggaaacgaagtgagccagattgccccaggccagaccggcaacatcgccgactacaattacaagctgcccgacgacttcaccggctgcgtgatcgcctggaacagcaacaagctggattccaaagtcggcggcaactacaactaccgttaccgcctgttccgcaagagcaatctgaagcccttcgagcgcgacatcagcaccgaaatctaccaggccggaaacaagccatgcaacggcgtggccggcgtgaactgctacttcccactgcagtcctacggatttcggcccacatacggcgtgggccaccagccatatcgagtggtggtgctgagcttcgaactgctgcatgctccagctaccgtgtgcggccccaagaagagtaccaacctggtcaagaacaaatgcgtgaacttcaacttcaacggcctgaccggaaccggcgtgctgaccgagagtaacaagaagttcctgccattccagcagtttggccgcgacattgccgatacaaccgatgccgttcgcgatccccagaccttggagatcctggatattaccccatgctccttcggcggcgtgtccgtgattacaccaggcaccaataccagcaaccaggtggccgttctgtaccagggcgtgaattgcacagaggtgcccgtggccattcacgccgatcaattgacaccaacatggcgcgtgtactccaccggcagcaatgtgtttcaaacccgcgctggatgcctgattggagccgagtacgtgaacaatagctacgagtgcgatatccccatcggagccggaatctgcgcctcctatcagacccagaccaagagtcaccgacgagcccgaagtgtggccagccagagcatcattgcctataccatgagcctgggcgccgagaatagcgtggcctactccaacaacagcattgctatccccaccaacttcaccatcagcgtgaccaccgagatcctgccagtgtccatgaccaagaccagcgtggactgcaccatgtacatctgcggagatagcaccgagtgcagcaacctgctgctgcagtacggaagtttctgcacccagctgaagcgcgccctgacaggcattgccgtggaacaggataagaacacccaagaggtgttcgcccaagtgaagcaaatctacaagaccccaccaatcaagtacttcggcggcttcaatttcagccagattctgcccgatccaagcaagcccagcaagcgcagcttcatcgaggacctgctgttcaacaaagtgacactggccgacgccggattcatcaagcagtatggcgattgcctgggcgatattgccgcacgcgatctgatttgcgcccagaagtttaacggactgaccgtcctgccaccactgctgacagatgagatgatcgcccagtacacaagtgccctgctggccggaaccattaccagcggatggacatttggagccggtgccgctctgcagattcccttcgctatgcagatggcctaccgcttcaatggcattggcgtgacccagaatgtgctgtacgagaaccagaagctgatcgccaaccagttcaacagcgccatcggcaagattcaggacagcctgagtagtaccgccagcgctctgggaaagctgcaggatgtggtcaaccacaacgctcaggccctgaacaccctggttaagcagctgagcagcaagttcggcgccatcagtagcgtgctgaacgatatcctgagccgcctggataaggtggaagccgaggtgcagatcgatcgcctgattaccggacgcctgcagtccctgcagacctatgtgacacagcagctgatccgagccgccgagattcgagctagtgctaatctggccgccaccaagatgagcgaatgtgtgctgggacagagcaagcgcgtggacttttgcggcaagggataccacctgatgagcttcccacagagtgctccacacggcgtggtgtttctgcatgtgacctacgtgcccgctcaagagaagaatttcaccaccgctccagccatctgccacgacggaaaggcccattttccacgcgagggcgtgttcgttagcaacggcactcattggttcgtcacccagcgcaacttctacgagccccagatcatcaccaccgacaacaccttcgtcagcggcaactgcgacgtcgtgatcggcattgtgaacaacaccgtgtacgatccactgcagcccgagctggacagcttcaaagaggaactggacaagtactttaagaaccacacaagccccgacgtggacctgggagacattagcggaatcaacgccagcgtggtcaacatccagaaagagattgaccgcctgaacgaggtggccaagaatctgaacgagagcctgatcgacctgcaagaactgggcaaatacgagcagtacattaagtggccctggtacatctggctgggcttcattgccggactgattgccatcgtgatggtcaccattatgctgtgctgcatgaccagttgctgcagctgcctgaagggatgctgcagttgcggaagctgctgcaagttcgacgaggatgatagcgagccagtgctgaagggcgtcaagctgcactacacctga

SEQ ID NO: 26 - protein, 1268 aa, synthetic construct
  REGION 1..1268: note = S-Omicron-BA.4/5 protein of SARS-CoV-2
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 27 - protein, 11 aa, synthetic construct
  REGION 1..11: note = loop encompassing the S1/S2 furin cleavage site of the S protein of SARS-CoV-2
QTQTNSPRRAR